SEQUENCE LISTING (54 sequences)

SEQ ID NO: 1 - DNA, 19 bp, Homo sapiens
gaaggtgaaggtcggagtc

SEQ ID NO: 2 - DNA, 20 bp, Homo sapiens
gaagatggtgatgggatttc

SEQ ID NO: 3 - DNA, 20 bp, Homo sapiens
tgccccgtcttatggaaacc

SEQ ID NO: 4 - DNA, 20 bp, Homo sapiens
ctcttcttctgggcccactc

SEQ ID NO: 5 - DNA, 20 bp, Homo sapiens
actgcccacacgataaggag

SEQ ID NO: 6 - DNA, 20 bp, Homo sapiens
ctggctatgtctttgcacca

SEQ ID NO: 7 - DNA, 20 bp, Homo sapiens
tgcagctatacccaggctgg

SEQ ID NO: 8 - DNA, 19 bp, Homo sapiens
cctcgaccgcctcttcttc

SEQ ID NO: 9 - DNA, 20 bp, Homo sapiens
taccctctcaacgacagcag

SEQ ID NO: 10 - DNA, 20 bp, Homo sapiens
tcttgacattctcctcggtg

SEQ ID NO: 11 - DNA, 20 bp, Homo sapiens
cggcgtttatcttcgctatc

SEQ ID NO: 12 - DNA, 20 bp, Homo sapiens
ttcgatggaaccttctgagc

SEQ ID NO: 13 - DNA, 20 bp, Homo sapiens
aaagaagatgggaagcgcca

SEQ ID NO: 14 - DNA, 20 bp, Homo sapiens
ttcatcagggctctggcatc

SEQ ID NO: 15 - DNA, 20 bp, Homo sapiens
tgttgggccacagtattcct

SEQ ID NO: 16 - DNA, 20 bp, Homo sapiens
gggcatgatctccacgtagt

SEQ ID NO: 17 - DNA, 19 bp, Homo sapiens
cagccccttggttatggac

SEQ ID NO: 18 - DNA, 20 bp, Homo sapiens
aactggaactggcactctcg

SEQ ID NO: 19 - DNA, 21 bp, Homo sapiens
aggctgagaagtgctaccaga

SEQ ID NO: 20 - DNA, 20 bp, Homo sapiens
ccattgtttgacccatcaca

SEQ ID NO: 21 - DNA, 20 bp, Homo sapiens
catcgtcatcgcctgctact

SEQ ID NO: 22 - DNA, 20 bp, Homo sapiens
tagcgtagctcttgcagctc

SEQ ID NO: 23 - DNA, 20 bp, Homo sapiens
cttctcacaggaggagacgc

SEQ ID NO: 24 - DNA, 20 bp, Homo sapiens
aaatgataggccgctctggg

SEQ ID NO: 25 - DNA, 21 bp, Homo sapiens
acagcaaagtgagcagctacc

SEQ ID NO: 26 - DNA, 20 bp, Homo sapiens
ctgtaactgcagggcgtgta

SEQ ID NO: 27 - DNA, 20 bp, Homo sapiens
tcaagagacgctgtgaaccc

SEQ ID NO: 28 - DNA, 20 bp, Homo sapiens
ggtcgttctgtggtgggaat

SEQ ID NO: 29 - DNA, 20 bp, Homo sapiens
gcacaaccacatccactacg

SEQ ID NO: 30 - DNA, 20 bp, Homo sapiens
gcacaaccacatccactacg

SEQ ID NO: 31 - DNA, 20 bp, Homo sapiens
aggcttgcaccgttttgttc

SEQ ID NO: 32 - DNA, 20 bp, Homo sapiens
tgctcgatggcttcacaact

SEQ ID NO: 33 - DNA, 20 bp, Homo sapiens
cgcctctgttcgtctacctc

SEQ ID NO: 34 - DNA, 20 bp, Homo sapiens
tcatgatggtgcggatacgg

SEQ ID NO: 35 - DNA, 20 bp, Homo sapiens
cgcctctgttcgtctacctc

SEQ ID NO: 36 - DNA, 20 bp, Homo sapiens
tcatgatggtgcggatacgg

SEQ ID NO: 37 - DNA, 20 bp, Homo sapiens
tgctcaccttcttgctggag

SEQ ID NO: 38 - DNA, 20 bp, Homo sapiens
gccagcgagtagacgttgta

SEQ ID NO: 39 - DNA, 20 bp, Homo sapiens
aagaagagccggagaaaacc

SEQ ID NO: 40 - DNA, 20 bp, Homo sapiens
gactgggcagggatctcata

SEQ ID NO: 41 - DNA, 20 bp, Homo sapiens
caccaccttcttgctgttca

SEQ ID NO: 42 - DNA, 20 bp, Homo sapiens
gctttgacgttcctcagtcc

SEQ ID NO: 43 - DNA, 20 bp, Homo sapiens
gactgggttgcacgaaatct

SEQ ID NO: 44 - DNA, 20 bp, Homo sapiens
cggggttcctctaagtcctc

SEQ ID NO: 45 - DNA, 20 bp, Homo sapiens
cccggaataactcgagtagg

SEQ ID NO: 46 - DNA, 20 bp, Homo sapiens
gtcactgtaagtgatgaggg

SEQ ID NO: 47 - DNA, 3282 bp, Homo sapiens
atggagtttgggctgagctggctttttcttgtggctattttaaaaggtgtccagtgctctagagatattttgctgacccaaactccactctccctgcctgtcagtcttggagatcaagcctccatctcttgcagatctagtcagagtcttgtacaccgtaatggaaacacctatttacattggtacctgcagaagccaggccagtctccaaagctcctgattcacaaagtttccaaccgattttctggggtcccagacaggttcagtggcagtggatcagggacagatttcacactcaagatcagcagagtggaggctgaggatctgggagtttatttctgttctcaaagtacacatgttcctccgctcacgttcggtgctgggaccaagctggagctgaaacgggctgatgctgcaccaactgtatccatcttcccaggctcgggcggtggtgggtcgggtggcgaggtgaagcttcagcagtctggacctagcctggtggagcctggcgcttcagtgatgatatcctgcaaggcttctggttcctcattcactggctacaacatgaactgggtgaggcagaacattggaaagagccttgaatggattggagctattgatccttactatggtggaactagctacaaccagaagttcaagggcagggccacattgactgtagacaaatcgtccagcacagcctacatgcacctcaagagcctgacatctgaggactctgcagtctattactgtgtaagcggaatggagtactggggtcaaggaacctcagtcaccgtctcctcaacgcgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgtactaattatgctctcttgaaattggctggagatgttgagagcaatcccgggcccatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctgctactaacttcagcctgctgaagcaggctggagatgtggaggagaaccctggacctatgccccaactctccggaggaggtggcggcggcgggggggacccggaactctgcgccacggacgagatgatccccttcaaggacgagggcgatcctcagaaggaaaagatcttcgccgagatcagtcatcccgaagaggaaggcgatttagctgacatcaagtcttccttggtgaacgagtctgaaatcatcccggccagcaacggacacgaggtggccagacaagcacaaacctctcaggagccctaccacgacaaggccagagaacaccccgatgacggaaagcatccagatggaggcctctacaacaagggaccctcctactcgagttattccgggtacataatgatgccaaatatgaataacgacccatacatgtcaaatggatctctttctccacccatcccgagaacatcaaataaagtgcccgtggtgcagccatcccatgcggtccatcctctcacccccctcatcacttacagtgacgagcacttttctccaggatcacacccgtcacacatcccatcagatgtcaactccaaacaaggcatgtccagacatcctccagctcctgatatccctactttttatcccttgtctccgggtggtgttggacagatcaccccacctcttggctggcaaggtcagcctgtatatcccatcacgggtggattcaggcaaccctacccatcctcactgtcagtcgacacttccatgtccaggttttcccatcatatgattcccggtcctcctggtccccacacaactggcatccctcatccagctattgtaacacctcaggtcaaacaggaacatccccacactgacagtgacctaatgcacgtgaagcctcagcatgaacagagaaaggagcaggagccaaaaagacctcacattaagaagcctctgaatgcttttatgttatacatgaaagaaatgagagcgaatgtcgttgctgagtgtactctaaaagaaagtgcagctatcaaccagattcttggcagaaggtggcatgccctctcccgtgaagagcaggctaaatattatgaattagcacggaaagaaagacagctacatatgcagctttatccaggctggtctgcaagagacaattatggtaagaaaaagaagaggaagagagagaaactacaggaatctgcatcaggtacaggtccaagaatgacagctgcctacatctga

SEQ ID NO: 48 - DNA, 2028 bp, Homo sapiens
atggagtttgggctgagctggctttttcttgtggctattttaaaaggtgtccagtgctctagagatattttgctgacccaaactccactctccctgcctgtcagtcttggagatcaagcctccatctcttgcagatctagtcagagtcttgtacaccgtaatggaaacacctatttacattggtacctgcagaagccaggccagtctccaaagctcctgattcacaaagtttccaaccgattttctggggtcccagacaggttcagtggcagtggatcagggacagatttcacactcaagatcagcagagtggaggctgaggatctgggagtttatttctgttctcaaagtacacatgttcctccgctcacgttcggtgctgggaccaagctggagctgaaacgggctgatgctgcaccaactgtatccatcttcccaggctcgggcggtggtgggtcgggtggcgaggtgaagcttcagcagtctggacctagcctggtggagcctggcgcttcagtgatgatatcctgcaaggcttctggttcctcattcactggctacaacatgaactgggtgaggcagaacattggaaagagccttgaatggattggagctattgatccttactatggtggaactagctacaaccagaagttcaagggcagggccacattgactgtagacaaatcgtccagcacagcctacatgcacctcaagagcctgacatctgaggactctgcagtctattactgtgtaagcggaatggagtactggggtcaaggaacctcagtcaccgtctcctcaacgcgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgtactaattatgctctcttgaaattggctggagatgttgagagcaatcccgggcccatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 49 - DNA, 2736 bp, Homo sapiens
atggagtttgggctgagctggctttttcttgtggctattttaaaaggtgtccagtgctctagagatattttgctgacccaaactccactctccctgcctgtcagtcttggagatcaagcctccatctcttgcagatctagtcagagtcttgtacaccgtaatggaaacacctatttacattggtacctgcagaagccaggccagtctccaaagctcctgattcacaaagtttccaaccgattttctggggtcccagacaggttcagtggcagtggatcagggacagatttcacactcaagatcagcagagtggaggctgaggatctgggagtttatttctgttctcaaagtacacatgttcctccgctcacgttcggtgctgggaccaagctggagctgaaacgggctgatgctgcaccaactgtatccatcttcccaggctcgggcggtggtgggtcgggtggcgaggtgaagcttcagcagtctggacctagcctggtggagcctggcgcttcagtgatgatatcctgcaaggcttctggttcctcattcactggctacaacatgaactgggtgaggcagaacattggaaagagccttgaatggattggagctattgatccttactatggtggaactagctacaaccagaagttcaagggcagggccacattgactgtagacaaatcgtccagcacagcctacatgcacctcaagagcctgacatctgaggactctgcagtctattactgtgtaagcggaatggagtactggggtcaaggaacctcagtcaccgtctcctcaacgcgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgctactaacttcagcctgctgaagcaggctggagatgtggaggagaaccctggacctatgccccaactctccggaggaggtggcggcggcgggggggacccggaactctgcgccacggacgagatgatccccttcaaggacgagggcgatcctcagaaggaaaagatcttcgccgagatcagtcatcccgaagaggaaggcgatttagctgacatcaagtcttccttggtgaacgagtctgaaatcatcccggccagcaacggacacgaggtggccagacaagcacaaacctctcaggagccctaccacgacaaggccagagaacaccccgatgacggaaagcatccagatggaggcctctacaacaagggaccctcctactcgagttattccgggtacataatgatgccaaatatgaataacgacccatacatgtcaaatggatctctttctccacccatcccgagaacatcaaataaagtgcccgtggtgcagccatcccatgcggtccatcctctcacccccctcatcacttacagtgacgagcacttttctccaggatcacacccgtcacacatcccatcagatgtcaactccaaacaaggcatgtccagacatcctccagctcctgatatccctactttttatcccttgtctccgggtggtgttggacagatcaccccacctcttggctggcaaggtcagcctgtatatcccatcacgggtggattcaggcaaccctacccatcctcactgtcagtcgacacttccatgtccaggttttcccatcatatgattcccggtcctcctggtccccacacaactggcatccctcatccagctattgtaacacctcaggtcaaacaggaacatccccacactgacagtgacctaatgcacgtgaagcctcagcatgaacagagaaaggagcaggagccaaaaagacctcacattaagaagcctctgaatgcttttatgttatacatgaaagaaatgagagcgaatgtcgttgctgagtgtactctaaaagaaagtgcagctatcaaccagattcttggcagaaggtggcatgccctctcccgtgaagagcaggctaaatattatgaattagcacggaaagaaagacagctacatatgcagctttatccaggctggtctgcaagagacaattatggtaagaaaaagaagaggaagagagagaaactacaggaatctgcatcaggtacaggtccaagaatgacagctgcctacatctga

SEQ ID NO: 50 - DNA, 1482 bp, Homo sapiens
atggagtttgggctgagctggctttttcttgtggctattttaaaaggtgtccagtgctctagagatattttgctgacccaaactccactctccctgcctgtcagtcttggagatcaagcctccatctcttgcagatctagtcagagtcttgtacaccgtaatggaaacacctatttacattggtacctgcagaagccaggccagtctccaaagctcctgattcacaaagtttccaaccgattttctggggtcccagacaggttcagtggcagtggatcagggacagatttcacactcaagatcagcagagtggaggctgaggatctgggagtttatttctgttctcaaagtacacatgttcctccgctcacgttcggtgctgggaccaagctggagctgaaacgggctgatgctgcaccaactgtatccatcttcccaggctcgggcggtggtgggtcgggtggcgaggtgaagcttcagcagtctggacctagcctggtggagcctggcgcttcagtgatgatatcctgcaaggcttctggttcctcattcactggctacaacatgaactgggtgaggcagaacattggaaagagccttgaatggattggagctattgatccttactatggtggaactagctacaaccagaagttcaagggcagggccacattgactgtagacaaatcgtccagcacagcctacatgcacctcaagagcctgacatctgaggactctgcagtctattactgtgtaagcggaatggagtactggggtcaaggaacctcagtcaccgtctcctcaacgcgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 51 - DNA, 3276 bp, Homo sapiens
atggagtttgggctgagctggctttttcttgtggctattttaaaaggtgtccagtgctctagagatattttgctgacccaaactccactctccctgcctgtcagtcttggagatcaagcctccatctcttgcagatctagtcagagtcttgtacaccgtaatggaaacacctatttacattggtacctgcagaagccaggccagtctccaaagctcctgattcacaaagtttccaaccgattttctggggtcccagacaggttcagtggcagtggatcagggacagatttcacactcaagatcagcagagtggaggctgaggatctgggagtttatttctgttctcaaagtacacatgttcctccgctcacgttcggtgctgggaccaagctggagctgaaacgggctgatgctgcaccaactgtatccatcttcccaggctcgggcggtggtgggtcgggtggcgaggtgaagcttcagcagtctggacctagcctggtggagcctggcgcttcagtgatgatatcctgcaaggcttctggttcctcattcactggctacaacatgaactgggtgaggcagaacattggaaagagccttgaatggattggagctattgatccttactatggtggaactagctacaaccagaagttcaagggcagggccacattgactgtagacaaatcgtccagcacagcctacatgcacctcaagagcctgacatctgaggactctgcagtctattactgtgtaagcggaatggagtactggggtcaaggaacctcagtcaccgtctcctcaacgcgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgtactaattatgctctcttgaaattggctggagatgttgagagcaatcccgggcccatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttctgctactaacttcagcctgctgaagcaggctggagatgtggaggagaaccctggacctatgccccaactctccggaggaggtggcggcggcgggggggacccggaactctgcgccacggacgagatgatccccttcaaggacgagggcgatcctcagaaggaaaagatcttcgccgagatcagtcatcccgaagaggaaggcgatttagctgacatcaagtcttccttggtgaacgagtctgaaatcatcccggccagcaacggacacgaggtggccagacaagcacaaacctctcaggagccctaccacgacaaggccagagaacaccccgatgacggaaagcatccagatggaggcctctacaacaagggaccctcctactcgagttattccgggtacataatgatgccaaatatgaataacgacccatacatgtcaaatggatctctttctccacccatcccgagaacatcaaataaagtgcccgtggtgcagccatcccatgcggtccatcctctcacccccctcatcacttacagtgacgagcacttttctccaggatcacacccgtcacacatcccatcagatgtcaactccaaacaaggcatgtccagacatcctccagctcctgatatccctactttttatcccttgtctccgggtggtgttggacagatcaccccacctcttggctggcaaggtcagcctgtatatcccatcacgggtggattcaggcaaccctacccatcctcactgtcagtcgacacttccatgtccaggttttcccatcatatgattcccggtcctcctggtccccacacaactggcatccctcatccagctattgtaacacctcaggtcaaacaggaacatccccacactgacagtgacctaatgcacgtgaagcctcagcatgaacagagaaaggagcaggagccaaaaagacctcacattaagaagcctctgaatgcttttatgttatacatgaaagaaatgagagcgaatgtcgttgctgagtgtactctaaaagaaagtgcagctatcaaccagattcttggcagaaggtggcatgccctctcccgtgaagagcaggctaaatattatgaattagcacggaaagaaagacagctacatatgcagctttatccaggctggtctgcaagagacaattatggtaagaaaaagaagaggaagagagagaaactacaggaatctgcatcaggtacaggtccaagaatgacagctgcctacatc

SEQ ID NO: 52 - DNA, 2025 bp, Homo sapiens
atggagtttgggctgagctggctttttcttgtggctattttaaaaggtgtccagtgctctagagatattttgctgacccaaactccactctccctgcctgtcagtcttggagatcaagcctccatctcttgcagatctagtcagagtcttgtacaccgtaatggaaacacctatttacattggtacctgcagaagccaggccagtctccaaagctcctgattcacaaagtttccaaccgattttctggggtcccagacaggttcagtggcagtggatcagggacagatttcacactcaagatcagcagagtggaggctgaggatctgggagtttatttctgttctcaaagtacacatgttcctccgctcacgttcggtgctgggaccaagctggagctgaaacgggctgatgctgcaccaactgtatccatcttcccaggctcgggcggtggtgggtcgggtggcgaggtgaagcttcagcagtctggacctagcctggtggagcctggcgcttcagtgatgatatcctgcaaggcttctggttcctcattcactggctacaacatgaactgggtgaggcagaacattggaaagagccttgaatggattggagctattgatccttactatggtggaactagctacaaccagaagttcaagggcagggccacattgactgtagacaaatcgtccagcacagcctacatgcacctcaagagcctgacatctgaggactctgcagtctattactgtgtaagcggaatggagtactggggtcaaggaacctcagtcaccgtctcctcaacgcgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgtactaattatgctctcttgaaattggctggagatgttgagagcaatcccgggcccatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 53 - DNA, 2730 bp, Homo sapiens
atggagtttgggctgagctggctttttcttgtggctattttaaaaggtgtccagtgctctagagatattttgctgacccaaactccactctccctgcctgtcagtcttggagatcaagcctccatctcttgcagatctagtcagagtcttgtacaccgtaatggaaacacctatttacattggtacctgcagaagccaggccagtctccaaagctcctgattcacaaagtttccaaccgattttctggggtcccagacaggttcagtggcagtggatcagggacagatttcacactcaagatcagcagagtggaggctgaggatctgggagtttatttctgttctcaaagtacacatgttcctccgctcacgttcggtgctgggaccaagctggagctgaaacgggctgatgctgcaccaactgtatccatcttcccaggctcgggcggtggtgggtcgggtggcgaggtgaagcttcagcagtctggacctagcctggtggagcctggcgcttcagtgatgatatcctgcaaggcttctggttcctcattcactggctacaacatgaactgggtgaggcagaacattggaaagagccttgaatggattggagctattgatccttactatggtggaactagctacaaccagaagttcaagggcagggccacattgactgtagacaaatcgtccagcacagcctacatgcacctcaagagcctgacatctgaggactctgcagtctattactgtgtaagcggaatggagtactggggtcaaggaacctcagtcaccgtctcctcaacgcgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgctactaacttcagcctgctgaagcaggctggagatgtggaggagaaccctggacctatgccccaactctccggaggaggtggcggcggcgggggggacccggaactctgcgccacggacgagatgatccccttcaaggacgagggcgatcctcagaaggaaaagatcttcgccgagatcagtcatcccgaagaggaaggcgatttagctgacatcaagtcttccttggtgaacgagtctgaaatcatcccggccagcaacggacacgaggtggccagacaagcacaaacctctcaggagccctaccacgacaaggccagagaacaccccgatgacggaaagcatccagatggaggcctctacaacaagggaccctcctactcgagttattccgggtacataatgatgccaaatatgaataacgacccatacatgtcaaatggatctctttctccacccatcccgagaacatcaaataaagtgcccgtggtgcagccatcccatgcggtccatcctctcacccccctcatcacttacagtgacgagcacttttctccaggatcacacccgtcacacatcccatcagatgtcaactccaaacaaggcatgtccagacatcctccagctcctgatatccctactttttatcccttgtctccgggtggtgttggacagatcaccccacctcttggctggcaaggtcagcctgtatatcccatcacgggtggattcaggcaaccctacccatcctcactgtcagtcgacacttccatgtccaggttttcccatcatatgattcccggtcctcctggtccccacacaactggcatccctcatccagctattgtaacacctcaggtcaaacaggaacatccccacactgacagtgacctaatgcacgtgaagcctcagcatgaacagagaaaggagcaggagccaaaaagacctcacattaagaagcctctgaatgcttttatgttatacatgaaagaaatgagagcgaatgtcgttgctgagtgtactctaaaagaaagtgcagctatcaaccagattcttggcagaaggtggcatgccctctcccgtgaagagcaggctaaatattatgaattagcacggaaagaaagacagctacatatgcagctttatccaggctggtctgcaagagacaattatggtaagaaaaagaagaggaagagagagaaactacaggaatctgcatcaggtacaggtccaagaatgacagctgcctacatc

SEQ ID NO: 54 - DNA, 1479 bp, Homo sapiens
atggagtttgggctgagctggctttttcttgtggctattttaaaaggtgtccagtgctctagagatattttgctgacccaaactccactctccctgcctgtcagtcttggagatcaagcctccatctcttgcagatctagtcagagtcttgtacaccgtaatggaaacacctatttacattggtacctgcagaagccaggccagtctccaaagctcctgattcacaaagtttccaaccgattttctggggtcccagacaggttcagtggcagtggatcagggacagatttcacactcaagatcagcagagtggaggctgaggatctgggagtttatttctgttctcaaagtacacatgttcctccgctcacgttcggtgctgggaccaagctggagctgaaacgggctgatgctgcaccaactgtatccatcttcccaggctcgggcggtggtgggtcgggtggcgaggtgaagcttcagcagtctggacctagcctggtggagcctggcgcttcagtgatgatatcctgcaaggcttctggttcctcattcactggctacaacatgaactgggtgaggcagaacattggaaagagccttgaatggattggagctattgatccttactatggtggaactagctacaaccagaagttcaagggcagggccacattgactgtagacaaatcgtccagcacagcctacatgcacctcaagagcctgacatctgaggactctgcagtctattactgtgtaagcggaatggagtactggggtcaaggaacctcagtcaccgtctcctcaacgcgtaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

(19) United States
(12) Patent Application Publication
(10) Pub. No.: US 2024/0110154 A1
(43) Pub. Date: Apr. 4, 2024

(54) Engineered NKT Cells for Expansion and In Vivo Preservation and Methods of Use for the Control of Tumor Cells

(71) Applicant: Baylor College of Medicine, Houston, TX (US)

(72) Inventors: Leonid S. METELITSA, Sugar Land, TX (US); Ho NGAI, Houston, TX (US)

(73) Assignee: Baylor College of Medicine, Houston, TX (US)

(21) Appl. No.: 18/276,164

(22) PCT Filed: Feb. 7, 2022
(86) PCT No.: PCT/US2022/015525

§ 371 Date: Aug. 7, 2023

Related U.S. Application Data
(60) Provisional application No. 63/146,693, filed on Feb. 7, 2021.

(51) Int. Cl. C12N 5/0783; C07K 14/705; C07K 14/725; C07K 16/28; C07K 16/30

(52) U.S. Cl. CPC C12N 5/0646; C07K 14/7051; C07K 14/70578; C07K 16/2803; C07K 16/3084; C07K 2317/622; C12N 2501/415; C12N 2510/00

(57) ABSTRACT

The present disclosure relates to methods and compositions related to Natural Killer T cells that are engineered to harbor an expression construct that encodes an activator of the Wnt signaling pathway. Activation by expression of Wnt signaling activators or the addition of exogenous activators promotes NKT expansion over the course of multiple tumor cell challenges and improves long term tumor control in vitro. The present disclosure further includes NKT cells, populations, and methods to prepare them, that are engineered to express exogenous activators of Wnt signaling combined with chimeric antigen receptors (CARs) for therapeutic use.

### CROSS-REFERENCE TO RELATED APPLICATIONS

This application is a U.S. National Phase application of International Application PCT/US2022/015525, filed Feb. 7, 2022, which application claims priority from U.S. Provisional Patent Application Ser. No. 63/146,693, filed Feb. 7, 2021, hereby incorporated in their entireties.

### STATEMENT REGARDING FEDERALLY SPONSORED RESEARCH OR DEVELOPMENT

This invention was made with government support under Grant No. CA116548 awarded by the National Institutes of Health. The government has certain rights in the invention.

### INCORPORATION OF SEQUENCE LISTING

The sequence listing that is contained in the file named “P35049W000_ST25,” which is 98,856 bytes (measured in operating system MS-Windows), recorded on Feb. 7, 2022, is filed herewith and incorporated herein by reference.

### FIELD OF THE INVENTION

The present disclosure relates to engineering NKT cells to preserve the potential of the cells to be expanded ex vivo by culture by antigen challenge and to preserve the central memory gene expression signature of NKT cells. The present disclosure further relates to engineering NKT cells for in vivo persistence in cancer therapy.

### BACKGROUND OF THE INVENTION

Type-I NKT cells (NKTs) are an evolutionary conserved subset of innate lymphocytes that express invariant TCRα-chain Vα24-Jα18 and react to self- or microbial-derived glycolipids presented by monomorphic HLA class-I like molecule CD1d (Gene ID 912) (Porcelli et al. Analysis of T cell antigen receptor (TCR) expression by human peripheral blood CD4-8− alpha/beta T cells demonstrates preferential use of several V beta genes and an invariant TCR alpha chain. J. Exp. Med. 1993; 178(1):1-16); Lantz and Bendelac, “An invariant T cell receptor alpha chain is used by a unique subset of major histocompatibility complex class I-specific CD4+ and CD4-8− T cells in mice and humans,” J Exp. Med. 1994; 180(3): 1097-1106; Bendelac A, Lantz O, Quimby M E, Yewdell J W, Bennink J R, Brutkiewicz R R. CD1 recognition by mouse NK1+ T lymphocytes. Science 1995; 268(5212):863-865; Kim E Y, Lynch L, Brennan P J, Cohen N R, Brenner M B. The transcriptional programs of iNKT cells. Semin. Immunol. 2015; 27(1):26-32).

Global transcriptional profiling studies demonstrate that NKTs, though they share properties with T and NK cells, are a distinct population of lymphocytes (Cohen et al., 2013). Both in mice and humans, NKTs diverge from conventional T cells at the stage of CD4+CD8+ (double positive, DP) thymocytes (CD8, Gene ID 925). Unlike conventional T cells, which are positively selected by thymic epithelial cells, NKTs are selected by CDld-expressing DP thymocytes (Gapin L, Matsuda J L, Surh C D, Kronenberg M. NKT cells derive from double-positive thymocytes that are positively selected by CD1d. Nat. Immunol. 2001; 2(10):971-978). The expression of promyelocytic leukemia zinc finger transcription factor (PLZF) immediately after positive selection enables intrathymic expansion and effector/memory-like differentiation of NKTs (Savage A K, et al. The transcription factor PLZF directs the effector program of the NKT cell lineage. Immunity. 2008; 29(3):391-403).

Peripheral NKTs are long-lived lymphocytes and their post-thymic maintenance largely depends on slow IL-15-mediated homeostatic proliferation (Matsuda J L, et al. Homeostasis of V alpha 14i NKT cells. Nat. Immunol. 2002; 3(10):966-974; Baev D V, et al. Distinct homeostatic requirements of CD4+ (Gene ID 920) and CD4− subsets of Valpha24-invariant natural killer T cells in humans. Blood 2004; 104(13):4150-4156). In human peripheral blood, NKTs are divided into two major functional subsets based on CD4 expression: CD4+ and CD4− (mostly CD8/CD4-double negative, DN) (Lee et al., Distinct functional lineages of human V(alpha)24 natural killer T cells. J Exp. Med. 2002; 195(5):637-641). The CD4+ subset is highly enriched in neonate NKTs and undergoes fewer homeostatic divisions compared with the CD4-subset in adults (Baev et al. 2004 supra), suggesting that CD4+ NKTs could contribute to the long-term persistence of adoptively transferred therapeutic NKTs under certain conditions. However, ex vivo expansion of human NKTs in response to antigenic stimulation, e.g. with a-Galactosylceramide (aGalCer), produces similar numbers of CD4+ and DN NKTs. NKTs also exhibit an NK-like linear differentiation with acquisition of CD161 and then CD56 expression. Like in conventional T cells, the expression of CD56 is associated with terminal differentiation and the loss of proliferative potential (Loza et al., NKT and T cells: coordinate regulation of NK-like phenotype and cytokine production. Eur. J. Immunol. 2002; 32(12):3453-3462).

The potential importance of NKTs for tumor immunity and immunotherapy has been demonstrated in multiple models of cancer in mice and in early stage clinical trials in cancer patients (McEwen-Smith et al., The regulatory role of invariant NKT cells in tumor immunity. Cancer Immunol. Res. 2015; 3(5):425-435; Dhodapkar M V. Harnessing human CD Id restricted T cells for tumor immunity: progress and challenges. Front Biosci. 2009; 14:796-807; ExleyMA, Nakayama T. NKT-cell-based immunotherapies in clinical trials. Clin. Immunol. 2011; 140(2): 117-118; Motohashi S, Okamoto Y, Yoshino I, Nakayama T. Anti-tumor immune responses induced by iNKT cell-based immunotherapy for lung cancer and head and neck cancer. Clin. Immunol. 2011; 140(2):167-176; Yamasaki K, et al. Induction of NKT cell-specific immune responses in cancer tissues after NKT cell-targeted adoptive immunotherapy. Clin. Immunol. 2011; 138(3):255-265; Taniguchi et al., Discovery of NKT cells and development of NKT cell-targeted anti-tumor immunotherapy. Proc. Jpn. Acad. Ser. B Phys. Bio. Sci. 2015; 91(7):292-304). In contrast to conventional T cells, NKTs effectively traffic to the tumor site and can mediate anti-tumor responses either via direct killing of CDld+ tumor cells, inhibition of tumor-supportive macrophages, or trans-activation of NK cells (Metelitsa LS. Anti-tumor potential of type I NKT cells against CD ld-positive and CD ld-negative tumors in humans. Clin. Immunol. 2011; 140(2): 119-129).

Several studies have revealed strong positive associations between the numbers of tumor-infiltrating or circulating NKTs and improved disease outcome in patients with diverse tumor types (Dhodapkar, supra; Metelitsa L S, et al. Natural killer T cells infiltrate neuroblastomas expressing the chemokine CCL2. J. Exp. Med. 2004; 199(9):1213-1221; Tachibana T, et al. Increased intratumor Valpha24-positive natural killer T cells: a prognostic factor for primary colorectal carcinomas. Clin. Cancer Res. 2005; ll(20):7322-7327; Moiling P W, et al. Low levels of circulating invariant natural killer T cells predict poor clinical outcome in patients with head and neck squamous cell carcinoma. J. Clin. Onco. 2007; 25(7):862-868; Cariani E, et al. Immunological and molecular correlates of disease recurrence after liver resection for hepatocellular carcinoma. PLoS. One. 2012; 7(3):e32493.). Conversely, tumor progression is often accompanied by a decrease in NKT-cell number or functional activity (16), or the downregulation of CDTd expression on malignant cells (Dhodapkar M V, et al. A Reversible Defect in Natural Killer T Cell Function Characterizes the Progression of Premalignant to Malignant Multiple Myeloma. J. Exp. Med. 2003; 197(12):1667-76.). To counteract these tumor escape mechanisms, methods were developed to expand primary human NKTs to clinical scale ex vivo and to redirect their cytotoxicity against tumor cells via transgenic expression of chimeric antigen receptors (CARs) (Heczey et al., Invariant NKT cells with chimeric antigen receptor provide a novel platform for safe and effective cancer immunotherapy. Blood 124(18):2824-2833 (2014)). Similar to the observations reported in CAR T-cell clinical trials (Kalos and June, 2013; Dotti G et al., Design and development of therapies using chimeric antigen receptor-expressing T cells. Immunol. Rev. 2014; 257(1):107-126), there is a strong correlation between the anti-tumor efficacy and in vivo persistence of CAR NKT-cell products in a xenogenic tumor model (Heczey et al., 2014). However, the mechanisms that govern ex vivo expansion and subsequent in vivo persistence of human NKTs remain largely unknown, impeding rational design of NKT cell-based cancer immunotherapy.

Reports demonstrating that CD62L+(Selectin L, Gene ID 6402) central memory conventional T cells have stem cell properties and superior therapeutic activity in cell therapy products (Graef P, et al. Serial transfer of single-cell-derived immunocompetence reveals sternness of CDS(+)central memory T cells. Immunity. 2014; 41(1): 116-126; Wang X, et al. Phenotypic and functional attributes of lentivirus-modified CD19-specific human COB+ central memory T cells manufactured at clinical scale. J Immunother. 2012; 35(9):689-701; Sommermeyer D, et al. Chimeric antigen receptor-modified T cells derived from defined COB and CD4 (Gene subsets confer superior antitumor reactivity in vivo. Leukemia 2015) were followed by the examination of CD62L+ NKT cells in ex vivo culture.

International Patent Publication No. WO 2016/172372, published Oct. 27, 2016, demonstrated that the CD62L+ subset of NKT cells is required for NKT cell ex vivo expansion and in vivo persistence. Further, when engineered to express CD19-specific CAR (CAR.CD19), CD62L+ but not CD62L− CAR.CD19 NKTs produced sustained tumor regression in a B-cell lymphoma model in NSG mice. CD62L+ NKTs could be maintained during ex vivo expansion when provided with certain costimulatory ligands such as co-stimulatory artificial antigen-presenting cells (aAPC) that can be used to generate NKTs and CAR-NKTs with superior therapeutic activity in patients with cancer, for example. These advances provided robust methods to expand primary human NKTs to clinical scale ex vivo and to redirect their cytotoxicity against tumor cells via transgenic expression of chimeric antigen receptors, but challenges remain. There continues to be a need for improved methods of expansion ex vivo. Further challenges relate to retaining their persistence in vivo when the CAR NKT cells are used therapeutically.

Here it is shown that CD62L+ NKT cells share similar gene expression with central memory CD4+ conventional T cells. It is also shown that Wnt signaling plays a critical role in both driving and maintaining CD62L+ expression in NKT cells and exploiting these observations solve a number of problems with developing NKT cells as therapeutic agents. As provided below, expression of activators of Wnt signaling during ex vivo culture and expansion leads to increased CD62L+ expression. Engineered NKT cells having activated Wnt signaling exhibit reduced exhaustion and can undergo multiple rounds of expansion. Engineered NKT cells having activated Wnt signaling further preserve the central memory cell expression profile after antigen challenge. Importantly, engineered NKT cells having activated Wnt signaling provide for in vivo persistence and improved clinical effectiveness.

### SUMMARY OF THE INVENTION

The present disclosure provides for, and includes, genetically engineered NKT cell, and populations thereof, comprising an expression construct for a transcriptional activator in the Wnt signaling pathway.

The present disclosure provides for, and includes, populations of cells comprising a plurality of genetically engineered NKT cells comprising expression construct encoding a protein sequence for a transcriptional activator in the Wnt signaling pathway.

The present disclosure provides for, and includes, engineering NKT cells to express at least a protein coding sequence comprising a transcriptional activator in the Wnt signaling pathway; and culturing the engineered NKT cells to prepare a population of genetically engineered NKT cells with persistent expansion potential.

The present disclosure provides for, and includes, engineering NKT cells to express a (a) a chimeric antigen receptor (CAR) coding sequence comprising an ectodomain sequence, a transmembrane domain sequence, and an endodomain sequence and (b) a sequence encoding a protein sequence for a transcriptional activator in the Wnt signaling pathway.

The present disclosure also provides for, and includes, a method of maintaining NKT cell expansion potential comprising the steps of transfecting or transducing cultured NKT cells with a protein expression construct comprising a transcriptional activator in the Wnt signaling pathway; and culturing the engineered NKT cells to prepare a population of NKT cells with persistent expansion potential.

Also included and provide for by the present disclosure are chimeric antigen receptor expression constructs comprising a chimeric antigen receptor coding sequence and a coding sequence a transcriptional activator in the Wnt signaling pathway.

The present disclosure further provides for, and includes, a method of reducing NKT cell exhaustion in an NKT cell population comprising the steps of engineering NKT cells to express at least a protein coding sequence comprising a transcriptional activator in the Wnt signaling pathway culturing the engineered NKT cells to prepare a population of NKT cells with reduced NKT cell exhaustion.

The present disclosure further provides for, and includes, a method of reducing NKT cell exhaustion in an NKT cell population comprising the steps of transfecting or transducing cultured NKT cells with an expression construct comprising (a) a chimeric antigen receptor (CAR) coding sequence comprising an ectodomain sequence, a transmembrane domain sequence, and an endodomain sequence and (b) a sequence encoding a protein sequence for a transcriptional activator in the Wnt signaling pathway.

The present disclosure further provides for, and includes, a method of maintaining central memory NKT cells from loss after antigen challenge, comprising the steps of engineering cultured central memory NKT cells with a protein expression construct comprising a transcriptional activator in the Wnt signaling pathway; and culturing said engineered central memory NKT cells to prepare engineered central memory NKT cells with resistance to loss after antigen challenge.

The present disclosure further provides for, and includes, methods of maintaining a central memory characteristic in NKT cells, comprising the steps of engineering an NKT cell with an expression construct to express a sequence encoding a protein that is a transcriptional activator of the Wnt signaling pathway culturing the engineered NKT cell to express the transcriptional activator and preparing a population of genetically engineered NKT cells that retain proliferative ability and cytotoxicity of the central memory NKT cell characteristic after repeated challenge.

The present disclosure further provides for, and includes, methods of maintaining a central memory characteristic in NKT cells, comprising the steps of engineering an NKT cell with an expression construct comprising (a) a chimeric antigen receptor (CAR) coding sequence comprising an ectodomain sequence, a transmembrane domain sequence, and an endodomain sequence and (b) a sequence encoding a protein sequence for a transcriptional activator in the Wnt signaling pathway and preparing a population of genetically engineered NKT cells that retain proliferative ability and cytotoxicity of the central memory NKT cell characteristic after repeated challenge.

The present disclosure further provides for, and includes, a method of preparing CD62L(+) NKT cells for therapeutic use comprising obtaining NKT cells for ex vivo culture and culturing the NKT cells in the presences of aGalCer pulsed PBMCs or aAPCs loaded with aGalCer and an added Wnt signaling pathway activator selected from the group consisting of a Wnt ligand, a GSK3β inhibitor, IL-21, TWS119, small molecule activators of the Wnt pathway, or a combination thereof.

### BRIEF DESCRIPTION OF THE DRAWINGS

The present disclosure is disclosed with reference to the accompanying drawings, wherein:

FIG. 1 presents a Gene-set enrichment analysis (GSEA) plot showing enrichment for a central memory CD4+ T cell signature (Abbas et al., 2009) partitioned into CD62L+ NKTs showing that CD62L+ NKTs have a central memory-like RNA expression profile. (Abbas et al., 2009)

FIG. 2 is a representative histogram from one of seven donors (a) and mean±SEM of the percentage of LEF1+ cells for all donors (b) (n=7, ***P<0.001, paired Student's t test) validating key findings from expression deconvolution.

FIG. 3 is representative plots of intracellular flow cytometry from three donors showing co-expression of LEF1 and TCF1 in ex vivo-expanded NKTs. NKTs are gated into CD62L− (a) and CD62L+ (b) populations.

FIG. 4 is a representative dot plot of intracellular flow cytometry of a donor gating the cells into CD62L− LEF1−, CD62L+ LEF1− and CD62L+ LEF1+ populations (a), a representative histogram from one of seven donors of TIM-3 expression is shown in (b), and mean±SEM of TIM-3 MFI for all donors in (c) (n=7, *P<0.05, ns: not significant, one-way ANOVA with Sidak's post-test).

FIG. 5 is a summary of the results of four donors for the expression of Wnt target genes measured by quantitative PCR and calculated by the ddCt method of magnetically sorted CD62L+ and CD62L− subsets. Axin2 (a), Myc (b) and TCF7 (c).

FIG. 6 is a representative dot plots of intracellular flow cytometry showing transduction and Wnt activity in NKT cells transduced with the 7-TGC reporter (a) on day 12 after ex vivo stimulation. Dot-plots of non-transduced (b), 7-TGC-transduced (c) and 7-TGC-transduces NKTs treated with 10 μM Wnt activator TWS119 overnight (c) are shown.

FIG. 7 is a representative histogram of CD62L expression in mCherry+ NKTs gated into GFP− and GFP+ subsets, from one of four donors (a) and mean CD62L+ percentage for all donors (b) (n=4, **P<0.01, Student's t test) for are shown. Each symbol denotes an individual donor.

FIG. 8 is a plot of summary of summary of three independent experiments, each with an individual donor of gene expression of mRNA expression in NKTs expanded ex vivo for 10 days and measured by quantitative RT-PCR. GAPDH (Gene ID 2597) and CD19 (Gene ID 930) present positive and negative expression, respectively. Genes with Ct values that fall within the range of GAPDH and CD19 were considered significantly expressed. Wnt receptors (FRZ1 to FRZ10, (a), co-receptors (LRP5,6), and ligand (Wnt1, Wnt2, Wnt3a, Wnt6, and Wnt8a) are shown.

FIG. 9 is a plot of summary of summary of three independent experiments, of RNA isolated from K562-based aAPCs followed by quantitative PCR showing that Wnt ligand expression (Wnt1, Wnt2, Wnt3a, Wnt6, and Wnt8a) is not detected. GAPDH and CD19 present positive and negative expression, respectively.

FIG. 10 is a representative histogram from one of three donors (a) and mean of CD62L+ percentage for all donors (b) (n=3, *P<0.05, Student's t test) showing NKTs treated with 3 μM of Wnt inhibitor ICG-001 after stimulation with aGalCer-pulsed autologous PBMCs starting at day 0 for three days. CD62L expression is examined on day 12 after antigenic stimulation. Each symbol denotes an individual donor.

FIG. 11 is a representative histogram from one of three donors (a) and mean of CD62L+ percentage for all donors (b) (n=3, *P<0.05, Student's t test) showing NKTs stimulated as shown in FIG. 10 but treated with three separate doses of PBS or 500 ng/mL Wnt3a given on day 0, 3 and 7 after antigenic stimulation. CD62L expression is examined by flow cytometry on day 12 after antigenic stimulation.

FIG. 12 is a representative histogram from one of six donors (a) and mean of CD62L+ percentage for all donors (b) (n=6, **P<0.01, Student's t test) showing NKTs stimulated as shown in FIG. 10, but treated with one dose of DMSO or 5 μM TWS119 on day 7 after antigenic stimulation. CD62L expression is examined by flow cytometry on day 12 after antigenic stimulation. Each symbol denotes an individual donor.

FIG. 13 is a representative histogram showing the frequency of CD62L(+) NKTs stimulated with aGalCer-pulsed autologous PBMCs and treated with three doses of PBS or 500 ng/ml Wnt3a in early (days 0,1,2), late (days 6,7,8) or throughout (days 0,3,8) expansion. CD62L expression is examined by flow cytometry on day 12 after antigenic stimulation.

FIG. 14 is a representative histogram of one of three donors showing flow cytometry results of NKT cells at the end of expansion and culture with IL-2 or IL-2/IL-21 for 12 days following primary stimulation. In each group, PBS or 500 ng/ml Wnt3a treatment is administered on days 0, 1, and 2 after antigenic stimulation.

FIG. 15 is a representative histogram of one of three donors showing flow cytometry results for CD62L expression of NKT cells cultured as shown in FIG. 14 and further treated with DMSO or 5 μM TWS119 on day 7 after antigenic stimulation.

FIG. 16 is a representative histogram from one of three donors (a) and mean±SEM of CD62L+ percentage for all donors (b) (n=3, **P<0.01, ns: not significant, Student's t test) for NKTs electroporated with Cas9 with or without guide RNA targeting LEF1 on day 10 of ex vivo expansion. CRISPR/Cas9-mediated LEF1 KO is evaluated by intracellular flow cytometry on day 3 after electroporation. NKTs are gated into CD62L- and CD62L+ populations and LEF1 expression is analyzed.

FIG. 17 is a representative histogram from one of three donors (a) and mean±SEM of CD62L+ percentage for all donors (b) (n=3, **P<0.01; ns: not significant, paired Student's t test) in electroporated NKTs before antigenic stimulation at day 7 after electroporation.

FIG. 18 is a representative histogram from one of three donors (a) and mean±SEM of CD62L+ percentage for all donors (b) (n=3, **P<0.01; ns: not significant, paired Student's t test) in electroporated NKTs as shown in FIG. 17 at Day 10 after antigenic stimulation.

FIG. 19 are representative dot plots from one of three donors transduced with a gammaretroviral construct that overexpresses the long isoform of LEF1 (Ref SEQ ID: NM_016269.5) (GFP.LEF1), or a GFP.FFLuc construct, two days after secondary stimulation using αGalCer-pulsed aAPCs. On day 12 of expansion, GFP expression was evaluated by flow cytometry and LEF1 expression was analyzed separately by intracellular flow cytometry.

FIG. 20 is a representative histogram from one of two donors analyzed by MitoTracker staining and flow cytometry in GFP.FFLuc- and GFP.LEF1-transduced NKTs as shown in FIG. 19.

FIG. 21 is a graph of oxygen consumption rate (OCR) of GFP.FFLuc- and GFP.LEF1-transduced NKTs was measured by Seahorse assay under basal conditions and in response to oligomycin, FCCP, and rotenone and antimycin A (Rot/Ant). Results are representative from one of two donors tested.

FIG. 22 is a graph presenting the fold change of NKT cell number for NKTs transduced with GFP.FFLuc and GFP.LEF1 after repeated challenge with CD1d+ J32 leukemia cells at a 1:1 ratio every three days. Fold change in expansion for each cycle is shown.

FIG. 23 is a graph presenting the results of a seventh cycle of repeated killing assay as shown in FIG. 22, NKTs are challenged with J32 cells at a 1:50 ratio for three days. Tumor cell number is determined using counting beads and flow cytometry. Tumor reduction percentage is shown.

FIG. 24 is a graph presenting the results of a fifth cycle of repeated killing assay as in FIG. 22. NKTs are isolated from antigen stimulation and rested for a total of six days. TIM-3 expression in relation to GFP is examined by flow cytometry. Representative dot plots from one of four donors (a) and mean TIM-3 MFI for all donors (b) (n=4, ***P<0.001, ns: not significant, one-way ANOVA and Sidak's post-test). are shown. Each symbol denotes an individual donor.

FIG. 25 is a representative histogram from one of four donors (a) and mean CD62L percentage for all donors (b) (n=4, P=0.07, one-way ANOVA and Sidak's post-test) are shown. Each symbol denotes an individual donor.

FIG. 26 is a graph of enrichment of transduced cells monitored by GFP expression using flow cytometry of NKTs repeatedly challenged as described in FIG. 22.

FIG. 27 presents diagrams of exemplary chimeric antigen receptors according to embodiments of the present disclosure.

FIG. 28 presents diagrams of exemplary CAR constructs encoding co-expression of LEF1. The LEF1 coding sequences are inserted after a 2A sequence downstream of the CD28 (Gene ID 940) or 4-1BB endodomain, or IL-15 as indicated.

FIG. 29 presents dot plots of NKTs transduced with parental or LEF1-containing CAR.GD2 constructs two days after secondary stimulation with αGalCer-pulsed aAPCs and surface expression of CAR.GD2 and intracellular expression of LEF1 determined by flow cytometry at day 12 of expansion. Representative dot plots show LEF1 expression in relation to CAR expression from one of two donors.

FIG. 30 presents dot plots of representative results of NKTs transduced with parental or LEF1-containing expression construct of an after one, two, or seven challenges by J32 tumor cells at a 1:1 cell to cell ratio. LEF1-containing NKT cells persist at high levels after 7 cycles while the control parental NKT cells showing that LEF1 over overexpression promotes late-stage NKT expansion after repeat tumor cell killing.

FIG. 31 presents the experimental design of a tumor cell killing challenge using an effector to target ratio of 1:50.

FIG. 32 shows the representative results of NKT expansion after NKT challenge of tumor cells at a 1:50 ratio as shown in FIG. 31. LEF1 expressing NKT cells retain the ability to expand and kill tumor cells. In contrast, GFP expressing control cells fail to expand and do not exhibit tumor cell reduction.

FIG. 33 presents a graphical representation of the results shown in FIG. 32.

FIGS. 34A and B presents representative results of gene expression analysis ten days after secondary stimulation. Differentially expressed genes (DEGs) of interest are grouped by shared phenotype/function. FIG. 34A, panel a, presents LEF1 overexpression mediated differential gene expression in NKTs. The results are presented below in Table 4. FIG. 34A, panel b presents the gene expression results as a heat maps show fold change in expression (LEF1/FFLuc). FIG. 35B, panel c presents a GSEA plot showing a central memory T cell signature according to the method of Jeffrey et al., “Positive regulation of immune cell function and inflammatory responses by phosphatase PAC-1,” Nat. Immunol. 7)3_:274-283 (2006). FIG. 35B, panel d presents a GSEA plot showing enrichment for a CD8 T cell signature with lower exhaustion levels according to the method of Duraiswamy et al., “Phenotype, function, and gene expression profiles of programmed death-1(hi) CD8 T cells in healthy human adults,” J. Immunol. 186(7):4200-4212 (2011) in LEF1-overexpressing NKTs.

FIG. 35A-D, panels a to g, present the design of CAR-LEF1 constructs and the characterization of transfected donor NKT cells. FIG. 35A, panel a presents a schematic of an exemplary design of a CAR LEF1 constructs containing the anti-GD2 14g2a scFv, CD8 hinge and transmembrane domains, 4-1BB co-stimulatory domain, and CD3 zeta domain, with LEF1 following a 2A sequence (CAR-LEF1) or without (CAR). FIG. 35A, panel b presents a representative dot plot showing LEF1 expression relative to CAR expression from one of two donors. FIG. 35A, panel c present a graph showing NKT cell number determined by trypan blue exclusion assay and showing that LEF1 incorporation improves numeric expansion of CAR-NKTs. FIG. 35B, panel d presents a representative histogram of the frequency of CD62L expression after 10 days of expansion and presenting the mean of six representative donors. FIG. 35B, panel e presents a representative histogram of the frequency of TIM-3 positive cells after expansion and presenting the mean of six representative donors. FIG. 35C, panel f presents a graphical analysis of the short term cytotoxicity of CAR-NKTs. Luciferase-transduced GD2+ CHLA-255 cells co-cultured with CAR or CAR-LEF1 NKTs. The results of a representative donor are presented. FIG. 35C, panel g presents a graphical analysis of the production of of effector cytokines from CAR-NKTs. Representative results of one of two donors is shown.

FIGS. 36A and B present the results of the superior effect of CAR-LEF1 NKTs in the control of tumors in mice in vivo. FIG. 36A shows the bioluminescence imaging of expanded NKTs from a non-transduced (NT) control group, a CAR.GD2 (CAR), or CAR.GD2-LEF1 (CAR-LEF1) transduced cells. Tumor growth is monitored using bioluminescence imaging once per week. FIG. 36B shows a graphical representation of the survival of the test mice generated using the Kaplan-Meier method.

Corresponding reference characters indicate corresponding parts throughout the several views. The example(s) set out herein illustrate(s) [one/several] embodiment(s) of the present disclosure but should not be construed as limiting the scope of the present disclosure in any manner.

### DETAILED DESCRIPTION

The present application is directed to genetically modified natural killer T cells (NKT cells). NKT cells are a distinct cell type that share some features of both T and NK cells but are distinct from both conventional T cells and also NK cells. NKT cells have divergent development from conventional T cells and NK cells and different functions driven by a unique set of transcriptional regulators. See Kronenberg M, Gapin L. The unconventional lifestyle of NKT cells. Nat. Rev. Immunol. 2002; 2(8):557-568; Godfrey, J C I, 2004, Cohen N R, et al. Shared and distinct transcriptional programs underlie the hybrid nature of iNKT cells. Natdmmunol. 2013; 14(1):90-99.). Godfrey et al., identify transcription factors, signal-transduction factors, cell surface molecules, cytokines, and other factors that selectively influence NKT cell development reflecting the unique programming associated with the NKT cell lineage. (Godfrey et al., “Raising the NKT cell family,” Nat. Immunol., 11(3):197-206 (2010) (“Godfrey et al.”) hereby incorporated by reference in its entirety. See also Engel and Kronenberg, “Transcriptional control of the development and function of Vα4i NKT cells,” Current Topics in Microbiology and Immunology, Volume 381, 2014). Many transcription factors and signaling molecules that affect NKT cells differentiation in the thymus do not affect other conventional T cell populations that develop there. As used throughout the present disclosure, the term “T cell” is limited to conventional T cells that are distinguishable from NKT cells. These differences result in different responses to stimuli and genetic changes such as engineered gains and losses of gene expression that make results in non-NKT cells unpredictable.

NKT cells are distinguishable based on whole genome transcription analysis and are equally distant from conventional and NK cell lineages. See Cohen et al. supra. Conventional T cells, also known as T lymphocytes, are an important cell type with the function of fighting pathogens and regulating the immune response. Two hall marks of these cells are expression of an antigen receptor encoded by segments of DNA that rearrange during cell differentiation to form a vast array of receptors. A number of cells fall within this generic definition of a T cell, for example: T helper cells (CD4+ cells) including the sub-types TH1, TH2, TH3, TH17, TFH; cytotoxic T cells (mostly CD8+ cells, also referred to a CTLs); memory T cells (including central memory T cells, effector memory T cells, and resident memory T cells); regulatory T cells, and mucosal associated invariant T cells. Cell surface markers of T cells include the T cell receptor and CD3. Generally T cells do not express CD56 (i.e. are CD56 negative).

NK cells and NKT cells are CD56+. In humans NK cells usually express the cell surface marker CD56, CD161, CD1 Ib, NKp46, NKp44, CD158 and IL-12R. NK cells express a limited repertoire of receptors with an entirely different structure, some of which are also found on NKT cells. Most NK receptors are not highly conserved comparing humans and rodents. NK cells express members of the family of killer-cell-immunoglobulin-like receptors (KIRs), which can be activating or inhibiting, as well as receptors that are members of the lectin (carbohydrate-binding) family of proteins such as NKG2D and CD94NKG2A/C. KIRs are not expressed on NKT cells. NK cells are activated by a number of cell surface receptors, such as KIRs in humans or Ly49 in mice, natural cytotoxic receptors (NCRs), NKG2D and CD94:NKG2 heterodimers. In addition cytokines and chemokines, such as IL-12, IL-15, IL-18, IL-2 and CCLS, play a significant role in NK cell activation.

NKT cells generally can be identified as CD3+CD56+ cells and express a T cell receptor. NKT cells express a T cell receptor and CD3 chains like T cells, but also have markers such CD56 and CD161, like NK cells. Having said that, it is now commonly accepted by experts that they are a distinct lineage of cells. That is they are very different from other T cells and their behavior and properties cannot be predicted from analysis of other T cells, nor are they NK cells. NKT cells are completely different cells to conventional T cells and to NK cells. Due to the unique properties of the NKT cell lineage, observations made with other populations of lymphocytes, such as T cells, NK cells, and B cells, may not predict functional consequences of NKT cell activation.

NKT cells can be identified from other cell types including CD4 T cells, CD8 T cells, regulatory T cells, γδ T cells, B cells, NK cells, monocytes and dendritic cells based on the expression of cell surface markers. See Park et al., “OMIP-069: Forty-Color Full Spectrum Flow Cytometry Panel for Deep Immunophenotyping of Major Cell Subsets in Human Peripheral Blood,” Cytometry PartA 97A:1044-1051 (2020); Hertoghs et al., OMIP-064: A 27-Color Flow Cytometry Panel to Detect and Characterize Human NK Cells and Other Innate Lymphoid Cell Subsets, MAIT Cells, and γδ T Cells, Cytometry Part A 97A:1019-1023 (2020); Sahir et al., Development of a 43 color panel for the characterization of conventional and unconventional T-cell subsets, B cells, NK cells, monocytes, dendritic cells, and innate lymphoid cells using spectral flow cytometry, Cytometry 2020:1-7.

NKT cells are divided into two main types, Type I and Type II. The most significant form of NKT cells, known as type I NKT cells or invariant NKT cells (“iNKT”), have an invariant T cell receptor alpha chain (Vα4i mouse or Vα24i human). Type I NKT (iNKT) cells can be readily detected by the binding of CD1d-based tetramers loaded with αGalCer analogs. The form of the antigen receptor is a limited repertoire due to an invariant alpha chain paired with one of a relatively small number of beta chains. inhibition, or therapeutic use. The antigens recognized by this invariant receptor are glycolipids, for example those found in bacterial cells. The invariant receptor recognizes alpha-galatosylceramide (a-GalCer) a glycolipid originally derived from marine sponges. This compound is similar to microbial glycolipids, and it is now generally assumed to be derived from a microbial symbiont associated with the sponge. NKT cells require antigen presented on a molecule CD1d.

Type II NKT cells also require antigen presentation from CD1d but have a more diverse but still limited TCR repertoire. Type II NKT cells express low levels of the transcription factor PLZF. While Type I NKT cells only recognize a-GalCer, Type II NKT cells recognize sulfatide, lyso-sulfatide, Lyso-PC and Lyso-GL1. Type II NKT cells are more prevalent in humans, but less prevalent in mice. See Dhodpkar and Kumar, “Type II NKT Cells and Their Emerging Role in Health and Disease,” J Immunol. 198(3):1015-1021 (2017).

Two pathways are known for NKT cell activation. NKT cells respond stimulation through their T cell receptor via antigen presented on CD1d molecules. This does not depend upon the involvement of a CD4 or CD8 co-receptor to generate a TCR signal, and the response of these cells is somewhat less dependent on a co-stimulatory signal. In addition, a mechanism for activation of NKT cells exists in the absence of antigen engaging the T cell receptor, via innate inflammatory stimuli, such as IL-12 and IL-18. Once activated T cells are found in the peripheral blood. Similarly NK cells are found in the peripheral blood. In contrast the majority of NKT cells are found in tissues and they migrate away from peripheral blood to the site of tumors, for example as mediated via a two-step process involving CCR2 and CCR6. The mechanisms involved in this migration are specific to NKT cells and not general mechanisms that apply to other lymphocytes.

iNKT cells are readily distinguishable from other T-cell types. See Table 1. Only a small fraction of expanded T cells (a subset of CD4 T cells) can produce tumor-protective Th2 cytokines (IL-4, IL-5, IL-13, IL-10) upon activation either via the T cell receptor (TCR). The majority of T cells (including all CD8+ T cells) and all NK cells produce only anti-tumor Th1 cytokines (i.e. IFN-gamma, GM-CSF, TNF-alpha). In contrast, NKT cells simultaneously produce Th1 and Th2 cytokines.” Depending on the balance of Th1 and Th2 cytokines produced after T cell receptor (TCR) activation, NKT cells can either activate or suppress the immune response. Thus NKT cells have an intriguing paradoxical dual function of immune activation and immune suppression. In contrast other immune cells usually have one primary function, for example fighting pathogens, whilst other subsets of cells are dedicated to regulating the immune response.

TABLE 1
Distinguishing features of iNKT cells

| T CELLS | iNKT CELLS |
|---|---|
| TCR specificity varies | TCR specificity does not vary |
| TCR binds peptides presented | TCR binds certain glycolipids, for example |
| on MHC molecules | natural products and derivatives from |
| | bacterial cell walls, presented on CD1d |
| | TCR/MHC/peptide complex formed TCR has |
| | unique docking strategy with CD1d |
| Part of the reactive immune system | Part of the innate immune system |
| Take time to react to a “threat” | React very quickly to a “treat” |
| Involved in tissue rejection | Not involved in tissue rejection |
| Tolerant to self-antigens | Can react to self-antigens |
| Activated by antigen binding | Is generally activated by antigen binding but |
| | is not activated by antigen binding in the |
| | hypoxic tumor microenvironment. May not |
| | be activated by binding in a “second” encounter. |
| Non-specifically activated by | Can be activated by the cytokines IL-12 and |
| anti-CD3 agonistic antibody | IL-18 |
| Primarily located in blood | Generally resident in tissue |
| Do not co-located with tumor | Co-located with tumor associated macrophage |
| associated macrophages | in hypoxic tumor microenvironment |
| Does not migrate to tumor | Migrates to the tumor microenvironment |
| | via a unique CCR2 and CCR6 mechanism |
| Have a clear hierarchy of naïve- | CD4+ and CD4− NKT are unique |
| central-effector differentiation | differentiation states |

Developmental pathway is distinct for the two cell types
In vitro stimulation/culture of the T cell and NKT cells require different protocols

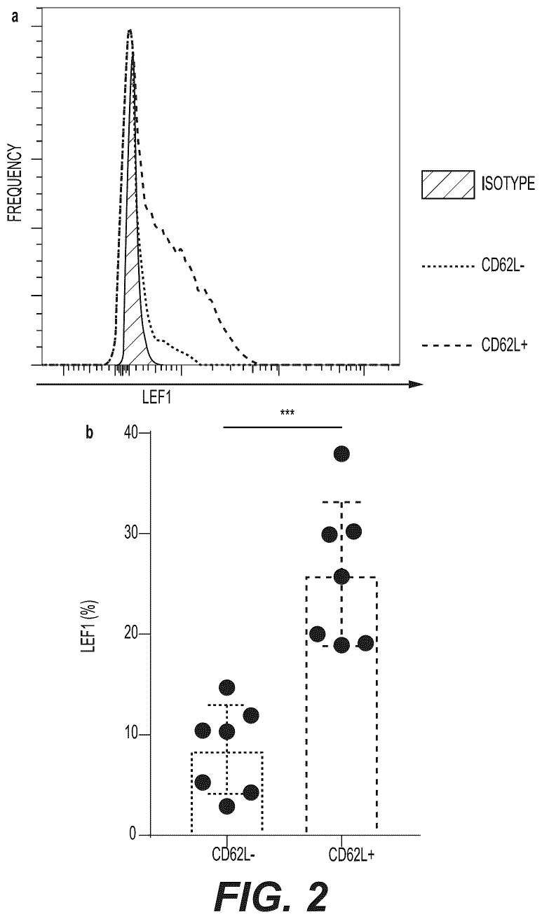

NKT cells also develop in the thymus, however, the positive selection of Type I NKT cells is mediated by CD Id positive thymocytes. NKT cells are also subject to negative selection by dendritic cells. See Godfrey et al., at FIG. 2 summarizing the development and maturation of T cells and NKT cells in the thymus.

Unless defined otherwise, all technical and scientific terms used herein have the meaning commonly understood by a person skilled in the art to which this invent ion belongs. The following references provide one of skill with a general definition of many of the terms used in this invention: Singleton et al., Dictionary of Microbiology and Molecular Biology (2nd ed. 1994); The Cam bridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein the term “about” refers to plus/minus 10%.

The terms “comprises” ˜, “ccomprising” ˜, “includes”, “including”, “˜having” ˜ and their conjugates mean “including but not limited to.”

The term “consisting of” means “including and limited to.”

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a cell” or “at least one cell” may include a plurality of cells, including mixtures thereof.

The terms “comprises”, “comprising”, and are intended to have the broad meaning ascribed to them in U.S. Patent Law and can mean “includes”, “including” and the like.

By “increase” is meant to alter positively by at least 5%. An alteration may be by 5%, 10%, 25%, 30%, 50%, 75%, or even by 100%.

Throughout this application, various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

As used herein, a “genetically engineered natural killer T (NKT)” is an NKT cell that comprises at least one recombinant nucleic acid encoding exogenous protein or a endogenous protein downstream of a non-native promoter. In aspects, genetically engineered NKT cells comprise a recombinant nucleic acid encoding a chimeric antigen receptor. Also provided for, and included, are NKT cells that comprise a recombinant nucleic acid encoding a chimeric antigen receptor and a recombinant nucleic acid encoding a protein sequence for a transcription factor in the Wnt signaling pathway though the two protein encoding sequences can be provided in separate recombinant nucleic acid constructs.

By “endogenous” is meant a nucleic acid molecule or polypeptide that is normally expressed in a cell or tissue.

By “exogenous” is meant a nucleic acid molecule or polypeptide that is not endogenously present in the cell, or not present at a level sufficient to achieve the functional effects obtained when over-expressed. The term “exogenous” would therefore encompass any recombinant nucleic acid molecule or polypeptide expressed in a cell, such as foreign, heterologous, and over-expressed nucleic acid molecules and polypeptides.

By “isolated cell” is meant a cell that is separated from the molecular and/or cellular components that naturally accompany the cell.

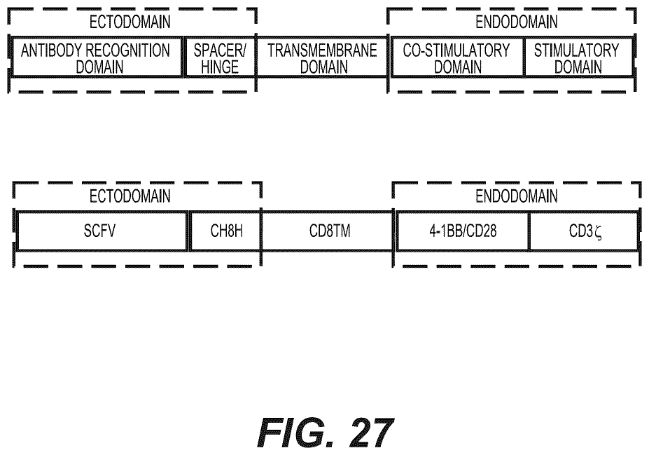

The term “chimeric antigen receptor” or “CAR,” as used herein, refers to an artificial T cell receptor that is engineered to be expressed on an immune effector cell and specifically bind an antigen. A diagram of a generic CAR is shown in FIG. 27a. In aspects, CARs comprise and ectodomain, a transmembrane domain, and an endodomain. In certain aspects, a CAR can comprise an ectodomain and transmembrane domain without an endodomain, but more CARs of the present application include the endodomain and provide for intracellular signaling.

By “receptor” is meant a polypeptide, or portion thereof, present on a cell membrane that selectively binds one or more ligands.

As used herein, the term “ectodomain” refers to the extracellular portion of a CAR and encompasses a signal peptide, an antigen recognition domain, and a spacer or hinge region that links the antigen recognition domain to the transmembrane domain. When expressed, the signal peptide may be removed.

As used herein, an “antigen recognition domain” generally comprises a single chain variable fragment (scFv) specific for a particular cancer antigen. In some aspects, where there are two or more CARs in the same cell, the second CAR may comprise an scFv specific for another particular antigen.

As used herein, the term “single-chain variable fragment” or “scFv” is a fusion protein of the variable regions of the heavy (VH) and light chains (VL) of an immunoglobulin covalently linked to form a VH::VL heterodimer. The heavy (VH) and light chains (VL) are either joined directly or joined by a peptide-encoding linker (e.g., 10, 15, 20, 25 amino acids), which connects the N-terminus of the VH with the C-terminus of the VL, or the C-terminus of the VH with the N-terminus of the VL. The linker is usually rich in glycine for flexibility, as well as serine or threonine for solubility. Despite removal of the constant regions and the introduction of a linker, scFv proteins retain the specificity of the original immunoglobulin. Single chain Fv polypeptide antibodies can be expressed from a nucleic acid including VH- and VL-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754.

As used herein, a “spacer” or “hinge region” is an optional linker portion of the recombinant protein further that is a short peptide fragment between the transmembrane domain and the antibody recognition domain. The spacer or hinge region can be between 1 and 20 amino acids. Examples of hinge regions for the ectodomain include the CH2CH3 region of immunoglobulin, the hinge region from IgG1, and portions of CD3.

As used herein, a “transmembrane domain” is a region of predominantly of nonpolar amino acid residues that when the protein is expressed, traverses the bilayer at least once. Generally, the transmembrane domain is encoded by 18 to 21 amino acid residues and adopts an alpha helical configuration. As used herein, the transmembrane domain may be of any kind known in the art. In aspects the transmembrane domain is although in some cases it is CD28. Other sources include CD3-C, CD4, or CD8. An exemplary combination of an ectodomain is shown in FIG. 27b. Other suitable transmembrane regions can be obtained from CD16, NKp44, NKp46, and NKG2d.

As used herein, the term “endodomain” refers to the intracellular domain of a CAR that provides for signal transmission in a cell. Generally, the endodomain can be further divided into two parts, a stimulatory domain and optionally, a co-stimulatory domain. The co-stimulatory domain is shown to be arranged amino-terminal to the stimulatory in FIG. 27a, but the present specification also provides for an amino terminal stimulatory domain and followed by a co-stimulatory domain when present. The most commonly used endodomain component is CD3-zeta that contains 3 ITAMs and that transmits an activation signal to the NKT cell after the antigen is bound. Other suitable stimulatory domains can be obtained from 2B4 (CD244), TNF receptor superfamily member 9 (Gene ID 3604, e.g., 4-1BB or CD137), Interleukin 21 (IL-21, Gene ID 59067), hematopoietic cell signal transducer (HCST, Gene ID 10870 e.g., DAP10), and transmembrane immune signaling adaptor (TYROBP, Gene ID 7305; DAP12).

The term “tumor antigen” as used herein refers to an antigen (e.g., a polypeptide, glycoprotein, or glycolipid) that is uniquely or differentially expressed on a tumor cell compared to a normal or non-neoplastic cell. With reference to the invention, a tumor antigen includes any polypeptide expressed by a tumor that is capable of being recognized by an antigen recognizing receptor (e.g., CD19, Muc-1) or capable of suppressing an immune response via receptor-ligand binding (e.g., CD47, PD-L1/L2, 87.112).

By “tissue antigen” is meant an antigen (e.g., a polypeptide or glycoprotein or glycolipid) that is uniquely or differentially expressed on a normal or non-neoplastic cell or tissue compared to a tumor cell.

As used herein, the term “positive for the expression of a marker” refers to the detection of expression of the marker by immunoassay, PCR or qPCR. Detection by immunoassay is preferred as it is capable of detecting individual expressing cells. Expression can be further distinguished as either “high” or “low” expression.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison. Polynucleotides having “substantial identity” to an endogenous sequence are typically share at least 80% identity with a recited nucleic acid sequence. In some aspects, lower identities are possible in view of the redundancy of the codons encoding polypeptides. In an aspect, the nucleic acids of the present application encode polypeptides having 90% or greater identity to the polypeptides of the present specification.

Sequence identity is typically measured using sequence analysis software that are widely available in the art. Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

As use herein, “NKT cell exhaustion” refers to loss of NKT cell function, which may occur as a result of an infection or a disease. NKT cell exhaustion is associated with increased expression of PD-1, TIM-3, and LAG-3, apoptosis, and reduced cytokine secretion. As used herein, NKT cells having “reduced exhaustion” are characterized by the ability to undergo multiple rounds of expansion. Generally, NKT cells having reduced exhaustion do not express the markers LAG-3, TIM-3 or PD-1, or express the markers at low levels. Further, examples of cells that retain NKT cell function and do not exhibit NKT cell exhaustion, include, but are not limited to CD62L(+) cells. As provided herein, cells that express exogenous transcriptional activators in the Wnt signaling pathway are cells that have reduced NKT cell exhaustion.

The terms “subject,” “individual,” and “patient,” are used interchangeably herein and refer to any vertebrate subject, including, without limitation, mammals, preferably a humans and other primates, including non-human primates such as laboratory animals including rodents such as mice, rats and guinea pigs; The term does not denote a particular age. Thus, both adult and newborn individuals are intended to be covered.

By “effective amount” is meant an amount sufficient to have a therapeutic effect. In one embodiment, an “effective amount” is an amount sufficient to arrest, ameliorate, or inhibit the continued proliferation, growth, or metastasis (e.g., invasion, or migration) of a neoplasia.

By a “heterologous nucleic acid molecule or polypeptide” is meant a nucleic acid molecule (e.g., acDNA, DNA or RNA molecule) or polypeptide that is not normally present in a cell or sample obtained from a cell. This nucleic acid may be from another organism, or it may be, for example, an mRNA molecule that is not normally expressed in a cell or sample.

By “immunoresponsive cell” is meant a cell that functions in an immune response or a progenitor, or progeny thereof.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

The term “obtaining” as in “obtaining the agent” is intended to include purchasing, synthesizing or otherwise acquiring the agent (or indicated substance or material).

By “modulate” is meant positively or negatively alter. Exemplary modulations include a 1%, 2%, 5%, 10%, 25%, 50%, 75%, or 100% change.

By “neoplasia” is meant a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasias can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasias include cancers, such as sarcomas, carcinomas, or plasmacytomas (malignant tumor of the plasma cells). Illustrative neoplasms for which the invention can be used include, but are not limited to leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

By “operably linked”, as used herein, is meant the linking of two or more biomolecules so that the biological functions, activities, and/or structure associated with the biomolecules are at least retained. In reference to polypeptides, the term means that the linking of two or more polypeptides results in a fusion polypeptide that retains at least some of the respective individual activities of each polypeptide component. The two or more polypeptides may be linked directly or via a linker. In reference to nucleic acids, the term means that a first polynucleotide is positioned adjacent to a second polynucleotide that directs transcription of the first polynucleotide when appropriate molecules (e.g., transcriptional activator proteins) are bound to the second polynucleotide.

As used herein, “populations of cells” refer to pluralities of cells and may further comprise mixtures of different cell types as well as homogenous populations. As used herein, a homogenous populations is

By “reduce” is meant to alter negatively by at least 5%. An alteration may be by 5%, 10%, 25%, 30%, 50%, 75%, or even by 100%.

By “recognize” is meant selectively binds a target. An NKT cell that recognizes a cell typically expresses a receptor that binds an antigen expressed by the cell.

By “reference” or “control” is meant a standard of comparison. For example, the immune response of a cell expressing lymphoid enhancer binding factor 1 (LEF1, Gene ID 51176), a CAR, or a combination thereof may be compared to the immune response of a corresponding non-engineered cell expressing CAR alone.

By “analog” is meant a structurally related polypeptide or nucleic acid molecule having the function of a reference polypeptide or nucleic acid molecule.

By “disease” is meant any condition or disorder that damages or interferes with the normal function of a cell, tissue, or organ. Examples of diseases include neoplasia or pathogen infection of cell.

As used herein, an “autonomous intra-ribosomal self-processing peptide” is a small peptide of 18 amino acids that avoids the need of proteinases to process a polyprotein into separate proteins. First discovered in foot-and-mouth disease virus, when introduced as a linker between two proteins, these peptides provides for the autonomous intra-ribosomal self-processing of polyproteins. Similar sequences have been identified in other members of the pircornaviradae. See de Felipe, “Skipping the co-expression problem: the new 2A ‘CHYSEL’ technology,” Genetic Vaccines and Therapy 2:13 (2004).

As used herein, “IRES” is refers to internal ribosomal entry sites (IRESs) that are sequences (˜0.5 kb) that allow the internal entry of ribosomes to translate the second cistron. As each cistron is translated from a different translational initiation event.

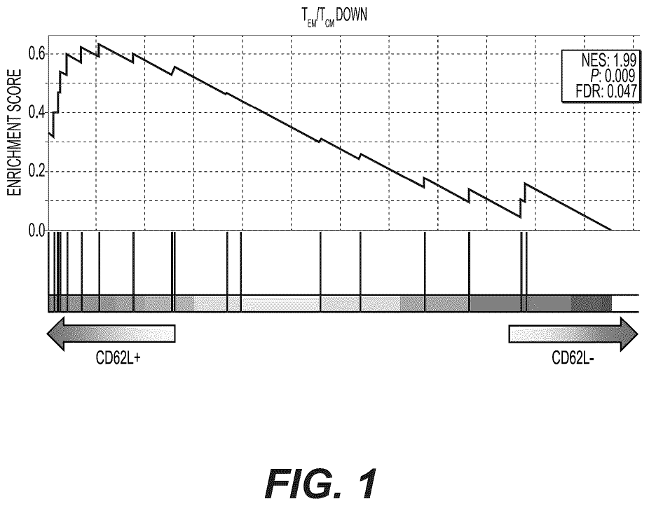

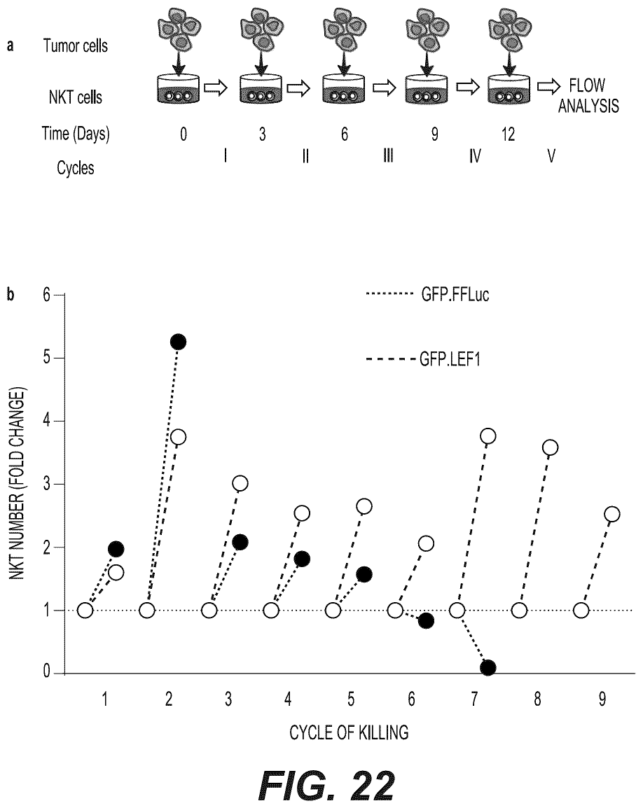

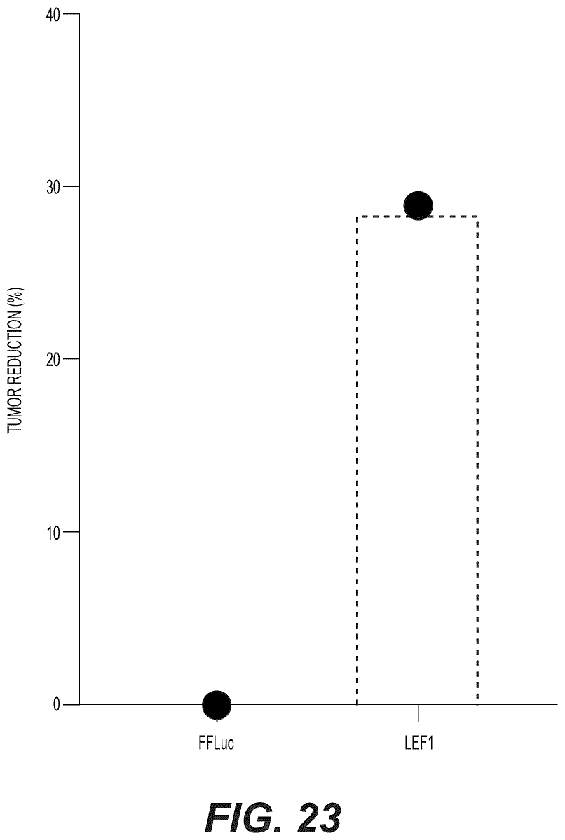

As used herein, the term “central memory NKT cell characteristic” refers to NKT cells having stem cell properties and that are capable of proliferating and retain cytotoxicity against antigen presenting cells. A representative sample of a central memory-like RNA expression profile is presented in FIG. 1 for CD62L+ iNKT cells. FIG. 1 shows enrichment for genes found in cells having a central memory CD4+ T cell signature (Abbas et al., 2009). Cells having a central memory NKT cell characteristic include, but are not necessarily limited to, CD62L+ iNKT cells. Populations of central memory NKT cells retain, after multiple rounds of challenge by antigen presenting cells, the ability to proliferate (FIG. 22) and cytotoxicity (FIG. 23). As shown herein, activation of the Wnt signaling pathway maintains the central memory NKT phenotype. In contrast, during culture NKT cells, including initial cultures CD62L+ iNKT cells lose the central memory characteristic and after repeated challenge lose the ability to proliferate and cytotoxicity.

As used herein, the term “engineering” refers to the genetic modification of a cell to introduce one or more exogenous nucleic acid sequences. Preferably, engineering introduced exogenous nucleic acid sequences that are transcribed and translated to express a protein. Introducing exogenous nucleic acid sequences can be performed using methods known in the art including transformation, transfection and transduction.

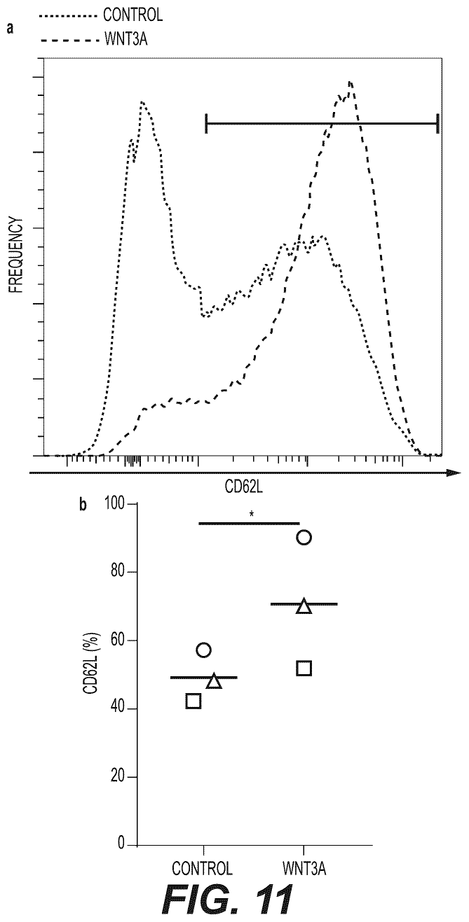

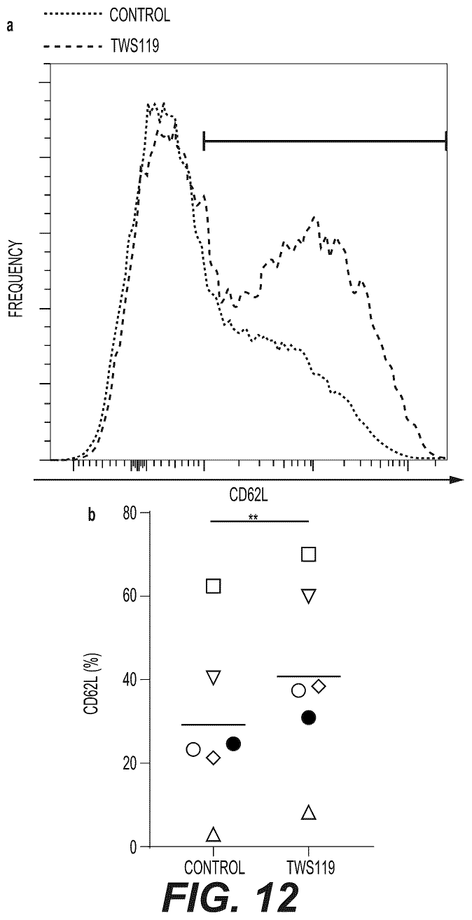

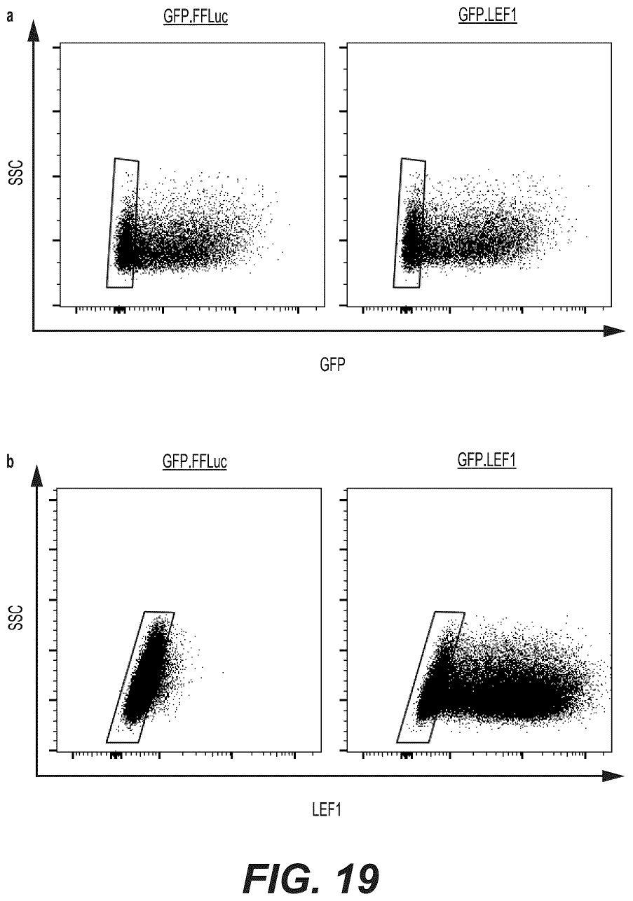

As used herein, the term “transcriptional activator in the Wnt signaling pathway” generally refers to proteins, that when exogenously expressed in a cell, activate genes downstream of Wnt/β-catenin signaling pathway. For example, adding the Wnt ligand as shown in FIG. 11 activates the Wnt pathway and increases the frequency of cells that are CD62L+, an example of NKT cells that have a central memory characteristic. A transcriptional activator in the Wnt signaling pathway includes the expression of positive regulators of Wnt signaling such as LEF1 (for example as shown in Examples 6 and 7 and FIGS. 19, 20, 27 and others) and inhibition of negative regulators, such as GSK30 (Example 3, FIG. 12. Also included in transcriptional activators of Wnt signaling are small molecule activators including, but not limited to those described in Blagodatski et al., “Small Molecule Wnt Pathway Modulators from Natural Sources: History, State of the Art and Perspectives,” Cells 9:589 (2020) and Verkaar et al., “Discovery of Novel Small Molecule Activators of 3-catenin Signaling,” PLoS ONE 6(4): e19185 (2011) and include inhibitors of negative regulators of Wnt signaling, such as TWS119 (Example 3, FIG. 12) (See Ding et al., Synthetic small molecules that control stem cell fate,” PNAS 100(13):7632-7 (2003)).

As used herein, the phrase “expresses a growth factor” refers to the exogenous expression of one or more growth factors, generally under the control of a heterologous promoter and more usually as part of a polyprotein downstream of a CHYSEL sequence.

The present specification provides for, and includes, genetically engineered NKT cells modified to express a transcriptional activator in the Wnt signaling pathway. In an aspect, the genetically engineered NKT cells further comprise an expression construct encoding a protein sequence CAR. In aspects, the genetically engineered NKT cell comprises two expression constructs a first encoding expression of a CAR and a second encoding for expression of a transcriptional activator in the Wnt signaling pathway. Also provided for are expression constructs that express a protein encoding for a transcriptional activator in the Wnt signaling pathway and up to three additional protein sequences. In aspects, the expression construct encodes a polyprotein comprising a CAR and a transcriptional activator in the Wnt signaling pathway. The expression construct encoding a polyprotein comprising a CAR and a transcriptional activator in the Wnt signaling pathway may further include additional coding sequences (e.g. cistrons). In aspects, the expression construct encoding a polyprotein comprises a CAR, a transcriptional activator in the Wnt signaling pathway, and a growth factor.

As provided herein, in aspects, a genetically engineered NKT cell is a Type I NKT cell. In an aspect the Type I NKT cell is a CD62L positive (CD62L+) NKT cell. Generally, the NKT cells of the present disclosure are isolated from human peripheral blood and have undergone less than 20 days of culture prior to introducing a gene construct to produce a genetically engineered NKT cell.

In aspects, the genetically engineered NKT cell of the present disclosure are further characterized by the expression of the cell markers CD4, CD28, 4-1BB, CD45RO (Gene ID5788), OX40, CCR7, and combinations thereof. The expression of these markers is closely associated with trafficking of the NKT cells to the tumor site where they can mediate anti-tumor responses. In further aspects, the genetically engineered NKT cells express markers of NKT cell survival and memory such as, but not limited to, S1PR1, IL-7Ra, IL21R. In aspects, the genetically engineered NKT cells of the present disclosure express low levels of the exhaustion markers TIM-3, LAG3, and PD-1.

In an aspect, the expression construct in a genetically engineered NKT cell comprises an expression construct that encodes a protein sequence for a transcriptional activator in the Wnt signaling pathway and up to three additional protein coding sequences. The additional protein coding sequences can each be expressed from their own promoter or in certain aspects expressed from a single promoter and expression of the additional coding sequences driven by an internal ribosomal entry sequence (IRES) for each coded protein.

In an aspect, the expression construct in a genetically engineered NKT cell encodes a protein sequence for a transcriptional activator in the Wnt signaling pathway and up to three additional protein coding sequences as part of a polyprotein with each protein coding sequence separated from the others by an autonomous intra-ribosomal self-processing peptide. In an aspect, the autonomous intra-ribosomal self-processing peptide is a foot-and-mouth disease virus (FMDV) 2A sequence or a related cis acting hydrolase element (CHYSEL).

The present specification provides for, and includes, genetically engineered NKT cells modified to express a transcriptional activator in the Wnt signaling pathway that includes, but is not limited to lymphoid enhancer binding factor 1 (LEF1, Gene ID 51176), beta-catenin ((CTNNB1, Gene ID 1499)), Smad3 (Gene ID 4088), HNF1 homeobox A (HNF1A, Gene ID: 6927 (alt. TCF1), transcription factor 7 (TCF7, Gene ID:6932 (alt. TCF1) and TLE family member 1, transcriptional corepressor (TLE 1, Gene ID 7088). In an aspect, the genetically engineered NKT cell comprises a expression construct encoding LEF1 is selected from the group consisting of Reference Sequence (RefSeq) ID NOs: NP_057353.1, NP_001124185.1, and NP_001124186.1. In an aspect, the genetically engineered NKT cell expresses LEF1 and a growth factor. In an aspect, the expression construct further encodes a growth factor selected from the group consisting of IL-15, IL-2, IL-4, IL-7, and combinations thereof.

Also included, and provided for, by the present disclosure is a genetically engineered NKT cell comprising an expression construct encoding a protein sequence for a transcriptional activator in the Wnt signaling pathway and encoding a protein sequence for a chimeric antigen receptor (CAR). In aspects, the genetically engineered NKT cell comprises separate expression constructs encoding the Wnt signaling pathway protein and a chimeric antigen receptor (CAR). In other aspects, the genetically engineered NKT cell comprises a single expression construct that encodes a Wnt signaling pathway protein and a chimeric antigen receptor (CAR). In further aspects, the Wnt signaling pathway protein and a chimeric antigen receptor (CAR) can be encoded as a single polyprotein separated by a CHYSEL sequence or as separate cistrons separated by IRES sequences. In a further aspect, that expression construct that encodes a Wnt signaling pathway protein and a chimeric antigen receptor (CAR) can further include a third protein encoding sequence for a growth factor selected from IL-15, IL-2, IL-4, or IL-7.

The present disclosure provides for and includes CAR proteins that comprise antibody recognition domains that recognize a cancer antigen. In aspects, the CAR comprises an antibody recognition domain for a cancer antigen, a spacer or hinge region, a transmembrane domain, and an endodomain. In an aspect, the antibody recognition domain is a single-chain variable fragment (scFv). In certain aspects the antibody recognition domain is directed at cancer antigens on the cell surface of cancer cells that express an antigen of interest, for example. In aspects, the endodomain includes a stimulatory domain, such as those derived from the T cell receptor z-chain. In other aspect, the stimulatory domains of the present specification include, but are not limited to, endodomains from co-stimulatory molecules such as CD27, CD28, 4-IBB, and OX40 or the signaling components of cytokine receptors such as IL7 and IL15. In aspects, co-stimulatory molecules are employed to enhance the activation, proliferation, and cytotoxicity of the NKT cells produced by the CAR after antigen engagement. In specific aspects, the co-stimulatory molecules are CD28, OX40, or 4-1BB.

In certain aspects, the genetically engineered NKT cells are modified to comprise at least a CAR in combination with expression of a transcriptional activator in the Wnt signaling pathway. In specific aspects, a particular NKT cell comprises expression of two or more CARs in combination with expression of a transcriptional activator in the Wnt signaling pathway. In an aspect, genetically engineered NKT cell comprises an expression construct for a transcriptional activator in the Wnt signaling pathway selected from the group consisting of lymphoid enhancer binding factor 1 (LEF1, Gene ID 51176), beta-catenin (CTNNB1), Smad3, and TLE 1. In and aspect, the transcriptional activator is LEF1 selected from the group consisting of Reference Sequence (RefSeq) ID NOs: NP_057353.1, NP_001124185.1, NP_001124186.1 in combination with one or more CARs.

Included, and provided by the present disclosure are cancer antigens such as Melanoma-associated antigen (MAGE), Preferentially expressed antigen of melanoma (PRAME), CD19, CD20, CD22, K-light chain, CD30, CD33, CD123, CD38, CD138, RORI, ErbB2, ErbB3/4, EGFr vIII, carcinoembryonic antigen, EGP2, EGP40, HER2, mesothelin, TAG72, PSMA, NKG2D ligands, B7-H6, IL-13 receptor a2, MUC1, MUC16, CA9, GD2, GD3, HMW-MAA, CD171, Lewis Y, G250/CAIX, HLA-AI MAGE Al, HLA-A2 NY-ESO-1, PSC1, folate receptor-a, CD44v6, CD44v7/8, 8H9, NCAM, VEGF receptors, 5T4, Fetal AchR, or CD44v6. In an aspect, the cancer antigen is selected from the group consisting of CD19, GD2, and GPC3. In another aspect, the cancer antigen is CD19. In an aspect, the cancer antigen is GD2. In yet another aspect, the cancer antigen is GPC3.

Also included and provided for by the present disclosure are genetically engineered NKT cell cells comprising two or more CAR molecules that recognize cancer antigens selected from the group consisting of MAGE, PRAME, CD19, CD20, CD22, K-light chain, CD30, CD33, CD123, CD38, CD138, RORI, ErbB2, ErbB3/4, EGFr vIII, carcinoembryonic antigen, EGP2, EGP40, HER2, mesothelin, TAG72, PSMA, NKG2D ligands, B7-H6, IL-13 receptor a2, MUC1, MUC16, CA9, GD2, GD3, HMW-MAA, CD171, Lewis Y, G250/CAIX, HLA-AI MAGE Al, HLA-A2 NY-ESO-1, PSC1, folate receptor-a, CD44v6, CD44v7/8, 8H9, NCAM, VEGF receptors, 5T4, Fetal AchR, and CD44v6. Genetically engineered NKT cells having two or more CAR molecules further include a construct for expressing a Wnt signaling pathway protein and may further include recombinant sequences for expressing a growth factor selected from IL-2, IL-4, IL-7, IL-15, or IL-21. Expression constructs can be prepared separately, or more conveniently as one or more polyprotein expression constructs with CHYSEL sequences.

In general, an ectodomain of the CAR encompasses a signal peptide, antigen recognition domain, and a spacer that links the antigen recognition domain to the transmembrane domain. The antigen recognition domain generally will comprise a single chain variable fragment (scFv) specific for a particular cancer antigen. However, in cases wherein there are two or more CARs in the same cell, the second CAR may comprise an scFv specific for another particular antigen. Examples of cancer antigens include the antigens described above.

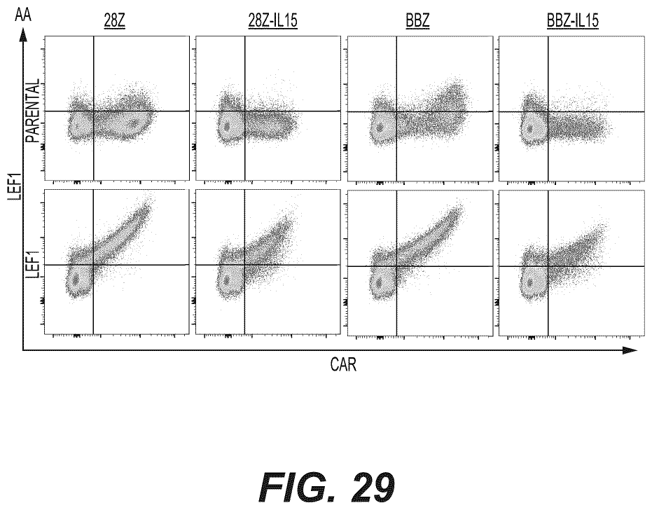

Specific examples of polyprotein encoding expression constructs are presented, but not limited to, those in FIG. 29a-1. As shown, the expression constructs include antibody recognition domains that recognize the cancer antigens, CD19, GD2, or GPC3, in combination with the Wnt transcription factor LEF1. Also shown are expression constructs that further encode the growth factor IL-15.

Examples of hinge regions for the ectodomain include the CH2CH3 region of immunoglobulin, the hinge region from IgG1, and portions of CD3. The transmembrane region may be of any kind, although in some cases it is CD28.

In general, the endodomain of the CAR of the disclosure is utilized for signal transmission in the cell after antigen recognition and cluster of the receptors. The most commonly used endodomain component is CD3-zeta that contains 3 ITAMs and that transmits an activation signal to the T cell after the antigen is bound. In some embodiments, additional co-stimulatory signaling is utilized, such as CD3-zeta in combination with CD28, 4-IBB, and/or OX40.

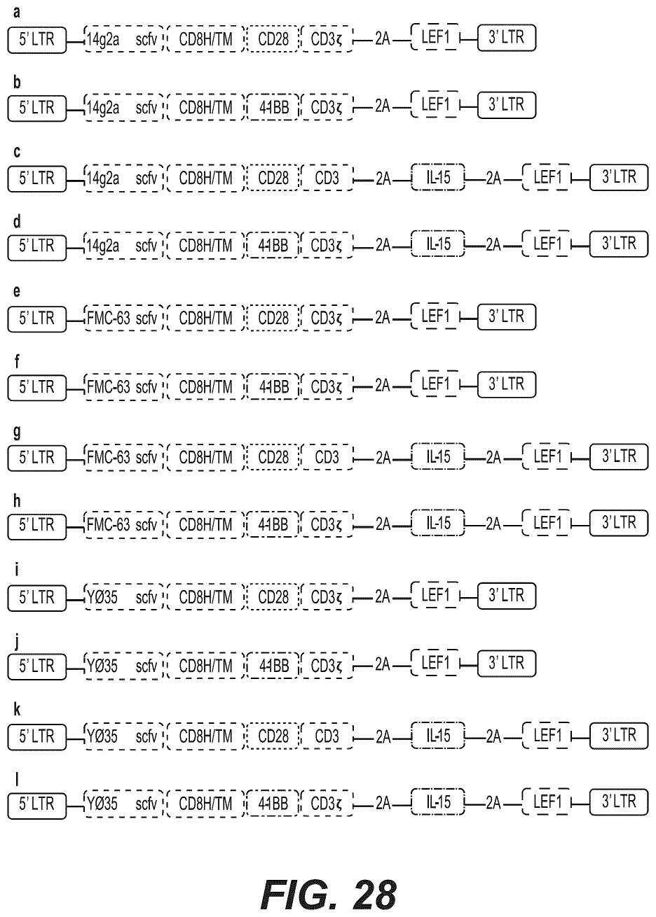

While the expression constructs are shown for expressing each of the proteins in a particular order, the present disclosure is not so limited. For example, the construct of FIG. 28c can be configured to express IL-15, linked to CAR, and linked to LEF1, or configured to express LEF1, linked to IL-15, linked to CAR. Further, it would be evident to a person of skill in the art that the scFV sequences (CD19-specific antibody FMC-63, the GD2-specific antibody 14G2a, or the GD3 specific antibody Y035) can be substituted with antibody sequences of the cancer antigens identified above. Similarly, the LEF1 sequence can be substituted with the Wnt signaling pathway proteins beta-catenin (CTNNB1), Smad3, or TLE 1 and in a similar manner, the IL-15 sequences can be replaced with any one of IL-2, IL-4, IL-7, or IL-21. As provided herein, the genetically engineered NKT cells expressing a Wnt signaling pathway protein prevent exhaustion of the NKT cell during culture and provide for persistent expansion potential after multiple rounds of challenge, for example with CD1+ J32 leukemia cells.

The present disclosure provides for, and includes, populations of genetically engineered NKT cell cells. In some aspects, the genetically engineered NKT cells can be a purified population of genetically engineered NKT cells. Generally, NKT cells are present in, and obtainable from PBMCs though at low levels. NKT cells can be isolated by positive or negative selection techniques, expanded, and transfected or transduced with the vectors of the present disclosure to prepare genetically engineered NKT cells. The populations of genetically engineered NKT cells of the present disclosure can comprise any number of cells but generally are populations of at least 100,000 cells or more. In aspects, the population can be between 10⁵ to 10¹⁰ cells. Included an provided for are mixed populations wherein the genetically engineered NKT cells are a subpopulation.

In other aspects, the genetically engineered NKT cells are a subpopulation of cells. NKT cells, or alternatively the NKT cells can be in a population with cells of a different type, such as B cells and/or other peripheral blood cells. The NKT cells can be a purified population of a subset of T cells, such as CD4+ T cells, or they can be a population of NKT cells within a subset of T cells. In another embodiment of the invention, the T cells are T cell clones that have been maintained in culture for extended periods of time. T cell clones can be transfected or transduced to different degrees. In a specific embodiment, the T cells are a T cell clone that proliferates indefinitely in culture.

The present disclosure provides for, and includes a population of cells comprising a plurality of genetically engineered NKT cells comprising expression construct encoding a protein sequence for a transcriptional activator in the Wnt signaling pathway. In an aspect, the population of genetically engineered NKT cells are a mixed population wherein the population further comprises including non-engineered Type I and Type II NKT cells, irradiated PBMC cells, non-NKT cells, or other non-engineered cells. In aspects the population of genetically engineered NKT cells comprise greater than 10% of the total cell population. In another aspect, the proportion of genetically engineered NKT cells is 25 to 30% of the total population of cells.

In certain aspects, the plurality of genetically engineered NKT cells according to the present disclosure comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the total cell population. In certain aspects, the plurality of genetically engineered NKT cells according to the present disclosure comprises about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 10% to about 80%, about 10% to about 85%, about 10% to about 90%, about 10% to about 95%, about 10% to 100%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 15% to about 80%, about 15% to about 85%, about 15% to about 90%, about 15% to about 95%, about 15% to 100%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 20% to about 80%, about 20% to about 85%, about 20% to about 90%, about 20% to about 95%, about 20% to 100%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 25% to about 80%, about 25% to about 85%, about 25% to about 90%, about 25% to about 95%, about 25% to 100%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 30% to about 80%, about 30% to about 85%, about 30% to about 90%, about 30% to about 95%, about 30% to 100%, about 35% to about 40%, about 35% to about 45%, about 35% to about 50%, about 35% to about 55%, about 35% to about 60%, about 35% to about 65%, about 35% to about 70%, about 35% to about 75%, about 35% to about 80%, about 35% to about 85%, about 35% to about 90%, about 35% to about 95%, about 35% to 100%, about 40% to about 45%, about 40% to about 50%, about 40% to about 55%, about 40% to about 60%, about 40% to about 65%, about 40% to about 70%, about 40% to about 75%, about 40% to about 80%, about 40% to about 85%, about 40% to about 90%, about 40% to about 95%, about 40% to 100%, about 45% to about 50%, about 45% to about 55%, about 45% to about 60%, about 45% to about 65%, about 45% to about 70%, about 45% to about 75%, about 45% to about 80%, about 45% to about 85%, about 45% to about 90%, about 45% to about 95%, about 45% to 100%, about 50% to about 55%, about 50% to about 60%, about 50% to about 65%, about 50% to about 70%, about 50% to about 75%, about 50% to about 80%, about 50% to about 85%, about 50% to about 90%, about 50% to about 95%, about 50% to 100%, about 55% to about 60%, about 55% to about 65%, about 55% to about 70%, about 55% to about 75%, about 55% to about 80%, about 55% to about 85%, about 55% to about 90%, about 55% to about 95%, about 55% to 100%, about 60% to about 65%, about 60% to about 70%, about 60% to about 75%, about 60% to about 80%, about 60% to about 85%, about 60% to about 90%, about 60% to about 95%, about 60% to 100%, about 65% to about 70%, about 65% to about 75%, about 65% to about 80%, about 65% to about 85%, about 65% to about 90%, about 65% to about 95%, about 65% to 100%, about 70% to about 75%, about 70% to about 80%, about 70% to about 85%, about 70% to about 90%, about 70% to about 95%, about 70% to 100%, about 75% to about 80%, about 75% to about 85%, about 75% to about 90%, about 75% to about 95%, about 75% to 100%, about 80% to about 85%, about 80% to about 90%, about 80% to about 95%, about 80% to 100%, about 85% to about 90%, about 85% to about 95%, about 85% to 100%, about 90% to about 95%, about 90% to 100%, or about 95% to 100% of the total cell population. In certain aspects, the plurality of genetically engineered NKT cells according to the present disclosure comprises about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or 100% of the total cell population.

The present disclosure provides for, and includes, genetically engineered NKT cells wherein said plurality of genetically engineered NKT cells comprises at least 50% CD62L(+) NKT cells.

In certain aspects, the plurality of genetically engineered NKT cells according to the present disclosure comprises at least 50%, at least 55%, at least 60%, at least 65%, at least 70%, at least 75%, at least 80%, at least 85%, at least 90%, or at least 95% CD62L(+) NKT cells. In certain aspects, the plurality of genetically engineered NKT cells according to the present disclosure comprises about 50% to about 55%, about 50% to about 60%, about 50% to about 65%, about 50% to about 70%, about 50% to about 75%, about 50% to about 80%, about 50% to about 85%, about 50% to about 90%, about 50% to about 95%, about 50% to 100%, about 55% to about 60%, about 55% to about 65%, about 55% to about 70%, about 55% to about 75%, about 55% to about 80%, about 55% to about 85%, about 55% to about 90%, about 55% to about 95%, about 55% to 100%, about 60% to about 65%, about 60% to about 70%, about 60% to about 75%, about 60% to about 80%, about 60% to about 85%, about 60% to about 90%, about 60% to about 95%, about 60% to 100%, about 65% to about 70%, about 65% to about 75%, about 65% to about 80%, about 65% to about 85%, about 65% to about 90%, about 65% to about 95%, about 65% to 100%, about 70% to about 75%, about 70% to about 80%, about 70% to about 85%, about 70% to about 90%, about 70% to about 95%, about 70% to 100%, about 75% to about 80%, about 75% to about 85%, about 75% to about 90%, about 75% to about 95%, about 75% to 100%, about 80% to about 85%, about 80% to about 90%, about 80% to about 95%, about 80% to 100%, about 85% to about 90%, about 85% to about 95%, about 85% to 100%, about 90% to about 95%, about 90% to 100%, or about 95% to 100% CD62L(+) NKT cells.

In certain aspects, the plurality of genetically engineered NKT cells according to the present disclosure comprises about 50%, about 55%, about 60%, about 65%, about 70%, about 75%, about 80%, about 85%, about 90%, about 95%, or 100% CD62L(+) NKT cells.

The present disclosure provides for, and includes, genetically engineered NKT cells wherein said plurality of genetically engineered NKT cells express LEF1. In an aspect, at least percent of a plurality of genetically engineered NKT cells express LEF1.

In certain aspects, at least 30%, at least 35%, at least 40%, at least 45%, at least 50%, at least 55%, at least 60%, at least 65%, at least 70%, at least 75%, at least 80%, at least 85%, at least 90%, or at least 95% of the plurality of genetically engineered NKT cells according to the present disclosure express LEF1. In certain aspects, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 30% to about 80%, about 30% to about 85%, about 30% to about 90%, about 30% to about 95%, about 30% to 100%, about 35% to about 40%, about 35% to about 45%, about 35% to about 50%, about 35% to about 55%, about 35% to about 60%, about 35% to about 65%, about 35% to about 70%, about 35% to about 75%, about 35% to about 80%, about 35% to about 85%, about 35% to about 90%, about 35% to about 95%, about 35% to 100%, about 40% to about 45%, about 40% to about 50%, about 40% to about 55%, about 40% to about 60%, about 40% to about 65%, about 40% to about 70%, about 40% to about 75%, about 40% to about 80%, about 40% to about 85%, about 40% to about 90%, about 40% to about 95%, about 40% to 100%, about 45% to about 50%, about 45% to about 55%, about 45% to about 60%, about 45% to about 65%, about 45% to about 70%, about 45% to about 75%, about 45% to about 80%, about 45% to about 85%, about 45% to about 90%, about 45% to about 95%, about 45% to 100%, about 50% to about 55%, about 50% to about 60%, about 50% to about 65%, about 50% to about 70%, about 50% to about 75%, about 50% to about 80%, about 50% to about 85%, about 50% to about 90%, about 50% to about 95%, about 50% to 100%, about 55% to about 60%, about 55% to about 65%, about 55% to about 70%, about 55% to about 75%, about 55% to about 80%, about 55% to about 85%, about 55% to about 90%, about 55% to about 95%, about 55% to 100%, about 60% to about 65%, about 60% to about 70%, about 60% to about 75%, about 60% to about 80%, about 60% to about 85%, about 60% to about 90%, about 60% to about 95%, about 60% to 100%, about 65% to about 70%, about 65% to about 75%, about 65% to about 80%, about 65% to about 85%, about 65% to about 90%, about 65% to about 95%, about 65% to 100%, about 70% to about 75%, about 70% to about 80%, about 70% to about 85%, about 70% to about 90%, about 70% to about 95%, about 70% to 100%, about 75% to about 80%, about 75% to about 85%, about 75% to about 90%, about 75% to about 95%, about 75% to 100%, about 80% to about 85%, about 80% to about 90%, about 80% to about 95%, about 80% to 100%, about 85% to about 90%, about 85% to about 95%, about 85% to 100%, about 90% to about 95%, about 90% to 100%, or about 95% to 100% of the plurality of genetically engineered NKT cells according to the present disclosure express LEF1. In certain aspects, about 30%, about 35%, about 40%, about 45%, about 50%, about 55%, about 60%, about 65%, about 70%, about 75%, about 80%, about 85%, about 90%, about 95%, or 100% of the plurality of genetically engineered NKT cells according to the present disclosure express LEF1.

The present disclosure also provides for a therapeutically effective amount of the population of cells as described herewith.

The present disclosure provides for a chimeric antigen receptor expression construct comprising (a) a chimeric antigen receptor (CAR) coding sequence comprising an ectodomain sequence, a transmembrane domain sequence, and an endodomain sequence, and (b) a protein coding sequence for a transcriptional activator in the Wnt signaling pathway.

In one aspect, the expression constructs or chimeric antigen receptor expression constructs according to the present disclosure further comprises a protein coding sequence for an interleukin (IL). In certain aspects, the protein coding sequence encodes an IL-2, an IL-4, and IL-7, an IL-12, an IL-15, an IL-21, or a combination thereof. In certain aspects, the protein coding sequence encodes an IL-2, an IL-4, and IL-7, an IL-15, or a combination thereof. In one aspect, the protein coding sequence encodes an IL-2. In one aspect, the protein coding sequence encodes an IL-4. In one aspect, the protein coding sequence encodes an IL-7. In one aspect, the protein coding sequence encodes an IL-15. In one aspect, the interleukin sequence is from human. In another aspect, the interleukin sequence is exogenous. In one particular aspect, the expression construct comprises a protein coding sequence for a human IL-15.

In one aspect, the expression constructs or chimeric antigen receptor expression constructs according to the present disclosure further comprises an inducible suicide gene. Non-limiting examples of inducible suicide genes include an inducible caspase-9 suicide gene and a thymidine kinase (sr39 TK). In one aspect, the inducible caspase-9 suicide gene in the expression construct is activated by AP20187, API 903, or a mixture thereof. In another aspect, the In one aspect, the thymidine kinase in the expression construct is activated by ganciclovir. In one aspect, methods according to the present disclosure may comprise administering AP20187, API 903, or a mixture thereof to the subject to activate the inducible caspase-9 suicide gene. In another aspect, methods according to the present disclosure may comprise administering ganciclovir to the subject to activate the thymidine kinase.

In one aspect, the expression constructs or chimeric antigen receptor expression constructs according to the present disclosure further comprises a protein coding sequence for a CD34 tag.

In one aspect, the expression constructs or chimeric antigen receptor expression constructs according to the present disclosure further comprises protein coding sequences for additional components.

In one aspect, the expression constructs according to the present disclosure comprises one or more linkers between the protein coding sequences to allow autonomous intra-ribosomal self-processing of polyproteins. Non-limiting examples of the linker sequences include the “2A” sequences peptides derived from foot-and-mouth disease virus (FMDV), and a related cis-acting hydrolase element (CHYSEL). In one aspect, the expression construct comprises a first linker sequence between the CAR coding sequence and the protein coding sequence for the transcriptional activator in the Wnt signaling pathway. In another aspect, the expression construct comprises a second, a third, a fourth, or more linker sequences separating the protein coding sequence an interleukin, the inducible suicide gene, or other components of the expression constructs.

In one aspect, the CAR coding sequence and the protein coding sequence for a transcriptional activator in the Wnt signaling pathway are in frame in the expression constructs according to the present disclosure. In another aspect, the CAR coding sequence, the protein coding sequence for a transcriptional activator in the Wnt signaling pathway, and the protein coding sequence for an interleukin are in frame. In a further aspect, the CAR coding sequence, the protein coding sequence for a transcriptional activator in the Wnt signaling pathway, the protein coding sequence for an interleukin, and one or more additional protein coding sequences are in frame.

In one aspect, the transcriptional activator in the Wnt signaling pathway in the expression constructs according to the present disclosure is lymphoid enhancer binding factor 1 (LEF1). In another aspect, the transcriptional activator is beta-catenin (CTNNB1), Smad3, or TLE 1. In one aspect, the LEF1 sequence is from human.

In one aspect, the ectodomain sequence in the expression constructs according to the present disclosure comprises an antigen recognition domain and a spacer/hinge domain that links the antigen recognition domain to the transmembrane domain. In another aspect, the ectodomain sequence further comprises a signal peptide. Non-limiting examples of hinge regions for the ectodomain include the CH2CH3 region of immunoglobulin, the hinge region from IgG1, and portions of CD3. In one aspect, the hinge region of the ectodomain comprises CH8H.

In certain aspect, the antigen recognition domain comprises a single-chain variable fragment (scFv). In certain aspect, the antigen recognition domain recognizes a cancer antigen on the cell surface of cancel cells. Non-limiting examples of cancer antigens include any one of Melanoma-associated antigen (MAGE), Preferentially expressed antigen of melanoma (PRAME), CD19, CD20, CD22, K-light chain, CD30, CD33, CD123, CD38, CD138, ROR1, ErbB2, ErbB3/4, EGFr vIII, carcinoembryonic antigen, EGP2, EGP40, HER2, mesothelin, TAG72, PSMA, NKG2D ligands, B7-H6, IL-13 receptor a2, MUC1, MUC16, CA9, GD2, GD3, HMW-MAA, CD171, Lewis Y, G250/CAIX, HLA-AI MAGE Al, HLA-A2 NY-ESO-1, PSC1, folate receptor-a, CD44v6, CD44v7/8, 8H9, NCAM, VEGF receptors, 5T4, Fetal AchR, NKG2D ligands, or CD44v6. In some cases, the antigen recognition domain recognizes CD19, CD22, CD30, GD2, GPC3, CSPG4, HER2, CEA, or Mesothelin. In one particular aspect, the antigen recognition domain comprises a single-chain variable fragment (scFv) from the CD19-specific antibody FMC-63. In another particular aspect, the antigen recognition domain comprises a single-chain variable fragment (scFv) from the GD2-specific antibody 14G2a.

In one aspect, the endodomain sequence in the expression construct according to the present disclosure comprises a cytoplasmic signaling domain, such as those derived from the T cell receptor ζ-chain, in order to produce stimulatory signals for NKT cell proliferation and effector function following engagement of the antigen recognition domain with the target antigen. Non-limiting examples of the endodomain sequences include endodomains from co-stimulatory molecules such as CD27, CD28, 4-IBB, and OX40 or the signaling components of cytokine receptors such as IL7 and IL15. In certain aspects, co-stimulatory molecules are employed to enhance the activation, proliferation, and cytotoxicity of the NKT cells after antigen engagement. In specific aspects, the co-stimulatory molecules are CD28, OX40, and 4-1BB. In one aspect, the endodomain of the CAR according to the present disclosure is utilized for signal transmission in the cell after antigen recognition and cluster of the receptors. In one aspect, the endodomain comprises a CD3-zeta that contains 3 ITAMs and that transmits an activation signal to the NKT cell after the antigen is bound. In certain aspects, additional co-stimulatory signaling is utilized, such as CD3-zeta in combination with CD28, 4-IBB, and/or OX40. In one particular aspect, the endodomain sequence comprises the signal sequence of 4-1BB fused in-frame to a CD3-zeta chain.

The transmembrane domain may be of any kind. In one aspect, the transmembrane domain comprises the transmembrane domain of CD28. In another aspect, the transmembrane domain comprises the transmembrane domain of CD8.

In one particular aspect, the CAR.CD19 and CAR.GD2 constructs are made as previously described (Heczey et al., 2014: Pule et al., A chimeric T cell antigen receptor that augments cytokine release and supports clonal expansion of primary human T cells. Mol. Ther. 2005; 12(5):933-941) and contain a scFv from the CD19-specific antibody FMC-63 or the GD2-specific antibody 14G2a connected via a short spacer derived from the IgG1 hinge region to the transmembrane domain derived from CD8a, followed by signaling endodomain sequences of 4-IBB fused with (chain.

Expression constructs according to the present disclosure can be introduced into the cells as one or more DNA molecules or constructs, where there may be at least one marker that will allow for selection of host cells that contain the construct(s). The constructs can be prepared in conventional ways, where the genes and regulatory regions may be isolated, as appropriate, ligated, cloned in an appropriate cloning host, analyzed by restriction or sequencing, or other convenient means. The constructs once completed and demonstrated to have the appropriate sequences may then be introduced into the CTL by any convenient means. The constructs may be integrated and packaged into non-replicating, defective viral genomes like Adenovirus, Adeno-associated virus (AAV), or Herpes simplex virus (HSV) or others, including retroviral vectors, for infection or transduction into cells. The constructs may include viral sequences for transfection, if desired. Alternatively, the construct may be introduced by fusion, electroporation, biolistics, transfection, lipofection, or the like. The host cells may be grown and expanded in culture before introduction of the construct(s), followed by the appropriate treatment for introduction of the construct(s) and integration of the construct(s). The cells are then expanded and screened by virtue of a marker present in the construct. Various markers that may be used successfully include hprt, neomycin resistance, thymidine kinase, hygromycin resistance, etc.

The present disclosure also provides for genetically engineered NKT cells comprising the chimeric antigen receptor expression constructs as disclosure herewith.

The present disclosure provides for, and includes, methods for maintaining NKT cell expansion potential comprising the steps of engineering NKT cells to express at least a protein coding sequence comprising a transcriptional activator in the Wnt signaling pathway and culturing the engineered NKT cells to prepare a population of genetically engineered NKT cells with persistent expansion potential. In an aspect, the method further provides for isolating PBMCs from a donor, separating NKT cells from the PBMCs, and expanding the separated NKT cells in the presence of at least aGalCer, IL-2, and IL-21 for at least 1 day to prepare NKT cells for genetic engineering. The cells are then transfected or transduced with the expression vectors described herein and cultured to prepare NKT cells with preserved expansion potential.

In aspects, the methods provide for expression of a transcriptional activator in the Wnt signaling pathway that includes, but is not limited to lymphoid enhancer binding factor 1 (LEF1, Gene ID 51176), beta-catenin ((CTNNB1, Gene ID 1499)), Smad3 (Gene ID 4088), and TLE 1 (Gene ID 7088). In an aspect, the genetically engineered NKT cell comprises a expression construct encoding LEF1 is selected from the group consisting of Reference Sequence (RefSeq) ID NOs: NP_057353.1, NP_001124185.1, and NP_001124186.1. In an aspect, the genetically engineered NKT cell expresses LEF1 and a growth factor. In an aspect, the expression construct further encodes a growth factor selected from the group consisting of IL-15, IL-2, IL-4, IL-7, and combinations thereof.

In certain aspects, the NKT cells separated by the methods according to the present disclosure are expanded in the presence of suitable stimulants (e.g., αGalCer, IL-2, and IL-21) for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, or at least 20 days to prepare the NKT cells for genetic engineering. In certain aspects, the NKT cells separated by the methods according to the present disclosure are expanded in the presence of suitable stimulants (e.g., αGalCer, IL-2, and IL-21) for about 1 to 2 days, about 1 to 3 days, about 1 to 4 days, about 1 to 5 days, about 1 to 6 days, about 1 to 7 days, about 1 to 8 days, about 1 to 9 days, about 1 to 10 days, about 1 to 11 days, about 1 to 12 days, about 1 to 13 days, about 1 to 14 days, about 1 to 15 days, about 1 to 16 days, about 1 to 17 days, about 1 to 18 days, about 1 to 19 days, about 1 to 20 days, about 2 to 3 days, about 2 to 4 days, about 2 to 5 days, about 2 to 6 days, about 2 to 7 days, about 2 to 8 days, about 2 to 9 days, about 2 to 10 days, about 2 to 11 days, about 2 to 12 days, about 2 to 13 days, about 2 to 14 days, about 2 to 15 days, about 2 to 16 days, about 2 to 17 days, about 2 to 18 days, about 2 to 19 days, about 2 to 20 days, about 3 to 4 days, about 3 to 5 days, about 3 to 6 days, about 3 to 7 days, about 3 to 8 days, about 3 to 9 days, about 3 to 10 days, about 3 to 11 days, about 3 to 12 days, about 3 to 13 days, about 3 to 14 days, about 3 to 15 days, about 3 to 16 days, about 3 to 17 days, about 3 to 18 days, about 3 to 19 days, about 3 to 20 days, about 4 to 5 days, about 4 to 6, about 4 to 7, about 4 to 8, about 4 to 9, about 4 to 10, about 4 to 11, about 4 to 12, about 4 to 13 days, about 4 to 14 days, about 4 to 15 days, about 4 to 16 days, about 4 to 17 days, about 4 to 18 days, about 4 to 19 days, about 4 to 20 days, about 5 to 6 days, about 5 to 7 days, about 5 to 8 days, about 5 to 9 days, about 5 to 10 days, about 5 to 11 days, about 5 to 12 days, about 5 to 13 days, about 5 to 14 days, about 5 to 15 days, about 5 to 16 days, about 5 to 17 days, about to 18 days, about 5 to 19 days, about 5 to 20 days, about 6 to 7 days, about 6 to 8 days, about 6 to 9 days, about 6 to 10 days, about 6 to 11 days, about 6 to 12 days, about 6 to 13 days, about 6 to 14 days, about 6 to 15 days, about 6 to 16 days, about 6 to 17 days, about 6 to 18 days, about 6 to 19 days, about 6 to 20 days, about 7 to 8 days, about 7 to 9 days, about 7 to 10 days, about 7 to 11 days, about 7 to 12 days, about 7 to 13, about 7 to 14, about 7 to 15, about 7 to 16, about 7 to 17, about 7 to 18, about 7 to 19 days, about 7 to 20 days, about 8 to 9 days, about 8 to 10 days, about 8 to 11 days, about 8 to 12 days, about 8 to 13 days, about 8 to 14 days, about 8 to 15, days about 8 to 16 days, about 8 to 17 days, about 8 to 18 days, about 8 to 19 days, about 8 to 20 days, about 9 to 10 days, about 9 to 11 days, about 9 to 12 days, about 9 to 13 days, about 9 to 14 days, about 9 to 15, days about 9 to 16 days, about 9 to 17 days, about 9 to 18 days, about 9 to 19 days, about 9 to 20 days, about 10 to 11 days, about 10 to 12 days, about 10 to 13 days, about 10 to 14 days, about 10 to 15 days, about 10 to 16 days, about 10 to 17 days, about to 18 days, about 10 to 19 days, about 10 to 20 days, about 11 to 12 days, about 11 to 13 days, about 11 to 14 days, about 11 to 15 days, about 11 to 16 days, about 11 to 17 days, about 11 to 18 days, about 11 to 19 days, about 11 to 20 days, about 12 to 13 days, about 12 to 14 days, about 12 to 15 days, about 12 to 16 days, about 12 to 17 days, about 12 to 18 days, about 12 to 19 days, about 12 to 20 days, about 13 to 14 days, about 13 to 15 days, about 13 to 16 days, about 13 to 17 days, about 13 to 18 days, about 13 to 19 days, about 13 to 20 days, about 14 to 15 days, about 14 to 16 days, about 14 to 17 days, about 14 to 18 days, about 14 to 19 days, about 14 to 20 days, about 15 to 16 days, about 15 to 17 days, about 15 to 18 days, about to 19 days, about 15 to 20 days, about 16 to 17 days, about 16 to 18 days, about 16 to 19 days, about 16 to 20 days, about 17 to 18 days, about 17 to 19 days, about 17 to 20 days, about 18 to 19 days, about 18 to 20 days, or about 19 to 20 days to prepare the NKT cells for genetic engineering. In certain aspects, the NKT cells separated by the methods according to the present disclosure are expanded in the presence of suitable stimulants (e.g., aGalCer, IL-2, and IL-21) for about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, or about 20 days to prepare the NKT cells for genetic engineering.

Also included, and provided, by the present disclosure are methods of maintaining NKT cell expansion potential comprising transfecting or transducing NKT cells with an expression construct comprising (a) a chimeric antigen receptor (CAR) coding sequence comprising an ectodomain sequence, a transmembrane domain sequence, and an endodomain sequence and (b) a sequence encoding a protein sequence for a transcriptional activator in the Wnt signaling pathway to prepare a genetically engineered NKT cell.

In aspects, the methods provide for expression of a transcriptional activator in the Wnt signaling pathway that includes, but is not limited to lymphoid enhancer binding factor 1 (LEF1, Gene ID 51176), beta-catenin ((CTNNB1, Gene ID 1499)), Smad3 (Gene ID 4088), and TLE 1 (Gene ID 7088). In an aspect, the genetically engineered NKT cell comprises a expression construct encoding LEF1 is selected from the group consisting of Reference Sequence (RefSeq) ID NOs: NP_057353.1, NP_001124185.1, and NP_001124186.1. In an aspect, the genetically engineered NKT cell expresses LEF1 and a growth factor. In an aspect, the expression construct further encodes a growth factor selected from the group consisting of IL-15, IL-2, IL-4, IL-7, and combinations thereof.

In certain aspects, the methods of maintaining NKT cell expansion potential includes genetically engineering NKT cells to comprise at least a CAR in combination with expression of a transcriptional activator in the Wnt signaling pathway. In specific aspects, NKT cells are engineered to express two or more CARs in combination with expression of a transcriptional activator in the Wnt signaling pathway. In an aspect, genetically engineering the NKT cell comprises introducing an expression construct for a transcriptional activator in the Wnt signaling pathway selected from the group consisting of lymphoid enhancer binding factor 1 (LEF1, Gene ID 51176), beta-catenin (CTNNB1), Smad3, and TLE 1. In and aspect, the transcriptional activator is LEF1 selected from the group consisting of Reference Sequence (RefSeq) ID NOs: NP_057353.1, NP_001124185.1, NP_001124186.1 in combination with one or more CARs.

Included, and provided by the methods of maintaining NKT cell expansion potential of are expression of CARs recognizing cancer antigens such as Melanoma-associated antigen (MAGE), Preferentially expressed antigen of melanoma (PRAME), CD19, CD20, CD22, K-light chain, CD30, CD33, CD123, CD38, CD138, RORl, ErbB2, ErbB3/4, EGFr vIII, carcinoembryonic antigen, EGP2, EGP40, HER2, mesothelin, TAG72, PSMA, NKG2D ligands, B7-H6, IL-13 receptor a2, MUC1, MUC16, CA9, GD2, GD3, HMW-MAA, CD171, Lewis Y, G250/CAIX, HLA-AI MAGE Al, HLA-A2 NY-ESO-1, PSC1, folate receptor-a, CD44v6, CD44v7/8, 8H9, NCAM, VEGF receptors, 5T4, Fetal AchR, or CD44v6. In an aspect, the cancer antigens introduced to genetically engineer NKT cells are selected from the group consisting of CD19, GD2, and GPC3. In another aspect, the antibody recognition domain of the CAR recognized the cancer antigen CD19. In an aspect, the cancer antigen recognized by the antibody recognition domain is GD2. In yet another aspect, the cancer antigen recognized by the antibody recognition domain GPC3. Methods to introduce expression constructs are known in the art. In aspects or the present disclosure the genetic engineering includes and provides for introducing the constructs of the present disclosure using retrovirus or lentivirus mediated methods.

Also included and provided for by the present disclosure methods to genetically engineer NKT cell cells to express two or more CAR molecules having antibody recognition domains that recognize two or more cancer antigens selected from the group consisting of MAGE, PRAME, CD19, CD20, CD22, K-light chain, CD30, CD33, CD123, CD38, CD138, RORl, ErbB2, ErbB3/4, EGFr viii, carcinoembryonic antigen, EGP2, EGP40, HER2, mesothelin, TAG72, PSMA, NKG2D ligands, B7-H6, IL-13 receptor a2, MUC1, MUC16, CA9, GD2, GD3, HMW-MAA, CD171, Lewis Y, G250/CAIX, HLA-AI MAGE Al, HLA-A2 NY-ESO-1, PSC1, folate receptor-a, CD44v6, CD44v7/8, 8H9, NCAM, VEGF receptors, 5T4, Fetal AchR, and CD44v6. Genetical engineering of NKT cells having two or more CAR molecules further includes constructs for expressing a Wnt signaling pathway protein and may further include sequences for expressing a growth factor selected from IL-2, IL-4, IL-7, IL-15, or IL-21. Expression constructs can be prepared separately, or more conveniently as one or more polyprotein expression constructs with CHYSEL sequences as described above. The methods of maintaining NKT cell expansion potential comprising introducing into NKT cells the expression constructs described herein, may further include isolating PMBCs from a subject, isolating NKT cells and expanding them by culture by stimulation with uGalCer-pulsed aAPCs in the presence of growth factors for between 1 and three days prior to introducing the expression constructs of the present disclosure. In certain aspects, the NKT cells separated by the methods according to the present disclosure are expanded in the presence of suitable stimulants (e.g., aGalCer, IL-2, and IL-21) for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, or at least 20 days to prepare the NKT cells for genetic engineering.

The methods of maintaining NKT cell expansion potential comprising introducing into NKT cells the expression constructs described herein, may further include isolating PMBCs from a subject, isolating NKT cells and expanding them by culture in the presence of suitable stimulants (e.g., aGalCer, IL-2, and IL-21) for between 1 to 20 days, 2 to 20 days, 3 to 20 days, 4 to 20 days, 5 to 20 days, 6 to 20 days, 7 to 20 days, 8 to 20 days, 9 to 20 days, 10 to 20 days, 11 to 20 days, 12 to 20 days, 13 to 20 days, or 14 to 20 days. In certain aspects, the NKT cells separated by the methods according to the present disclosure are expanded in culture in the presence of suitable stimulants (e.g., aGalCer, IL-2, and IL-21) prior to introducing the expression constructs described herein for about 1 day, about 2 days, about 3 days, about 4 days, about 5 days, about 6 days, about 7 days, about 8 days, about 9 days, about 10 days, about 11 days, or about 12 days.

In an aspects of the methods of maintaining NKT cell expansion potential, the CAR ectodomain sequences comprise and antibody recognition domain and a spacer domain.

In further aspects, the resulting genetically engineered NKT cells are expanded by culturing in the presence of CD1+ J32 leukemia cells. In aspects, the genetically engineered NKT cells are part of a population and the number of total cells in the population are determined and cultured together with antigen presenting cells, such as CD1+ J32 leukemia cells, and are provided at a ratio between 10:1 and 1:10 ratio. In an aspect, the antigen presenting cells, such as CD1+ J32 leukemia cells can be provided at a ratio of 1:1 relative to the total number of cells in the genetically engineered NKT cell population. In an aspect, the genetically engineered NKT cells are isolated prior to challenge. In other aspects, the genetically engineered NKT cells are cultured as part of a mixed population. In an aspect the mixed population comprises at least 10% genetically engineered NKT cells.

As provided herein, the genetically engineered NKT cells retain the ability to expand at least two fold after culture with antigen presenting cells, such as CD1+ J32 leukemia cells for more than three cycles of culture wherein each cycle is performed after a period of time. In aspects, the genetically engineered NKT cells retain the ability to expand at least two fold after culture with antigen presenting cells, such as CD1+ J32 leukemia cells for between three and 20 cycles of culture. In an aspect, the genetically engineered NKT cells retain the ability to expand at least two fold after culture with antigen presenting cells, such as CD1+ J32 leukemia cells for more than four cycles of culture. In an aspect, the genetically engineered NKT cells retain the ability to expand at least two fold after culture with antigen presenting cells, such as CD1+ J32 leukemia cells for more than five cycles of culture. In an aspect, the genetically engineered NKT cells retain the ability to expand at least two fold after culture with antigen presenting cells, such as CD1+ J32 leukemia cells for more than six cycles of culture. In an aspect, the genetically engineered NKT cells retain the ability to expand at least two fold after culture with antigen presenting cells, such as CD1+ J32 leukemia cells for more than seven cycles of culture. In an aspect, the genetically engineered NKT cells retain the ability to expand at least two fold after culture with antigen presenting cells, such as CD1+ J32 leukemia cells for more than eight cycles of culture. In an aspect, the genetically engineered NKT cells retain the ability to expand at least two fold after culture with antigen presenting cells, such as CD1+ J32 leukemia cells for more than nine cycles of culture.

As provided by the present disclosure, the genetically engineered NKT cells can be expanded repeatedly after a culture period of at least one day. In aspects, the expansion is performed after a period of between one and three days. In certain aspects, the genetically engineered NKT cells are expanded for a period of at least 1 day, at least 2 days, at least 3 days, at least 4 days, at least 5 days, at least 6 days, or at least 7 days before repeating the cycle with culture with CD1+ J32 leukemia cells or other antigen presenting cells.

The present disclosure further provides for performing one or more selections on a population of cells comprising a plurality of genetically engineered NKT cells. As provided herein, selection of genetically engineered NKT cell cells can be selection for both genetically engineered NKT cells and non-modified NKT cells and the selected population used for further culture and expansion. In aspects, the selection of genetically engineered NKT cell cells is a selection for genetically engineered NKT cells from a mixed population of cells that include both non-modified NKT cells and other cell types, usually lymphocytes. The selected populations are used for further culture and additional rounds expansion. In aspects, the selection and expansion of genetically engineered NKT cells can be performed multiple times, up to 20 times. The methods of the present disclosure thereby provide for the expansion of genetically engineered NKT cells of more than a million fold. In aspects, the methods provide for selection and expansion at least three times. The present disclosure further provides for, and includes, selection and expansion at least four times, at least five times, and at least six times. In additional aspects, the genetically engineered NKT cells are selected and expanded for at least 7 times. In further aspects, the genetically engineered NKT cells are selected and expanded for eight, nine or more times.

The present disclosure further provides for performing one or more selections on a population of cells comprising a plurality of genetically engineered NKT cells. As provided herein, selection of genetically engineered NKT cell cells can be selection for Type I NKT cells having persistent expansion potential followed by expansion.

In aspects provided herein, the selected Type I genetically engineered NKT cells populations are used for further culture and additional rounds expansion. In aspects, the selection and expansion of Type I genetically engineered NKT cells can be performed multiple times, up to 20 times. The methods of the present disclosure thereby provide for the expansion of Type I genetically engineered NKT cells of more than a million fold. In aspects, the methods provide for selection and expansion at least three times. The present disclosure further provides for, and includes, selection and expansion at least four times, at least five times, and at least six times. In additional aspects, the Type I genetically engineered NKT cells are selected and expanded for at least 7 times. In further aspects, the Type I genetically engineered NKT cells are selected and expanded for eight, nine or more times.

Also provided for, and included is the expansion of Type I genetically engineered NKT cells to produce populations that comprise at least 20% of the total population of cells. In an aspect the Type I genetically engineered NKT cells comprise at least 20% of the total population of cells after at least two rounds of expansion.

Provided for, and included in, the present disclosure are methods for maintaining NKT cell expansion potential comprising the steps of engineering NKT cells to express at least a protein coding sequence comprising a transcriptional activator in the Wnt signaling pathway and culturing the engineered NKT cells to prepare a population of genetically engineered NKT cells with persistent expansion potential to produce cell populations wherein the genetically engineered NKT cells comprise greater than 10% of the total population of cells. In aspects, the 10% or greater population of genetically engineered NKT cells with persistent expansion potential comprises Type I NKT cells. In aspects, the total population comprises Type I NKT cells, Type II NKT cells, irradiated PBMC cells, non-NKT cells, and non-engineered cells. In aspects, the engineered NKT cells further express a CAR. In other aspects, the engineered NKT cells express a CAR and an exogenous growth factor.

In certain aspects, the population of genetically engineered NKT cells with persistent expansion potential according to the present disclosure comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the total cell population. In certain aspects, the population of genetically engineered NKT cells with persistent expansion potential according to the present disclosure comprises at least 10% up to 80%, between 10% and 90%, between 10% and 95%, between 10% and 98%, between 10% and 99%, and up to 100% wherein non-engineered NKT cells comprise less than 99.9% of the total population. In aspects, the population of genetically engineered NKT cells with persistent expansion potential according to the present disclosure comprises at least 50%, at least 55%, at least 60%, at least 65%, at least 70%, at least 75%, at least 80%, at least 85%, at least 90%, or at least 95% of the total cell population. In certain aspects, the population of genetically engineered NKT cells with persistent expansion potential according to the present disclosure comprises between at least 50% up to 70%, between 50% up to 80%, between 50% and 90%, between 50% and 95%, between 50% and 98%, between 50% and 99%, and up to 100% wherein non-engineered NKT cells comprise less than 99.9% of the total population. In aspects, the engineered NKT cells further express a CAR. In other aspects, the engineered NKT cells express a CAR and an exogenous growth factor.

The present disclosure provides for, and includes, methods for maintaining NKT cell expansion potential comprising the steps of engineering NKT cells to express at least a protein coding sequence comprising a transcriptional activator in the Wnt signaling pathway and culturing the engineered NKT cells to prepare a population of genetically engineered NKT cells with persistent expansion potential to produce cell populations wherein the genetically engineered NKT cells comprise greater than 10% CD62L(+) NKT cells of the total population of cells. In aspects, the 10% or greater population of genetically engineered CD62L(+) NKT cells with persistent expansion potential comprises Type I NKT cells. In aspects, the total population comprises Type I NKT cells, Type II NKT cells, irradiated PBMC cells, non-NKT cells, and non-engineered cells. In aspects, the engineered CD62L(+) NKT cells further express a CAR. In other aspects, the engineered CD62L(+) NKT cells express a CAR and an exogenous growth factor.

In certain aspects, the population of genetically engineered NKT cells with persistent expansion potential according to the present disclosure comprises at least 50%, at least 55%, at least 60%, at least 65%, at least 70%, at least 75%, at least 80%, at least 85%, at least 90%, or at least 95% CD62L(+) NKT cells. In certain aspects, the population of genetically engineered NKT cells with persistent expansion potential according to the present disclosure comprises about 50% to about 55%, about 50% to about 60%, about 50% to about 65%, about 50% to about 70%, about 50% to about 75%, about 50% to about 80%, about 50% to about 85%, about 50% to about 90%, about 50% to about 95%, about 50% to 100%, about 55% to about 60%, about 55% to about 65%, about 55% to about 70%, about 55% to about 75%, about 55% to about 80%, about 55% to about 85%, about 55% to about 90%, about 55% to about 95%, about 55% to 100%, about 60% to about 65%, about 60% to about 70%, about 60% to about 75%, about 60% to about 80%, about 60% to about 85%, about 60% to about 90%, about 60% to about 95%, about 60% to 100%, about 65% to about 70%, about 65% to about 75%, about 65% to about 80%, about 65% to about 85%, about 65% to about 90%, about 65% to about 95%, about 65% to 100%, about 70% to about 75%, about 70% to about 80%, about 70% to about 85%, about 70% to about 90%, about 70% to about 95%, about 70% to 100%, about 75% to about 80%, about 75% to about 85%, about 75% to about 90%, about 75% to about 95%, about 75% to 100%, about 80% to about 85%, about 80% to about 90%, about 80% to about 95%, about 80% to 100%, about 85% to about 90%, about 85% to about 95%, about 85% to 100%, about 90% to about 95%, about 90% to 100%, or about 95% to 100% CD62L(+) NKT cells. In certain aspects, the population of genetically engineered NKT cells with persistent expansion potential according to the present disclosure comprises about 50%, about 55%, about 60%, about 65%, about 70%, about 75%, about 80%, about 85%, about 90%, about 95%, or 100% CD62L(+) NKT cells. In aspects, the engineered CD62L(+) NKT cells further express a CAR. In other aspects, the engineered CD62L(+) NKT cells express a CAR and an exogenous growth factor.

The present disclosure provides for, and includes, methods for maintaining NKT cell expansion potential comprising the steps of engineering NKT cells to express at least a protein coding sequence comprising a transcriptional activator in the Wnt signaling pathway and culturing the engineered NKT cells to prepare a population of genetically engineered NKT cells with persistent expansion potential, wherein said engineered NKT cells are cultured for a period of time. In an aspect, the culture period is for about 1 to 2 days, about 1 to 3 days, about 1 to 4 days, about 1 to 5 days, about 1 to 6 days, about 1 to 7 days, about 1 to 8 days, about 1 to 9 days, about 1 to 10 days, about 1 to 11 days, about 1 to 12 days, about 1 to 13 days, about 1 to 14 days, about 1 to 15 days, about 1 to 16 days, about 1 to 17 days, about 1 to 18 days, about 1 to 19 days, about 1 to 20 days, about 2 to 3 days, about 2 to 4 days, about 2 to 5 days, about 2 to 6 days, about 2 to 7 days, about 2 to 8 days, about 2 to 9 days, about 2 to 10 days, about 2 to 11 days, about 2 to 12 days, about 2 to 13 days, about 2 to 14 days, about 2 to 15 days, about 2 to 16 days, about 2 to 17 days, about 2 to 18 days, about 2 to 19 days, about 2 to 20 days, about 3 to 4 days, about 3 to 5 days, about 3 to 6 days, about 3 to 7 days, about 3 to 8 days, about 3 to 9 days, about 3 to 10 days, about 3 to 11 days, about 3 to 12 days, about 3 to 13 days, about 3 to 14 days, about 3 to 15 days, about 3 to 16 days, about 3 to 17 days, about 3 to 18 days, about 3 to 19 days, about 3 to 20 days, about 4 to 5 days, about 4 to 6, about 4 to 7, about 4 to 8, about 4 to 9, about 4 to 10, about 4 to 11, about 4 to 12, about 4 to 13 days, about 4 to 14 days, about 4 to 15 days, about 4 to 16 days, about 4 to 17 days, about 4 to 18 days, about 4 to 19 days, about 4 to 20 days, about 5 to 6 days, about 5 to 7 days, about 5 to 8 days, about 5 to 9 days, about 5 to 10 days, about 5 to 11 days, about 5 to 12 days, about 5 to 13 days, about 5 to 14 days, about 5 to 15 days, about 5 to 16 days, about 5 to 17 days, about 5 to 18 days, about 5 to 19 days, about to 20 days, about 6 to 7 days, about 6 to 8 days, about 6 to 9 days, about 6 to 10 days, about 6 to 11 days, about 6 to 12 days, about 6 to 13 days, about 6 to 14 days, about 6 to 15 days, about 6 to 16 days, about 6 to 17 days, about 6 to 18 days, about 6 to 19 days, about 6 to 20 days, about 7 to 8 days, about 7 to 9 days, about 7 to 10 days, about 7 to 11 days, about 7 to 12 days, about 7 to 13, about 7 to 14, about 7 to 15, about 7 to 16, about 7 to 17, about 7 to 18, about 7 to 19 days, about 7 to 20 days, about 8 to 9 days, about 8 to 10 days, about 8 to 11 days, about 8 to 12 days, about 8 to 13 days, about 8 to 14 days, about 8 to 15, days about 8 to 16 days, about 8 to 17 days, about 8 to 18 days, about 8 to 19 days, about 8 to 20 days, about 9 to 10 days, about 9 to 11 days, about 9 to 12 days, about 9 to 13 days, about 9 to 14 days, about 9 to 15, days about 9 to 16 days, about 9 to 17 days, about 9 to 18 days, about 9 to 19 days, about 9 to 20 days, about 10 to 11 days, about 10 to 12 days, about 10 to 13 days, about 10 to 14 days, about to 15 days, about 10 to 16 days, about 10 to 17 days, about 10 to 18 days, about 10 to 19 days, about 10 to 20 days, about 11 to 12 days, about 11 to 13 days, about 11 to 14 days, about 11 to 15 days, about 11 to 16 days, about 11 to 17 days, about 11 to 18 days, about 11 to 19 days, about 11 to 20 days, about 12 to 13 days, about 12 to 14 days, about 12 to 15 days, about 12 to 16 days, about 12 to 17 days, about 12 to 18 days, about 12 to 19 days, about 12 to 20 days, about 13 to 14 days, about 13 to 15 days, about 13 to 16 days, about 13 to 17 days, about 13 to 18 days, about 13 to 19 days, about 13 to 20 days, about 14 to 15 days, about 14 to 16 days, about 14 to 17 days, about 14 to 18 days, about 14 to 19 days, about 14 to 20 days, about to 16 days, about 15 to 17 days, about 15 to 18 days, about 15 to 19 days, about 15 to 20 days, about 16 to 17 days, about 16 to 18 days, about 16 to 19 days, about 16 to 20 days, about 17 to 18 days, about 17 to 19 days, about 17 to 20 days, about 18 to 19 days, about 18 to 20 days, or about 19 to 20 days to prepare a population of genetically engineered NKT cells with persistent expansion potential. In aspects, the engineered NKT cells further express a CAR. In other aspects, the engineered NKT cells express a CAR and an exogenous growth factor.

In aspects, the methods for maintaining NKT cell expansion potential comprising the steps of engineering NKT cells further comprises separating desired cells from the population of genetically engineered NKT cells with persistent expansion potential. In an aspect, the method further comprises separating the engineered NKT cells by the expression of CD62L to produce a selected population of CD62L(+) genetically engineered NKT cells. In an aspect, the method further comprises separating the engineered NKT cells by the expression of 4-1BB to produce a selected population of 4-1BB (+) genetically engineered NKT cells.

The present disclosure provides for, and includes, methods for maintaining NKT cell expansion potential comprising the steps of isolating fresh PBMCs from a donor, separating NKT cells from the PBMCs prior to culture, expanding said separated NKT cells in the presence of at least aGalCer, and a growth factor for at least 1 day to prepare NKT cells for genetic engineering. In an aspect, the growth factor comprises IL-7, IL-12, IL-15, IL-21, TNF-alpha, or a combination thereof. In an aspect, the steps of preparing NKT cells for genetic engineering further comprises separating the NKT cells from PBMCs using anti-NKT microbeads.

The methods for maintaining NKT cell expansion potential of the present disclosure provides for, and includes, preparing genetically engineered cell populations that can be further expanded at least two fold by challenge with antigen presenting cells. In an aspect, the antigen presenting cells are CDld+ J32 leukemia cells. In aspects, the antigen presenting cells are provided at a ratio between 10:1 and 1:10 ratio. In an aspect, the challenge with antigen presenting cells is performed at a 1:1 ratio. In aspects, the antigen presenting cells are CD1d+ J32 leukemia cells.

The methods for maintaining NKT cell expansion potential of the present disclosure provides for, and includes, populations of genetically engineered NKT cells with persistent expansion potential exhibit in vivo persistence as infiltrates into neuroblastorna xenografts in humanized NSG mice.

The present disclosure provides for, and includes, methods for reducing NKT cell exhaustion in an NKT cell population comprising the steps of engineering NKT cells to express at least a protein coding sequence comprising a transcriptional activator in the Wnt signaling pathway and culturing the engineered NKT cells to prepare a population of genetically engineered NKT cells with reduced NKT cell exhaustion. In an aspect, the method further provides for isolating PBMCs from a donor, separating NKT cells from the PBMCs, and expanding the separated NKT cells in the presence of at least αGalCer, IL-2, and IL-21 for at least 1 day to prepare NKT cells for genetic engineering. The cells are then transfected or transduced with the expression vectors described herein and cultured to prepare NKT cells with reduced NKT cell exhaustion.

In aspects, the methods for reducing NKT cell exhaustion in an NKT cell population provide for expression of a transcriptional activator in the Wnt signaling pathway that includes, but is not limited to lymphoid enhancer binding factor 1 (LEF1, Gene ID 51176), beta-catenin ((CTNNB1, Gene ID 1499)), Smad3 (Gene ID 4088), and TLE 1 (Gene ID 7088). In an aspect, the genetically engineered NKT cell comprises a expression construct encoding LEF1 is selected from the group consisting of Reference Sequence (RefSeq) ID NOs: NP_057353.1, NP_001124185.1, and NP_001124186.1. In an aspect, the genetically engineered NKT cell expresses LEF1 and a growth factor. In an aspect, the expression construct further encodes a growth factor selected from the group consisting of IL-15, IL-2, IL-4, IL-7, and combinations thereof.

In certain aspects, the NKT cells separated by the methods according to the present disclosure are expanded in the presence of suitable stimulants (e.g., αGalCer, IL-2, and IL-21) for between 1 and 20 days as provided herein.

Also included, and provided, by the present disclosure are methods for reducing NKT cell exhaustion in an NKT cell population comprising transfecting or transducing NKT cells with an expression construct comprising (a) a chimeric antigen receptor (CAR) coding sequence comprising an ectodomain sequence, a transmembrane domain sequence, and an endodomain sequence and (b) a sequence encoding a protein sequence for a transcriptional activator in the Wnt signaling pathway to prepare a genetically engineered NKT cell.

In aspects, the methods for reducing NKT cell exhaustion in an NKT cell population provide for expression of a transcriptional activator in the Wnt signaling pathway that includes, but is not limited to lymphoid enhancer binding factor 1 (LEF1, Gene ID 51176), beta-catenin ((CTNNB1, Gene ID 1499)), Smad3 (Gene ID 4088), and TLE 1 (Gene ID 7088). In an aspect, the genetically engineered NKT cell comprises a expression construct encoding LEF1 is selected from the group consisting of Reference Sequence (RefSeq) ID NOs: NP_057353.1, NP_001124185.1, and NP_001124186.1. In an aspect, the genetically engineered NKT cell expresses LEF1 and a growth factor. In an aspect, the expression construct further encodes a growth factor selected from the group consisting of IL-15, IL-2, IL-4, IL-7, and combinations thereof.

In certain aspects, the methods for reducing NKT cell exhaustion in an NKT cell population includes genetically engineering NKT cells to comprise at least a CAR in combination with expression of a transcriptional activator in the Wnt signaling pathway. In specific aspects, NKT cells are engineered to express two or more CARs in combination with expression of a transcriptional activator in the Wnt signaling pathway. In an aspect, genetically engineering the NKT cell comprises introducing an expression construct for a transcriptional activator in the Wnt signaling pathway selected from the group consisting of lymphoid enhancer binding factor 1 (LEF1, Gene ID 51176), beta-catenin (CTNNB1), Smad3, and TLE 1. In and aspect, the transcriptional activator is LEF1 selected from the group consisting of Reference Sequence (RefSeq) ID NOs: NP_057353.1, NP_001124185.1, NP_001124186.1 in combination with one or more CARs.

Included, and provided by the methods for reducing NKT cell exhaustion in an NKT cell population are expression of CARs recognizing cancer antigens such as Melanoma-associated antigen (MAGE), Preferentially expressed antigen of melanoma (PRAME), CD19, CD20, CD22, K-light chain, CD30, CD33, CD123, CD38, CD138, RORI, ErbB2, ErbB3/4, EGFr vIII, carcinoembryonic antigen, EGP2, EGP40, HER2, mesothelin, TAG72, PSMA, NKG2D ligands, B7-H6, IL-13 receptor a2, MUC1, MUC16, CA9, GD2, GD3, HMW-MAA, CD171, Lewis Y, G250/CAIX, HLA-AI MAGE Al, HLA-A2 NY-ESO-1, PSC1, folate receptor-a, CD44v6, CD44v7/8, 8H9, NCAM, VEGF receptors, 5T4, Fetal AchR, or CD44v6. In an aspect, the cancer antigens introduced to genetically engineer NKT cells are selected from the group consisting of CD19, GD2, and GPC3. In another aspect, the antibody recognition domain of the CAR recognized the cancer antigen CD19. In an aspect, the cancer antigen recognized by the antibody recognition domain is GD2. In yet another aspect, the cancer antigen recognized by the antibody recognition domain GPC3. Methods to introduce expression constructs are known in the art. In aspects or the present disclosure the genetic engineering includes and provides for introducing the constructs of the present disclosure using retrovirus or lentivirus mediated methods.

Also included and provided for by the present disclosure methods to genetically engineer NKT cell cells to express two or more CAR molecules having antibody recognition domains that recognize two or more cancer antigens selected from the group consisting of MAGE, PRAME, CD19, CD20, CD22, K-light chain, CD30, CD33, CD123, CD38, CD138, RORl, ErbB2, ErbB3/4, EGFr viii, carcinoembryonic antigen, EGP2, EGP40, HER2, mesothelin, TAG72, PSMA, NKG2D ligands, B7-H6, IL-13 receptor a2, MUC1, MUC16, CA9, GD2, GD3, HMW-MAA, CD171, Lewis Y, G250/CAIX, HLA-AI MAGE Al, HLA-A2 NY-ESO-1, PSC1, folate receptor-a, CD44v6, CD44v7/8, 8H9, NCAM, VEGF receptors, 5T4, Fetal AchR, and CD44v6. Genetical engineering of NKT cells having two or more CAR molecules further includes constructs for expressing a Wnt signaling pathway protein and may further include sequences for expressing a growth factor selected from IL-2, IL-4, IL-7, IL-15, or IL-21. Expression constructs can be prepared separately, or more conveniently as one or more polyprotein expression constructs with CHYSEL sequences as described above. The methods of maintaining NKT cell expansion potential comprising introducing into NKT cells the expression constructs described herein, may further include isolating PMBCs from a subject, isolating NKT cells and expanding them by culture by stimulation with uGalCer-pulsed aAPCs in the presence of growth factors for between 1 and three days prior to introducing the expression constructs of the present disclosure. In certain aspects, the NKT cells separated by the methods according to the present disclosure are expanded in the presence of suitable stimulants (e.g., αGalCer, IL-2, and IL-21) for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, or at least 20 days to prepare the NKT cells for genetic engineering.

The methods for reducing NKT cell exhaustion in an NKT cell population comprising introducing into NKT cells the expression constructs described herein, may further include isolating PMBCs from a subject, isolating NKT cells and expanding them by culture in the presence of suitable stimulants (e.g., aGalCer, IL-2, and IL-21) for between 1 to 20 days, 2 to 20 days, 3 to 20 days, 4 to 20 days, 5 to 20 days, 6 to 20 days, 7 to 20 days, 8 to 20 days, 9 to 20 days, 10 to 20 days, 11 to 20 days, 12 to 20 days, 13 to 20 days, or 14 to 20 days. In certain aspects, the NKT cells separated by the methods according to the present disclosure are expanded in culture in the presence of suitable stimulants (e.g., aGalCer, IL-2, and IL-21) prior to introducing the expression constructs described herein for about 1 day, about 2 days, about 3 days, about 4 days, about 5 days, about 6 days, about 7 days, about 8 days, about 9 days, about 10 days, about 11 days, or about 12 days.

In an aspects of the methods for reducing NKT cell exhaustion in an NKT cell population, the CAR ectodomain sequences comprise an antibody recognition domain and a spacer domain.

In further aspects, resulting populations of genetically engineered NKT cells with reduced NKT cell exhaustion can be expanded by culturing in the presence of antigen presenting cells, such as CD1+ J32 leukemia cells or aAPCs. In aspects, the genetically engineered NKT cells are part of a population and the number of total cells in the population are determined and cultured together with antigen presenting cells, such as CD1+ J32 leukemia cells, and are provided at a ratio between 10:1 and 1:10 ratio. In an aspect, the antigen presenting cells, such as CD1+ J32 leukemia cells can be provided at a ratio of 1:1 relative to the total number of cells in the genetically engineered NKT cell population. In an aspect, the genetically engineered NKT cells are isolated prior to challenge. In other aspects, the genetically engineered NKT cells are cultured as part of a mixed population. In an aspect the mixed population comprises at least 10% genetically engineered NKT cells.

As provided herein, the populations of genetically engineered NKT cells with reduced NKT cell exhaustion retain the ability to expand at least two fold after culture with antigen presenting cells, such as CD1+ J32 leukemia cells for more than three cycles of culture wherein each cycle is performed after a period of time. In aspects, the genetically engineered NKT cells retain the ability to expand at least two fold after culture with antigen presenting cells, such as CD1+ J32 leukemia cells for between three and 20 cycles of culture. In an aspect, the genetically engineered NKT cells retain the ability to expand at least two fold after culture with antigen presenting cells, such as CD1+ J32 leukemia cells for more than four cycles of culture. In an aspect, the genetically engineered NKT cells retain the ability to expand at least two fold after culture with antigen presenting cells, such as CD1+ J32 leukemia cells for more than five cycles of culture. In an aspect, the genetically engineered NKT cells retain the ability to expand at least two fold after culture with antigen presenting cells, such as CD1+ J32 leukemia cells for more than six cycles of culture. In an aspect, the genetically engineered NKT cells retain the ability to expand at least two fold after culture with antigen presenting cells, such as CD1+ J32 leukemia cells for more than seven cycles of culture. In an aspect, the genetically engineered NKT cells retain the ability to expand at least two fold after culture with antigen presenting cells, such as CD1+ J32 leukemia cells for more than eight cycles of culture. In an aspect, the genetically engineered NKT cells retain the ability to expand at least two fold after culture with antigen presenting cells, such as CD1+ J32 leukemia cells for more than nine cycles of culture.

As provided by the present disclosure, the genetically engineered NKT cells can be expanded repeatedly after a culture period of at least one day. In aspects, the expansion is performed after a period of between one and three days. In certain aspects, the genetically engineered NKT cells are expanded for a period of at least 1 day, at least 2 days, at least 3 days, at least 4 days, at least 5 days, at least 6 days, or at least 7 days before repeating the cycle with culture with CD1+ J32 leukemia cells or other antigen presenting cells.

The present disclosure further provides for performing one or more selections on a population of cells comprising a plurality of genetically engineered NKT cells with reduced NKT cell exhaustion. As provided herein, selection of genetically engineered NKT cell cells can be selection for both genetically engineered NKT cells and non-modified NKT cells and the selected population used for further culture and expansion. In aspects, the selection of genetically engineered NKT cell cells is a selection for genetically engineered NKT cells from a mixed population of cells that include both non-modified NKT cells and other cell types, usually lymphocytes. The selected populations are used for further culture and additional rounds expansion. In aspects, the selection and expansion of genetically engineered NKT cells can be performed multiple times, up to 20 times. The methods of the present disclosure thereby provide for the expansion of genetically engineered NKT cells of more than a million fold. In aspects, the methods provide for selection and expansion at least three times. The present disclosure further provides for, and includes, selection and expansion at least four times, at least five times, and at least six times. In additional aspects, the genetically engineered NKT cells are selected and expanded for at least 7 times. In further aspects, the genetically engineered NKT cells are selected and expanded for eight, nine or more times.

The present disclosure further provides for performing one or more selections on a population of cells comprising a plurality of genetically engineered NKT cells with reduced NKT cell exhaustion. As provided herein, selection of genetically engineered NKT cell cells can be selection for Type I NKT cells having reduced NKT cell exhaustion followed by expansion.

In aspects provided herein, selected Type I genetically engineered NKT cell populations are used for further culture and additional rounds expansion. In aspects, the selection and expansion of Type I genetically engineered NKT cells can be performed multiple times, up to 20 times. The methods of the present disclosure thereby provide for the expansion of Type I genetically engineered NKT cells of more than a million fold. In aspects, the methods provide for selection and expansion at least three times. The present disclosure further provides for, and includes, selection and expansion at least four times, at least five times, and at least six times. In additional aspects, the Type I genetically engineered NKT cells are selected and expanded for at least 7 times. In further aspects, the Type I genetically engineered NKT cells are selected and expanded for eight, nine or more times.

Also provided for, and included is the expansion of Type I genetically engineered NKT cells to produce populations that comprise at least 20% of the total population of cells. In an aspect the Type I genetically engineered NKT cells comprise at least 20% of the total population of cells after at least two rounds of expansion.

Provided for, and included in, the present disclosure are methods for reducing NKT cell exhaustion in an NKT cell population comprising the steps of engineering NKT cells to express at least a protein coding sequence comprising a transcriptional activator in the Wnt signaling pathway and culturing the engineered NKT cells to prepare a population of genetically engineered NKT cells with persistent expansion potential to produce cell populations wherein the genetically engineered NKT cells comprise greater than 10% of the total population of cells. In aspects, the 10% or greater population of genetically engineered NKT cells with persistent expansion potential comprises Type I NKT cells. In aspects, the total population comprises Type I NKT cells, Type II NKT cells, irradiated PBMC cells, non-NKT cells, and non-engineered cells. In aspects, the engineered NKT cells further express a CAR. In other aspects, the engineered NKT cells express a CAR and an exogenous growth factor.

In certain aspects, the population of genetically engineered NKT cells with reduced NKT cell exhaustion within an NKT cell population according to the present disclosure comprises at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the total cell population. In certain aspects, the population of genetically engineered NKT cells with reduced NKT cell exhaustion according to the present disclosure comprises about 10% to about 15%, about 10% to about 20%, about 10% to about 25%, about 10% to about 30%, about 10% to about 35%, about 10% to about 40%, about 10% to about 45%, about 10% to about 50%, about 10% to about 55%, about 10% to about 60%, about 10% to about 65%, about 10% to about 70%, about 10% to about 75%, about 10% to about 80%, about 10% to about 85%, about 10% to about 90%, about 10% to about 95%, about 10% to 100%, about 15% to about 20%, about 15% to about 25%, about 15% to about 30%, about 15% to about 35%, about 15% to about 40%, about 15% to about 45%, about 15% to about 50%, about 15% to about 55%, about 15% to about 60%, about 15% to about 65%, about 15% to about 70%, about 15% to about 75%, about 15% to about 80%, about 15% to about 85%, about 15% to about 90%, about 15% to about 95%, about 15% to 100%, about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 20% to about 80%, about 20% to about 85%, about 20% to about 90%, about 20% to about 95%, about 20% to 100%, about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 25% to about 80%, about 25% to about 85%, about 25% to about 90%, about 25% to about 95%, about 25% to 100%, about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 30% to about 80%, about 30% to about 85%, about 30% to about 90%, about 30% to about 95%, about 30% to 100%, about 35% to about 40%, about 35% to about 45%, about 35% to about 50%, about 35% to about 55%, about 35% to about 60%, about 35% to about 65%, about 35% to about 70%, about 35% to about 75%, about 35% to about 80%, about 35% to about 85%, about 35% to about 90%, about 35% to about 95%, about 35% to 100%, about 40% to about 45%, about 40% to about 50%, about 40% to about 55%, about 40% to about 60%, about 40% to about 65%, about 40% to about 70%, about 40% to about 75%, about 40% to about 80%, about 40% to about 85%, about 40% to about 90%, about 40% to about 95%, about 40% to 100%, about 45% to about 50%, about 45% to about 55%, about 45% to about 60%, about 45% to about 65%, about 45% to about 70%, about 45% to about 75%, about 45% to about 80%, about 45% to about 85%, about 45% to about 90%, about 45% to about 95%, about 45% to 100%, about 50% to about 55%, about 50% to about 60%, about 50% to about 65%, about 50% to about 70%, about 50% to about 75%, about 50% to about 80%, about 50% to about 85%, about 50% to about 90%, about 50% to about 95%, about 50% to 100%, about 55% to about 60%, about 55% to about 65%, about 55% to about 70%, about 55% to about 75%, about 55% to about 80%, about 55% to about 85%, about 55% to about 90%, about 55% to about 95%, about 55% to 100%, about 60% to about 65%, about 60% to about 70%, about 60% to about 75%, about 60% to about 80%, about 60% to about 85%, about 60% to about 90%, about 60% to about 95%, about 60% to 100%, about 65% to about 70%, about 65% to about 75%, about 65% to about 80%, about 65% to about 85%, about 65% to about 90%, about 65% to about 95%, about 65% to 100%, about 70% to about 75%, about 70% to about 80%, about 70% to about 85%, about 70% to about 90%, about 70% to about 95%, about 70% to 100%, about 75% to about 80%, about 75% to about 85%, about 75% to about 90%, about 75% to about 95%, about 75% to 100%, about 80% to about 85%, about 80% to about 90%, about 80% to about 95%, about 80% to 100%, about 85% to about 90%, about 85% to about 95%, about 85% to 100%, about 90% to about 95%, about 90% to 100%, or about 95% to 100% of the total cell population. In certain aspects, the population of genetically engineered NKT cells with reduced NKT cell exhaustion according to the present disclosure comprises about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or 100% of the total cell population. In aspects, the engineered NKT cells further express a CAR. In other aspects, the engineered NKT cells express a CAR and an exogenous growth factor.

The present disclosure provides for, and includes, methods for reducing NKT cell exhaustion comprising the steps of engineering NKT cells to express at least a protein coding sequence comprising a transcriptional activator in the Wnt signaling pathway and culturing the engineered NKT cells to prepare a population of genetically engineered NKT cells with persistent expansion potential to produce cell populations wherein the genetically engineered NKT cells comprise greater than 10% CD62L(+) NKT cells of the total population of cells. In aspects, the 10% or greater population of genetically engineered CD62L(+) NKT cells with persistent expansion potential comprises Type I NKT cells. In aspects, the total population comprises Type I NKT cells, Type II NKT cells, irradiated PBMC cells, non-NKT cells, and non-engineered cells. In aspects, the engineered CD62L(+) NKT cells further express a CAR. In other aspects, the engineered CD62L(+) NKT cells express a CAR and an exogenous growth factor.

In certain aspects, the population of genetically engineered NKT cells with reduced NKT cell exhaustion within an NKT cell population according to the present disclosure comprises at least 50%, at least 55%, at least 60%, at least 65%, at least 70%, at least 75%, at least 80%, at least 85%, at least 90%, or at least 95% CD62L(+) NKT cells. In certain aspects, the population of genetically engineered NKT cells with reduced NKT cell exhaustion according to the present disclosure comprises about 50% to about 55%, about 50% to about 60%, about 50% to about 65%, about 50% to about 70%, about 50% to about 75%, about 50% to about 80%, about 50% to about 85%, about 50% to about 90%, about 50% to about 95%, about 50% to 100%, about 55% to about 60%, about 55% to about 65%, about 55% to about 70%, about 55% to about 75%, about 55% to about 80%, about 55% to about 85%, about 55% to about 90%, about 55% to about 95%, about 55% to 100%, about 60% to about 65%, about 60% to about 70%, about 60% to about 75%, about 60% to about 80%, about 60% to about 85%, about 60% to about 90%, about 60% to about 95%, about 60% to 100%, about 65% to about 70%, about 65% to about 75%, about 65% to about 80%, about 65% to about 85%, about 65% to about 90%, about 65% to about 95%, about 65% to 100%, about 70% to about 75%, about 70% to about 80%, about 70% to about 85%, about 70% to about 90%, about 70% to about 95%, about 70% to 100%, about 75% to about 80%, about 75% to about 85%, about 75% to about 90%, about 75% to about 95%, about 75% to 100%, about 80% to about 85%, about 80% to about 90%, about 80% to about 95%, about 80% to 100%, about 85% to about 90%, about 85% to about 95%, about 85% to 100%, about 90% to about 95%, about 90% to 100%, or about 95% to 100% CD62L(+) NKT cells. In certain aspects, the population of genetically engineered NKT cells with reduced NKT cell exhaustion according to the present disclosure comprises about 50%, about 55%, about 60%, about 65%, about 70%, about 75%, about 80%, about 85%, about 90%, about 95%, or 100% CD62L(+) NKT cells. In aspects, the engineered CD62L(+) NKT cells further express a CAR. In other aspects, the engineered CD62L(+) NKT cells express a CAR and an exogenous growth factor.

The present disclosure provides for, and includes, methods for reducing NKT cell exhaustion within an NKT cell population comprising the steps of engineering NKT cells to express at least a protein coding sequence comprising a transcriptional activator in the Wnt signaling pathway and culturing the engineered NKT cells to prepare a population of genetically engineered NKT cells with reduced NKT cell exhaustion, wherein said engineered NKT cells are cultured for a period of time. In an aspect, the culture period is for about 1 to 2 days, about 1 to 3 days, about 1 to 4 days, about 1 to 5 days, about 1 to 6 days, about 1 to 7 days, about 1 to 8 days, about 1 to 9 days, about 1 to 10 days, about 1 to 11 days, about 1 to 12 days, about 1 to 13 days, about 1 to 14 days, about 1 to 15 days, about 1 to 16 days, about 1 to 17 days, about 1 to 18 days, about 1 to 19 days, about 1 to 20 days, about 2 to 3 days, about 2 to 4 days, about 2 to 5 days, about 2 to 6 days, about 2 to 7 days, about 2 to 8 days, about 2 to 9 days, about 2 to 10 days, about 2 to 11 days, about 2 to 12 days, about 2 to 13 days, about 2 to 14 days, about 2 to 15 days, about 2 to 16 days, about 2 to 17 days, about 2 to 18 days, about 2 to 19 days, about 2 to 20 days, about 3 to 4 days, about 3 to 5 days, about 3 to 6 days, about 3 to 7 days, about 3 to 8 days, about 3 to 9 days, about 3 to 10 days, about 3 to 11 days, about 3 to 12 days, about 3 to 13 days, about 3 to 14 days, about 3 to 15 days, about 3 to 16 days, about 3 to 17 days, about 3 to 18 days, about 3 to 19 days, about 3 to 20 days, about 4 to 5 days, about 4 to 6, about 4 to 7, about 4 to 8, about 4 to 9, about 4 to 10, about 4 to 11, about 4 to 12, about 4 to 13 days, about 4 to 14 days, about 4 to 15 days, about 4 to 16 days, about 4 to 17 days, about 4 to 18 days, about 4 to 19 days, about 4 to 20 days, about 5 to 6 days, about 5 to 7 days, about 5 to 8 days, about 5 to 9 days, about 5 to 10 days, about 5 to 11 days, about 5 to 12 days, about 5 to 13 days, about 5 to 14 days, about 5 to 15 days, about 5 to 16 days, about 5 to 17 days, about 5 to 18 days, about to 19 days, about 5 to 20 days, about 6 to 7 days, about 6 to 8 days, about 6 to 9 days, about 6 to 10 days, about 6 to 11 days, about 6 to 12 days, about 6 to 13 days, about 6 to 14 days, about 6 to 15 days, about 6 to 16 days, about 6 to 17 days, about 6 to 18 days, about 6 to 19 days, about 6 to 20 days, about 7 to 8 days, about 7 to 9 days, about 7 to 10 days, about 7 to 11 days, about 7 to 12 days, about 7 to 13, about 7 to 14, about 7 to 15, about 7 to 16, about 7 to 17, about 7 to 18, about 7 to 19 days, about 7 to 20 days, about 8 to 9 days, about 8 to 10 days, about 8 to 11 days, about 8 to 12 days, about 8 to 13 days, about 8 to 14 days, about 8 to 15, days about 8 to 16 days, about 8 to 17 days, about 8 to 18 days, about 8 to 19 days, about 8 to 20 days, about 9 to 10 days, about 9 to 11 days, about 9 to 12 days, about 9 to 13 days, about 9 to 14 days, about 9 to 15, days about 9 to 16 days, about 9 to 17 days, about 9 to 18 days, about 9 to 19 days, about 9 to 20 days, about 10 to 11 days, about 10 to 12 days, about 10 to 13 days, about 10 to 14 days, about 10 to 15 days, about 10 to 16 days, about 10 to 17 days, about 10 to 18 days, about 10 to 19 days, about 10 to 20 days, about 11 to 12 days, about 11 to 13 days, about 11 to 14 days, about 11 to 15 days, about 11 to 16 days, about 11 to 17 days, about 11 to 18 days, about 11 to 19 days, about 11 to 20 days, about 12 to 13 days, about 12 to 14 days, about 12 to 15 days, about 12 to 16 days, about 12 to 17 days, about 12 to 18 days, about 12 to 19 days, about 12 to 20 days, about 13 to 14 days, about 13 to 15 days, about 13 to 16 days, about 13 to 17 days, about 13 to 18 days, about 13 to 19 days, about 13 to 20 days, about 14 to 15 days, about 14 to 16 days, about 14 to 17 days, about 14 to 18 days, about 14 to 19 days, about 14 to 20 days, about 15 to 16 days, about 15 to 17 days, about 15 to 18 days, about 15 to 19 days, about 15 to 20 days, about 16 to 17 days, about 16 to 18 days, about 16 to 19 days, about 16 to 20 days, about 17 to 18 days, about 17 to 19 days, about 17 to 20 days, about 18 to 19 days, about 18 to 20 days, or about 19 to 20 days to prepare a population of genetically engineered NKT cells with reduced NKT cell exhaustion. In aspects, the engineered NKT cells further express a CAR. In other aspects, the engineered NKT cells express a CAR and an exogenous growth factor.

In aspects, the methods for reducing NKT cell exhaustion within an NKT cell population comprising the steps of engineering NKT cells further comprises separating desired cells from the population of genetically engineered NKT cells. In an aspect, the method further comprises separating the engineered NKT cells by the expression of CD62L to produce a selected population of CD62L(+) genetically engineered NKT cells. In an aspect, the method further comprises separating the engineered NKT cells by the expression of 4-1BB to produce a selected population of 4-1BB (+) genetically engineered NKT cells.

The present disclosure provides for, and includes, methods reducing NKT cell exhaustion within an NKT cell population comprising the steps of isolating fresh PBMCs from a donor, separating NKT cells from the PBMCs prior to culture, expanding said separated NKT cells in the presence of at least αGalCer, and a growth factor for at least 1 day to prepare NKT cells for genetic engineering. In an aspect, the growth factor comprises IL-7, IL-12, IL-15, IL-21, TNF-alpha, or a combination thereof. In an aspect, the steps of preparing NKT cells for genetic engineering further comprises separating the NKT cells from PBMCs using anti-NKT microbeads.

The methods for preparing genetically engineered NKT cells having reduced NKT cell exhaustion of the present disclosure provides for, and includes, preparing genetically engineered cell populations that can be further expanded at least two fold by challenge with antigen presenting cells. In an aspect, the antigen presenting cells are CDld+ J32 leukemia cells. In aspects, the antigen presenting cells are provided at a ratio between 10:1 and 1:10 ratio. In an aspect, the challenge with antigen presenting cells is performed at a 1:1 ratio. In aspects, the antigen presenting cells are CDTd+ J32 leukemia cells.

The methods for reducing NKT cell exhaustion of the present disclosure provides for, and includes, populations of genetically engineered NKT cells reduced NKT cell exhaustion exhibit in vivo persistence as infiltrates into neuroblastoma xenografts in humanized NSG mice.

The present disclosure provides for, and includes, methods maintaining a central memory characteristic in NKT cells comprising the steps of engineering NKT cells to express at least a protein coding sequence comprising a transcriptional activator in the Wnt signaling pathway and culturing the engineered NKT cells to prepare a population of genetically engineered NKT cells with a central memory characteristic. In an aspect, the method further provides for isolating PBMCs from a donor, separating NKT cells from the PBMCs, and expanding the separated NKT cells in the presence of at least αGalCer, IL-2, and IL-21 for at least 1 day to prepare NKT cells for genetic engineering. The cells are then transfected or transduced with the expression vectors described herein and cultured to prepare NKT cells with a central memory characteristic.

In aspects, the methods maintaining a central memory characteristic in NKT cells provide for expression of a transcriptional activator in the Wnt signaling pathway that includes, but is not limited to lymphoid enhancer binding factor 1 (LEF1, Gene ID 51176), beta-catenin ((CTNNB1, Gene ID 1499)), Smad3 (Gene ID 4088), and TLE 1 (Gene ID 7088). In an aspect, the genetically engineered NKT cell comprises a expression construct encoding LEF1 is selected from the group consisting of Reference Sequence (RefSeq) ID NOs: NP_057353.1, NP_001124185.1, and NP_001124186.1. In an aspect, the genetically engineered NKT cell expresses LEF1 and a growth factor. In an aspect, the expression construct further encodes a growth factor selected from the group consisting of IL-15, IL-2, IL-4, IL-7, and combinations thereof.

In certain aspects, the NKT cells separated by the methods according to the present disclosure are expanded in the presence of suitable stimulants (e.g., aGalCer, IL-2, and IL-21) for between 1 and 20 days as provided herein.

Also included, and provided, by the present disclosure are methods maintaining a central memory characteristic in NKT cells comprising transfecting or transducing NKT cells with an expression construct comprising (a) a chimeric antigen receptor (CAR) coding sequence comprising an ectodomain sequence, a transmembrane domain sequence, and an endodomain sequence and (b) a sequence encoding a protein sequence for a transcriptional activator in the Wnt signaling pathway to prepare a genetically engineered NKT cell.

In aspects, the methods maintaining a central memory characteristic in NKT cells provide for expression of a transcriptional activator in the Wnt signaling pathway that includes, but is not limited to lymphoid enhancer binding factor 1 (LEF1, Gene ID 51176), beta-catenin ((CTNNB1, Gene ID 1499)), Smad3 (Gene ID 4088), and TLE 1 (Gene ID 7088). In an aspect, the genetically engineered NKT cell comprises a expression construct encoding LEF1 is selected from the group consisting of Reference Sequence (RefSeq) ID NOs: NP_057353.1, NP_001124185.1, and NP_001124186.1. In an aspect, the genetically engineered NKT cell expresses LEF1 and a growth factor. In an aspect, the expression construct further encodes a growth factor selected from the group consisting of IL-15, IL-2, IL-4, IL-7, and combinations thereof.

In certain aspects, the methods maintaining a central memory characteristic in NKT cells includes genetically engineering NKT cells to comprise at least a CAR in combination with expression of a transcriptional activator in the Wnt signaling pathway. In specific aspects, NKT cells are engineered to express two or more CARs in combination with expression of a transcriptional activator in the Wnt signaling pathway. In an aspect, genetically engineering the NKT cell comprises introducing an expression construct for a transcriptional activator in the Wnt signaling pathway selected from the group consisting of lymphoid enhancer binding factor 1 (LEF1, Gene ID 51176), beta-catenin (CTNNB1), Smad3, and TLE 1. In and aspect, the transcriptional activator is LEF1 selected from the group consisting of Reference Sequence (RefSeq) ID NOs: NP_057353.1, NP_001124185.1, NP_001124186.1 in combination with one or more CARs.

Included, and provided by the methods maintaining a central memory characteristic in NKT cells are expression of CARs recognizing cancer antigens such as Melanoma-associated antigen (MAGE), Preferentially expressed antigen of melanoma (PRAME), CD19, CD20, CD22, K-light chain, CD30, CD33, CD123, CD38, CD138, ROR, ErbB2, ErbB3/4, EGFr vIII, carcinoembryonic antigen, EGP2, EGP40, HER2, mesothelin, TAG72, PSMA, NKG2D ligands, B7-H6, IL-13 receptor a2, MUC1, MUC16, CA9, GD2, GD3, HMW-MAA, CD171, Lewis Y, G250/CAIX, HLA-AI MAGE Al, HLA-A2 NY-ESO-1, PSC1, folate receptor-a, CD44v6, CD44v7/8, 8H9, NCAM, VEGF receptors, 5T4, Fetal AchR, or CD44v6. In an aspect, the cancer antigens introduced to genetically engineer NKT cells are selected from the group consisting of CD19, GD2, and GPC3. In another aspect, the antibody recognition domain of the CAR recognized the cancer antigen CD19. In an aspect, the cancer antigen recognized by the antibody recognition domain is GD2. In yet another aspect, the cancer antigen recognized by the antibody recognition domain GPC3. Methods to introduce expression constructs are known in the art. In aspects or the present disclosure the genetic engineering includes and provides for introducing the constructs of the present disclosure using retrovirus or lentivirus mediated methods.

Also included and provided for by the present disclosure methods to genetically engineer NKT cell cells to express two or more CAR molecules having antibody recognition domains that recognize two or more cancer antigens selected from the group consisting of MAGE, PRAME, CD19, CD20, CD22, K-light chain, CD30, CD33, CD123, CD38, CD138, RORl, ErbB2, ErbB3/4, EGFr viii, carcinoembryonic antigen, EGP2, EGP40, HER2, mesothelin, TAG72, PSMA, NKG2D ligands, B7-H6, IL-13 receptor a2, MUC1, MUC16, CA9, GD2, GD3, HMW-MAA, CD171, Lewis Y, G250/CAIX, HLA-AI MAGE Al, HLA-A2 NY-ESO-1, PSC1, folate receptor-a, CD44v6, CD44v7/8, 8H9, NCAM, VEGF receptors, 5T4, Fetal AchR, and CD44v6. Genetical engineering of NKT cells having two or more CAR molecules further includes constructs for expressing a Wnt signaling pathway protein and may further include sequences for expressing a growth factor selected from IL-2, IL-4, IL-7, IL-15, or IL-21. Expression constructs can be prepared separately, or more conveniently as one or more polyprotein expression constructs with CHYSEL sequences as described above. The methods of maintaining NKT cell expansion potential comprising introducing into NKT cells the expression constructs described herein, may further include isolating PMBCs from a subject, isolating NKT cells and expanding them by culture by stimulation with uGalCer-pulsed aAPCs in the presence of growth factors for between 1 and three days prior to introducing the expression constructs of the present disclosure. In certain aspects, the NKT cells separated by the methods according to the present disclosure are expanded in the presence of suitable stimulants (e.g., αGalCer, IL-2, and IL-21) for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, or at least 20 days to prepare the NKT cells for genetic engineering.

The methods maintaining a central memory characteristic in NKT cells comprising introducing into NKT cells the expression constructs described herein, may further include isolating PMBCs from a subject, isolating NKT cells and expanding them by culture in the presence of suitable stimulants (e.g., αGalCer, IL-2, and IL-21) for between 1 to 20 days, 2 to 20 days, 3 to 20 days, 4 to 20 days, 5 to 20 days, 6 to 20 days, 7 to 20 days, 8 to 20 days, 9 to 20 days, 10 to 20 days, 11 to 20 days, 12 to 20 days, 13 to 20 days, or 14 to 20 days. In certain aspects, the NKT cells separated by the methods according to the present disclosure are expanded in culture in the presence of suitable stimulants (e.g., αGalCer, IL-2, and IL-21) prior to introducing the expression constructs described herein for about 1 day, about 2 days, about 3 days, about 4 days, about 5 days, about 6 days, about 7 days, about 8 days, about 9 days, about 10 days, about 11 days, or about 12 days.

In an aspects of the methods maintaining a central memory characteristic in NKT cells, the CAR ectodomain sequences comprise an antibody recognition domain and a spacer domain.

In further aspects, resulting populations of genetically engineered NKT cells with a central memory characteristic can be expanded by culturing in the presence of antigen presenting cells, such as CD1+ J32 leukemia cells or aAPCs. In aspects, the genetically engineered NKT cells are part of a population and the number of total cells in the population are determined and cultured together with antigen presenting cells, such as CD1+ J32 leukemia cells, and are provided at a ratio between 10:1 and 1:10 ratio. In an aspect, the antigen presenting cells, such as CD1+ J32 leukemia cells can be provided at a ratio of 1:1 relative to the total number of cells in the genetically engineered NKT cell population. In an aspect, the genetically engineered NKT cells are isolated prior to challenge. In other aspects, the genetically engineered NKT cells are cultured as part of a mixed population. In an aspect the mixed population comprises at least 10% genetically engineered NKT cells.

As provided herein, the populations of genetically engineered NKT cells with a central memory characteristic retain the ability to expand at least two fold after culture with antigen presenting cells, such as CD1+ J32 leukemia cells for more than three cycles of culture wherein each cycle is performed after a period of time. In aspects, the genetically engineered NKT cells retain the ability to expand at least two fold after culture with antigen presenting cells, such as CD1+ J32 leukemia cells for between three and 20 cycles of culture. In an aspect, the genetically engineered NKT cells retain the ability to expand at least two fold after culture with antigen presenting cells, such as CD1+ J32 leukemia cells for more than four cycles of culture. In an aspect, the genetically engineered NKT cells retain the ability to expand at least two fold after culture with antigen presenting cells, such as CD1+ J32 leukemia cells for more than five cycles of culture. In an aspect, the genetically engineered NKT cells retain the ability to expand at least two fold after culture with antigen presenting cells, such as CD1+ J32 leukemia cells for more than six cycles of culture. In an aspect, the genetically engineered NKT cells retain the ability to expand at least two fold after culture with antigen presenting cells, such as CD1+ J32 leukemia cells for more than seven cycles of culture. In an aspect, the genetically engineered NKT cells retain the ability to expand at least two fold after culture with antigen presenting cells, such as CD1+ J32 leukemia cells for more than eight cycles of culture. In an aspect, the genetically engineered NKT cells retain the ability to expand at least two fold after culture with antigen presenting cells, such as CD1+ J32 leukemia cells for more than nine cycles of culture.

As provided by the present disclosure, the genetically engineered NKT cells can be expanded repeatedly after a culture period of at least one day. In aspects, the expansion is performed after a period of between one and three days. In certain aspects, the genetically engineered NKT cells are expanded for a period of at least 1 day, at least 2 days, at least 3 days, at least 4 days, at least 5 days, at least 6 days, or at least 7 days before repeating the cycle with culture with CD1+ J32 leukemia cells or other antigen presenting cells.

The present disclosure further provides for performing one or more selections on a population of cells comprising a plurality of genetically engineered NKT cells with a central memory characteristic. As provided herein, selection of genetically engineered NKT cell cells can be selection for both genetically engineered NKT cells and non-modified NKT cells and the selected population used for further culture and expansion. In aspects, the selection of genetically engineered NKT cell cells is a selection for genetically engineered NKT cells from a mixed population of cells that include both non-modified NKT cells and other cell types, usually lymphocytes. The selected populations are used for further culture and additional rounds expansion. In aspects, the selection and expansion of genetically engineered NKT cells can be performed multiple times, up to 20 times. The methods of the present disclosure thereby provide for the expansion of genetically engineered NKT cells of more than a million fold. In aspects, the methods provide for selection and expansion at least three times. The present disclosure further provides for, and includes, selection and expansion at least four times, at least five times, and at least six times. In additional aspects, the genetically engineered NKT cells are selected and expanded for at least 7 times. In further aspects, the genetically engineered NKT cells are selected and expanded for eight, nine or more times.

The present disclosure further provides for performing one or more selections on a population of cells comprising a plurality of genetically engineered NKT cells with a central memory characteristic. As provided herein, selection of genetically engineered NKT cell cells can be selection for Type I NKT cells having a central memory characteristic followed by expansion.

In aspects provided herein, selected Type I genetically engineered NKT cell populations are used for further culture and additional rounds expansion. In aspects, the selection and expansion of Type I genetically engineered NKT cells can be performed multiple times, up to 20 times. The methods of the present disclosure thereby provide for the expansion of Type I genetically engineered NKT cells of more than a million fold. In aspects, the methods provide for selection and expansion at least three times. The present disclosure further provides for, and includes, selection and expansion at least four times, at least five times, and at least six times. In additional aspects, the Type I genetically engineered NKT cells are selected and expanded for at least 7 times. In further aspects, the Type I genetically engineered NKT cells are selected and expanded for eight, nine or more times.

Also provided for, and included is the expansion of Type I genetically engineered NKT cells to produce populations that comprise at least 20% of the total population of cells. In an aspect the Type I genetically engineered NKT cells comprise at least 20% of the total population of cells after at least two rounds of expansion.

Provided for, and included in, the present disclosure are methods maintaining a central memory characteristic in NKT cells comprising the steps of engineering NKT cells to express at least a protein coding sequence comprising a transcriptional activator in the Wnt signaling pathway and culturing the engineered NKT cells to prepare a population of genetically engineered NKT cells with persistent expansion potential to produce cell populations wherein the genetically engineered NKT cells comprise greater than 10% of the total population of cells. In aspects, the 10% or greater population of genetically engineered NKT cells with persistent expansion potential comprises Type I NKT cells. In aspects, the total population comprises Type I NKT cells, Type II NKT cells, irradiated PBMC cells, non-NKT cells, and non-engineered cells. In aspects, the engineered NKT cells further express a CAR. In other aspects, the engineered NKT cells express a CAR and an exogenous growth factor.

In certain aspects, the population of genetically engineered NKT cells with a central memory characteristic within an NKT cell population according to the present disclosure comprises between at least 10% up to 80%, between 10% and 90%, between 10% and 95%, between 10% and 98%, between 10% and 99%, and up to 100% wherein non-engineered NKT cells comprise less than 99.9% of the total population. In aspects, the engineered NKT cells further express a CAR. In other aspects, the engineered NKT cells express a CAR and an exogenous growth factor.

The present disclosure provides for, and includes, methods for reducing NKT cell exhaustion comprising the steps of engineering NKT cells to express at least a protein coding sequence comprising a transcriptional activator in the Wnt signaling pathway and culturing the engineered NKT cells to prepare a population of genetically engineered NKT cells with persistent expansion potential to produce cell populations wherein the genetically engineered NKT cells comprise greater than 10% CD62L(+) NKT cells of the total population of cells. In aspects, the 10% or greater population of genetically engineered CD62L(+) NKT cells with persistent expansion potential comprises Type I NKT cells. In aspects, the total population comprises Type I NKT cells, Type II NKT cells, irradiated PBMC cells, non-NKT cells, and non-engineered cells. In aspects, the engineered CD62L(+) NKT cells further express a CAR. In other aspects, the engineered CD62L(+) NKT cells express a CAR and an exogenous growth factor.

In certain aspects, the population of genetically engineered NKT cells with a central memory characteristic within an NKT cell population according to the present disclosure comprises at least 50%, at least 55%, at least 60%, at least 65%, at least 70%, at least 75%, at least 80%, at least 85%, at least 90%, or at least 95% CD62L(+) NKT cells. In certain aspects, the population of genetically engineered NKT cells with a central memory characteristic according to the present disclosure comprises between at least 50% up to 70%, between 50% up to 80%, between 50% and 90%, between 50% and 95%, between 50% and 98%, between 50% and 99%, and up to 100% wherein non-engineered NKT cells comprise less than 99.9% of the total population In aspects, the engineered CD62L(+) NKT cells further express a CAR. In other aspects, the engineered CD62L(+) NKT cells express a CAR and an exogenous growth factor.

The present disclosure provides for, and includes, methods for maintaining a central memory characteristic in NKT cells comprising the steps of engineering NKT cells to express at least a protein coding sequence comprising a transcriptional activator in the Wnt signaling pathway and culturing the engineered NKT cells to prepare a population of genetically engineered NKT cells with a central memory characteristic, wherein said engineered NKT cells are cultured for a period of time. In an aspect, the culture period is for between 1 and 20 days. In aspects the culture period is between 1 to 2 days, 1 to 3 days, 1 to 4 days, 1 to 5 days, 1 to 6 days, 1 to 7 days, 1 to 8 days, 1 to 9 days, 1 to 10 days, 1 to 11 days, and 1 to 12 days In aspects, the engineered NKT cells further express a CAR. In other aspects, the engineered NKT cells express a CAR and an exogenous growth factor.

In aspects, the methods for maintaining a central memory characteristic in NKT cells comprising the steps of engineering NKT cells further comprises separating desired cells from the population of genetically engineered NKT cells. In an aspect, the method further comprises separating the engineered NKT cells by the expression of CD62L to produce a selected population of CD62L(+) genetically engineered NKT cells. In an aspect, the method further comprises separating the engineered NKT cells by the expression of 4-1BB to produce a selected population of 4-1BB (+) genetically engineered NKT cells.

The present disclosure provides for, and includes, methods maintaining a central memory characteristic in NKT cells comprising the steps of isolating fresh PBMCs from a donor, separating NKT cells from the PBMCs prior to culture, expanding said separated NKT cells in the presence of at least aGalCer, and a growth factor for at least 1 day to prepare NKT cells for genetic engineering. In an aspect, the growth factor comprises IL-7, IL-12, IL-15, IL-21, TNF-alpha, or a combination thereof. In an aspect, the steps of preparing NKT cells for genetic engineering further comprises separating the NKT cells from PBMCs using anti-NKT microbeads.

The methods for preparing genetically engineered NKT cells having a central memory characteristic of the present disclosure provides for, and includes, preparing genetically engineered cell populations that can be further expanded at least two fold by challenge with antigen presenting cells. In an aspect, the antigen presenting cells are CDld+ J32 leukemia cells. In aspects, the antigen presenting cells are provided at a ratio between 10:1 and 1:10 ratio. In an aspect, the challenge with antigen presenting cells is performed at a 1:1 ratio. In aspects, the antigen presenting cells are CDTd+ J32 leukemia cells.

The methods for maintaining a central memory characteristic in NKT cells of the present disclosure provides for, and includes, populations of genetically engineered NKT cells a central memory characteristic exhibit in vivo persistence as infiltrates into neuroblastoma xenografts in humanized NSG mice.

### EMBODIMENTS

1. A genetically engineered natural killer T (NKT) cell comprising an expression construct encoding a protein sequence for a transcriptional activator in the Wnt signaling pathway.

2. The genetically engineered NKT cell of embodiment 1, wherein said expression construct encodes a polyprotein comprising said protein sequence for a transcriptional activator in the Wnt signaling pathway and up to three additional protein coding sequences.

3. The genetically engineered NKT cell of embodiment 1 or 2, wherein said NKT cell is a Type I NKT cell.

4. The genetically engineered NKT cell of any one of embodiments 1 to 3, wherein said genetically engineered NKT cell is a CD62L positive (CD62L+) cell.

5. The genetically engineered NKT cell of any one of embodiments 1 to 4, wherein said genetically engineered NKT cell is positive for the expression of a marker selected from the group consisting of CD4, CD28, 4-1BB, CD45RO, CCR7, OX40, and combinations thereof.

6. The genetically engineered NKT cell of any one of embodiments 1 to 5, wherein said genetically engineered NKT cell has low levels of expression of TIM-3, LAG-3, and PD-1.

7. The genetically engineered NKT cell of any one of embodiments 1 to 6, wherein said genetically engineered NKT cell is negative for expression of CD161, CD56, CD244, TIM-3, LAG-3, TCF1, and PD-1.

8. The genetically engineered NKT cell of any one of embodiments 1 to 7, wherein said protein sequence for a transcriptional activator in the Wnt signaling pathway and up to three additional protein coding sequences are separated by an autonomous intra-ribosomal self-processing peptide.

9. The genetically engineered NKT cell of embodiment 8, wherein said autonomous intra-ribosomal self-processing is a foot-and-mouth disease virus (FMDV) 2A sequence or a related cis acting hydrolase element (CHYSEL).

10. The genetically engineered NKT cell of any one of embodiments 1 to 9, wherein said transcriptional activator is selected from the group consisting of lymphoid enhancer binding factor 1 (LEF1, Gene ID 51176), beta-catenin ((CTNNB1, Gene ID 1499)), Smad3 (Gene ID 4088), HNF1 homeobox A (HNF1A, Gene ID: 6927 (alt. TCF1), transcription factor 7 (TCF7, Gene ID:6932 (alt. TCF1) and TLE family member 1, transcriptional corepressor (TLE 1, Gene ID 7088).

11. The genetically engineered NKT cell of embodiment 10, wherein said LEF1 selected from the group consisting of Reference Sequence (RefSeq) ID NOs: NP_057353.1, NP_001124185.1, and NP_001124186.1.

12. The genetically engineered NKT cell of any one of embodiments 1 to 11, wherein said genetically engineered NKT cell further comprises an expression construct encoding a protein sequence for a chimeric antigen receptor (CAR).

13. The genetically engineered NKT cell of embodiment 12, wherein said chimeric antigen receptor (CAR) comprises an antigen recognition domain recognizing a cancer antigen.

14. The genetically engineered NKT cell of embodiment 13, wherein said cancer antigen is selected from the group consisting of CD19, GD2, and GPC3.

15. The genetically engineered NKT cell of embodiment 2, wherein said polyprotein protein sequence further comprises a protein coding sequence for a chimeric antigen receptor (CAR).

16. The genetically engineered NKT cell of embodiment 15, wherein said polyprotein protein sequence further comprises at least one protein coding sequence for a growth factor.

17. The genetically engineered NKT cell of embodiment 16, wherein said growth factor is IL-2, IL-4, IL-7, IL-15, IL-21, or combinations thereof.

18. The genetically engineered NKT cell of embodiment 16, wherein said growth factor is selected from the group consisting of interleukin-2 (IL-2), interleukin-21 (IL-21), and interleukin-15 (IL-15).

19. A population of cells comprising a plurality of genetically engineered NKT cells comprising expression construct encoding a protein sequence for a transcriptional activator in the Wnt signaling pathway.

20. The population of cells of embodiment 19, wherein said population further comprises cells including Type I NKT cells, Type II NKT cells, irradiated PBMC cells, non-NKT cells, or non-engineered cells.

21. The population of cells of embodiment 19 or 20, wherein said plurality of genetically engineered NKT cells is greater than 10% of the total cell population.

22. The population of cells of embodiment 21, wherein said plurality of genetically engineered NKT cells is 25% to 30% of the total cell population.

23. The population of cells of any one of embodiments 19 to 22, wherein said plurality of genetically engineered NKT cells comprises at least 50% CD62L(+) NKT cells.

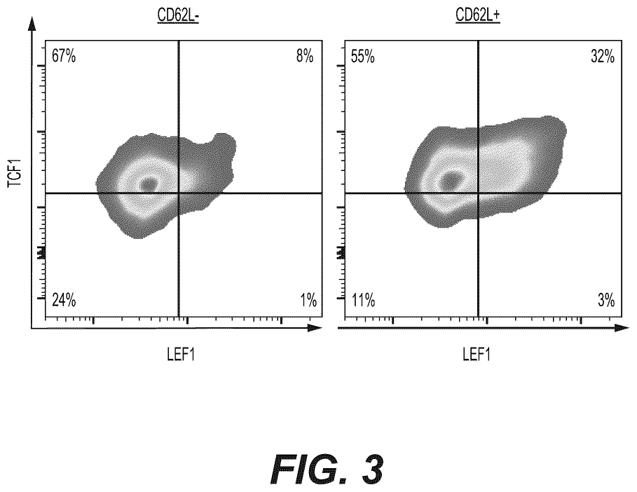

24. The population of cells of any one of embodiments 19 to 23, wherein at least 30 percent of said plurality of genetically engineered NKT cells express LEF1. [FIG. 3b]

25. A therapeutically effective amount of the population of cells of any one of embodiments 19 to 24.

26. A chimeric antigen receptor expression construct comprising (a) a chimeric antigen receptor (CAR) coding sequence comprising an ectodomain sequence, a transmembrane domain sequence, and an endodomain sequence, and (b) a sequence encoding a protein sequence for a transcriptional activator in the Wnt signaling pathway.

27. The chimeric antigen receptor expression construct of embodiment 26, wherein said transcriptional activator in the Wnt signaling pathway comprises lymphoid enhancer binding factor 1 (LEF1, Gene ID 51176), beta-catenin ((CTNNB1, Gene ID 1499)), Smad3 (Gene ID 4088), HNF1 homeobox A (HNF1A, Gene ID: 6927 (alt. TCF1), transcription factor 7 (TCF7, Gene ID:6932 (alt. TCF1) or TLE family member 1, or transcriptional corepressor (TLE 1, Gene ID 7088).

28. The chimeric antigen receptor expression construct of embodiment 26, wherein said transcriptional activator in the Wnt signaling pathway is LEF1.

29. The chimeric antigen receptor expression construct of any one of embodiments 26 to 28, wherein said CAR coding sequences and said transcriptional activator sequence are in frame, and wherein said expression construct further comprises a foot-and-mouth disease virus (FMDV) 2A sequence or a FMDV 2A related cis acting hydrolase element (CHYSEL) sequence between said CAR and said LEF1 coding sequences.

30. The chimeric antigen receptor expression construct of embodiment 28, wherein said CAR coding sequences and said LEF1 coding sequence are in frame, and wherein said expression construct further comprises a foot-and-mouth disease virus (FMDV) 2A sequence or a FMDV 2A related cis acting hydrolase element (CHYSEL) sequence between said CAR and said LEF1 coding sequences.

31. The chimeric antigen receptor expression construct of any one of embodiments 26 to 30, further comprising an in-frame interleukin-15 (IL-15) coding sequence separated by a foot-and-mouth disease virus (FMDV) 2A sequence or a FMDV 2A related cis acting hydrolase element (CHYSEL) sequence.

32. The chimeric antigen receptor expression construct of embodiment 31, further comprising a second foot-and-mouth disease virus (FMDV) 2A sequence or a FMDV 2A related cis acting hydrolase element (CHYSEL) sequence.

33. The chimeric antigen receptor expression construct of any one of embodiments 26 to 32, wherein said ectodomain sequences comprises an antigen recognition domain and a spacer domain.

34. The chimeric antigen receptor expression construct of embodiment 33, wherein said antigen recognition domain binds to an antigen selected from CD19, GD2, or GPC3.

35. The chimeric antigen receptor expression construct of any one of embodiments 26 to 34, further comprising an in-frame coding sequence for an additional protein separated by a foot-and-mouth disease virus (FMDV) 2A sequence or a FMDV 2A related cis acting hydrolase element (CHYSEL) sequence.

36. The chimeric antigen receptor expression construct of embodiment 34, wherein said antigen recognition domain comprises a single-chain variable fragment (scFv) from the CD19-specific antibody FMC-63.

37. The chimeric antigen receptor expression construct of embodiment 34, wherein said antigen recognition domain comprises a single-chain variable fragment (scFv) from the GD2-specific antibody 14G2a.

38. The chimeric antigen receptor expression construct of any one of embodiments 26 to 37, wherein said endodomain comprises the signal sequence of 4-1BB fused in-frame to a CD3-zeta chain.

39. A genetically engineered NKT cell comprising a chimeric antigen receptor expression construct of any one of embodiments 26 to 38.

40. A method of maintaining NKT cell expansion potential comprising the steps of engineering NKT cells to express at least a protein coding sequence comprising a transcriptional activator in the Wnt signaling pathway; and culturing said engineered NKT cells to prepare a population of genetically engineered NKT cells with persistent expansion potential.

41. A method of maintaining NKT cell expansion potential of embodiment 40, wherein said engineering comprises transfecting or transducing said NKT cells with an expression construct comprising (a) a chimeric antigen receptor (CAR) coding sequence comprising an ectodomain sequence, a transmembrane domain sequence, and an endodomain sequence and (b) a sequence encoding a protein sequence for a transcriptional activator in the Wnt signaling pathway.

42. The method of embodiment 41, wherein said ectodomain sequences comprises an antigen recognition domain and a spacer domain.

43. The method of embodiment 42, wherein said antigen recognition domain binds to an antigen selected from CD19, GD2, or GPC3.

44. The method of any one of embodiments 40 to 43, wherein said engineering comprises transducing said protein expression construct using a retrovirus or lentivirus.

45. The method of any one of embodiments 40 to 44, wherein said transcriptional activator in the Wnt signaling pathway comprises lymphoid enhancer binding factor 1 (LEF1, Gene ID 51176), beta-catenin ((CTNNB1, Gene ID 1499)), Smad3 (Gene ID 4088), HNF1 homeobox A (HNF1A, Gene ID: 6927 (alt. TCF1), transcription factor 7 (TCF7, Gene ID:6932 (alt. TCF1) or TLE family member 1, transcriptional corepressor (TLE 1, Gene ID 7088).

46. The method of embodiment 41, wherein said transcriptional activator of the Wnt signaling pathway is LEF1 selected from the group consisting of Reference Sequence (RefSeq) ID NOs: NP_057353.1, NP_001124185.1, and NP_001124186.1.

47. The method of any one of embodiments 40 to 46, further comprising expanding said population of genetically engineered NKT cells with persistent expansion potential expand at least two fold by first challenge with CD1+ J32 leukemia cells at a 1:1 ratio.

48. The method of embodiment 47, wherein said expanding is repeated by one or more further challenges with CD1+ J32 leukemia cells at a 1:1 ratio after a period of time.

49. The method of embodiment 48, wherein said period is every one to three days.

50. The method of embodiment 48, wherein said expanding is repeated 2 to 9 more times, and said population of genetically engineered NKT cells are a population of NKT cells with persistent expansion potential.

51. The method of any one of embodiments 40 to 50, further comprising performing a selection on said population of genetically engineered NKT cells to prepare a separated population of genetically engineered NKT cells.

52. The method of embodiment 51, wherein said separated population of genetically engineered NKT cells are Type I NKT cells.

53. The method of embodiment 52, wherein said Type I NKT cells comprise at least 20% of the total population of cells.

54. The method of any one of embodiments 40 to 53, wherein said population of genetically engineered NKT cells with persistent expansion potential comprise greater than 10% of the total population of cells.

55. The method of any one of embodiments 40 to 54, wherein said population of genetically engineered NKT cells with persistent expansion potential comprises Type I NKT cells, Type II NKT cells, irradiated PBMC cells, non-NKT cells, and non-engineered cells.

56. The method of any one of embodiments 40 to 55, wherein said population of genetically engineered NKT cells with persistent expansion potential comprises at greater than 50% of the total cell population.

57. The method of any one of embodiments 40 to 56, wherein said population of genetically engineered NKT cells with persistent expansion potential comprises at least 10% CD62L(+) NKT cells.

58. The method of any one of embodiments 40 to 57, further comprising separating said engineered NKT cells by the expression of CD62L.

59. The method of any one of embodiments 40 to 58, further comprising separating said engineered NKT cells by the expression of 4-1BB.

60. The method of any one of embodiments 40 to 59, wherein said population of genetically engineered NKT cells with persistent expansion potential exhibit in vivo persistence as infiltrates into neuroblastoma xenografts in humanized NSG mice.

61. The method of maintaining NKT cell expansion potential of any one of embodiments 40 to 60, further comprising the steps of.
a. isolating PBMCs from a donor;
b. separating NKT cells from said PBMCs; and
c. expanding said separated NKT cells in the presence of at least aGalCer, IL-2, and IL-21 for at least 1 day to prepare NKT cells for genetic engineering.

62. The method of embodiment 61, wherein said expanded population is obtained at 12 to 14 days of culture.

63. The method of any one of embodiments 61 and 62, wherein said expanded population of genetically engineered NKT cells can be further expanded at least two fold by challenge with CD1d+ J32 leukemia cells at a 1:1 ratio.

64. The method of any one of embodiments 61 to 63, wherein expanding said separated NKT cells in media further comprising IL-7, IL-15, IL-12, TNF-alpha, or a combination thereof.

65. The method of any one of embodiments 61 to 64, wherein said NKT cells are separated from said PBMCs using anti-NKT microbeads.

66. The method of any one of embodiments 61 to 65, wherein said cells are expanded for 3 days before engineering.

67. The method of any one of embodiments 61 to 66, wherein said NKT cells are expanded for to 20 days.

68. The method of any one of embodiments 61 to 67, wherein said total culturing time of said separated NKT cells and said engineered NKT cells is less than 20 days.

69. A method of reducing NKT cell exhaustion in an NKT cell population comprising the steps of
engineering NKT cells to express at least a protein coding sequence comprising a transcriptional activator in the Wnt signaling pathway; and
culturing said engineered NKT cells to prepare a population of genetically engineered NKT cells with reduced NKT cell exhaustion.

70. The method of reducing NKT cell exhaustion in an NKT cell population of embodiment 69, wherein said engineering comprises transfecting or transducing said NKT cells with an expression construct comprising (a) a chimeric antigen receptor (CAR) coding sequence comprising an ectodomain sequence, a transmembrane domain sequence, and an endodomain sequence and (b) a sequence encoding a protein sequence for a transcriptional activator in the Wnt signaling pathway.

71. The method of reducing NKT cell exhaustion in an NKT cell population of embodiment 70, wherein said ectodomain sequences comprises an antigen recognition domain and a spacer domain.

72. The method of reducing NKT cell exhaustion in an NKT cell population of embodiment 71, wherein said antigen recognition domain binds to an antigen selected from CD19, GD2, or GPC3.

73. The method of reducing NKT cell exhaustion in an NKT cell population of any one of embodiments 69 to 72, wherein said engineering comprises transducing said protein expression construct using a retrovirus or lentivirus.

74. The method of reducing NKT cell exhaustion in an NKT cell population of any one of embodiments 69 to 73, wherein said transcriptional activator in the Wnt signaling pathway comprises lymphoid enhancer binding factor 1 (LEF1, Gene ID 51176), beta-catenin ((CTNNB1, Gene ID 1499)), Smad3 (Gene ID 4088), HNF1 homeobox A (HNF1A, Gene ID: 6927 (alt. TCF1), transcription factor 7 (TCF7, Gene ID:6932 (alt. TCF1) or TLE family member 1, transcriptional corepressor (TLE 1, Gene ID 7088).

75. The method of reducing NKT cell exhaustion in an NKT cell population of any one of embodiments 69 to 74, wherein said transcriptional activator of the Wnt signaling pathway is LEF1 selected from the group consisting of Reference Sequence (RefSeq) ID NOs: NP_057353.1, NP_001124185.1, and NP_001124186.1.

76. The method of reducing NKT cell exhaustion in an NKT cell population of any one of embodiments 69 to 75, further comprising expanding said population of genetically engineered NKT cells with persistent expansion potential expand at least two fold by first challenge with CD1+ J32 leukemia cells at a 1:1 ratio.

77. The method of reducing NKT cell exhaustion in an NKT cell population of embodiment 76, wherein said expanding is repeated by one or more further challenges with CD1+ J32 leukemia cells at a 1:1 ratio after a period of time.

78. The method of reducing NKT cell exhaustion in an NKT cell population of embodiment 77, wherein said period is every one to three days.

79. The method of reducing NKT cell exhaustion in an NKT cell population of embodiment 77, wherein said expanding is repeated 2 to 9 more times, and said population of genetically engineered NKT cells are a population of NKT cells with persistent expansion potential.

80. The method of reducing NKT cell exhaustion in an NKT cell population of any one of embodiments 69 to 79, further comprising performing a selection on said population of genetically engineered NKT cells to prepare a separated population of genetically engineered NKT cells.

81. The method of reducing NKT cell exhaustion in an NKT cell population of embodiment 80, wherein said separated population of genetically engineered NKT cells are Type I NKT cells.

82. The method of reducing NKT cell exhaustion in an NKT cell population of embodiment 81, wherein said Type I NKT cells comprise at least 20% of the total population of cells.

83. The method of reducing NKT cell exhaustion in an NKT cell population of any one of embodiments 69 to 82, wherein said population of genetically engineered NKT cells with persistent expansion potential comprise greater than 10% of the total population of cells.

84. The method of reducing NKT cell exhaustion in an NKT cell population of any one of embodiments 69 to 83, wherein said population of genetically engineered NKT cells with persistent expansion potential comprises Type I NKT cells, Type II NKT cells, irradiated PBMC cells, non-NKT cells, and non-engineered cells.

85. The method of reducing NKT cell exhaustion in an NKT cell population of any one of embodiments 69 to 84, wherein said population of genetically engineered NKT cells with persistent expansion potential comprises at greater than 50% of the total cell population.

86. The method of reducing NKT cell exhaustion in an NKT cell population of any one of embodiments 69 to 85, wherein said population of genetically engineered NKT cells with persistent expansion potential comprises at least 10% CD62L(+) NKT cells.

87. The method of reducing NKT cell exhaustion in an NKT cell population of any one of embodiments 69 to 86, further comprising separating said engineered NKT cells by the expression of CD62L.

88. The method of reducing NKT cell exhaustion in an NKT cell population of any one of embodiments 69 to 87, further comprising separating said engineered NKT cells by the expression of 4-1BB.

89. The method of any one of embodiments 69 to 88, further comprising the steps of
a. isolating PBMCs from a donor;
b. separating NKT cells from said PBMCs; and
c. expanding said separated NKT cells in the presence of at least aGalCer, IL-2, and IL-21 for at least 1 day to prepare NKT cells for genetic engineering.

90. The method of reducing NKT cell exhaustion in an NKT cell population of embodiment 89, wherein said expanded population is obtained at 12 to 14 days of culture.

91. The method of reducing NKT cell exhaustion in an NKT cell population of embodiment 89 or 90, wherein said NKT cells are separated from said PBMCs using anti-NKT microbeads.

92. The method of reducing NKT cell exhaustion in an NKT cell population of any one of embodiments 89 to 91, wherein expanding said separated NKT cells in media further comprising IL-7, IL-15, IL-12, IL-21, TNF-alpha, or a combination thereof.

93. The method of reducing NKT cell exhaustion in an NKT cell population of any one of embodiments 89 to 92, wherein said cells are expanded for 3 days before engineering.

94. The method of reducing NKT cell exhaustion in an NKT cell population of any one of embodiments 89 to 93, wherein said NKT cells are expanded for 10 to 20 days.

95. The method of reducing NKT cell exhaustion in an NKT cell population of embodiment 94, wherein said total culturing time of said separated NKT cells and said engineered NKT cells is less than 20 days.

96. A method of maintaining a central memory characteristic in NKT cells, comprising the steps of:
engineering an NKT cell with an expression construct to express a sequence encoding a protein that is a transcriptional activator of the Wnt signaling pathway;
culturing said engineered NKT cell to express said transcriptional activator; and
preparing a population of genetically engineered NKT cells that retain proliferative ability and cytotoxicity of the central memory NKT cell characteristic after repeated challenge.

97. The method of maintaining a central memory characteristic in NKT cells of embodiment 96, wherein said expression construct further comprises a sequence encoding a chimeric antigen receptor (CAR), said CAR comprising an ectodomain sequence, a transmembrane domain sequence, and an endodomain sequence.

98. The method of maintaining a central memory characteristic in NKT cells of embodiment 97, wherein said ectodomain sequences comprises an antigen recognition domain and a spacer domain.

99. The method of maintaining a central memory characteristic in NKT cells of embodiment 98, wherein said antigen recognition domain binds to an antigen selected from CD19, GD2, or GPC3.

100. The method of reducing NKT cell exhaustion in an NKT cell population of any one of embodiments 96 to 99, wherein said engineering comprises transducing said protein expression construct using a retrovirus or lentivirus.

101. The method of maintaining a central memory characteristic in NKT cells of any one of embodiments 96 to 100, wherein said transcriptional activator in the Wnt signaling pathway comprises lymphoid enhancer binding factor 1 (LEF1, Gene ID 51176), beta-catenin ((CTNNB1, Gene ID 1499)), Smad3 (Gene ID 4088), HNF1 homeobox A (HNF1A, Gene ID: 6927 (alt. TCF1), transcription factor 7 (TCF7, Gene ID:6932 (alt. TCF1) or TLE family member 1, transcriptional corepressor (TLE 1, Gene ID 7088).

102. The method of maintaining a central memory characteristic in NKT cells of embodiment 101, wherein said transcriptional activator of the Wnt signaling pathway is LEF1 selected from the group consisting of Reference Sequence (RefSeq) ID NOs: NP_057353.1, NP_001124185.1, and NP_001124186.1.

103. The method of maintaining a central memory characteristic in NKT cells of any one of embodiments 96 to 102, further comprising expanding said population of genetically engineered NKT cells with persistent expansion potential expand at least two fold by first challenge with CD1+ J32 leukemia cells at a 1:1 ratio.

104. The method of maintaining a central memory characteristic in NKT cells of embodiment 103, wherein said expanding is repeated by one or more further challenges with CD1+ J32 leukemia cells at a 1:1 ratio after a period of time.

105. The method of maintaining a central memory characteristic in NKT cells of embodiment 104, wherein said period is every one to three days.

106. The method of maintaining a central memory characteristic in NKT cells of embodiment 105, wherein said expanding is repeated 2 to 9 more times, and said population of genetically engineered NKT cells are a population of NKT cells with persistent expansion potential.

107. The method of maintaining a central memory characteristic in NKT cells of any one of embodiments 96 to 106, further comprising performing a selection on said population of genetically engineered NKT cells to prepare a separated population of genetically engineered NKT cells.

108. The method of maintaining a central memory characteristic in NKT cells of embodiment 107, wherein said separated population of genetically engineered NKT cells are Type I NKT cells.

109. The method of maintaining a central memory characteristic in NKT cells of embodiment 108, wherein said Type I NKT cells comprise at least 20% of the total population of cells.

110. The method of maintaining a central memory characteristic in NKT cells of any one of embodiments 96 to 109, wherein said population of genetically engineered NKT cells with persistent expansion potential comprise greater than 10% of the total population of cells.

111. The method of maintaining a central memory characteristic in NKT cells of any one of embodiments 96 to 110, wherein said population of genetically engineered NKT cells with persistent expansion potential comprises Type I NKT cells, Type II NKT cells, irradiated PBMC cells, non-NKT cells, and non-engineered cells.

112. The method of maintaining a central memory characteristic in NKT cells of any one of embodiments 96 to 111, wherein said population of genetically engineered NKT cells with persistent expansion potential comprises at greater than 50% of the total cell population.

113. The method of maintaining a central memory characteristic in NKT cells of any one of embodiments 96 to 112, wherein said population of genetically engineered NKT cells with persistent expansion potential comprises at least 10% CD62L(+) NKT cells.

114. The method of maintaining a central memory characteristic in NKT cells of any one of embodiments 96 to 113, further comprising separating said engineered NKT cells by the expression of CD62L.

115. The method of maintaining a central memory characteristic in NKT cells of any one of embodiments 96 to 114, further comprising separating said engineered NKT cells by the expression of 4-1BB.

116. The method of maintaining a central memory characteristic in NKT cells of any one of embodiments 96 to 115, wherein said engineering comprises transfecting or transducing said NKT cells with an expression construct comprising (a) a chimeric antigen receptor (CAR) coding sequence comprising an ectodomain sequence, a transmembrane domain sequence, and an endodomain sequence and (b) a sequence encoding a protein sequence for a transcriptional activator in the Wnt signaling pathway.

117. The method of maintaining a central memory characteristic in NKT cells of any one of embodiments 96 to 116, further comprising the steps of.
a. isolating PBMCs from a donor;
b. separating NKT cells from said PBMCs; and
c. expanding said separated NKT cells in the presence of at least αGalCer, IL-2, and IL-21 for at least 1 day to prepare NKT cells for genetic engineering.

118. The method of maintaining a central memory characteristic in NKT cells of embodiment 117, wherein said expanded population is obtained at 12 to 14 days of culture.

119. The method of maintaining a central memory characteristic in NKT cells of embodiment 117 or 118, wherein expanding said separated NKT cells in media further comprising IL-7, IL-15, IL-12, IL-21, TNF-alpha, or a combination thereof.

120. The method of maintaining a central memory characteristic in NKT cells of any one of embodiments 117 to 119, wherein said cells are expanded for 3 days before engineering.

121. The method of maintaining a central memory characteristic in NKT cells of any one of embodiments 117 to 120, wherein said NKT cells are expanded for 10 to 20 days.

122. The method of maintaining a central memory characteristic in NKT cells of any one of embodiments 117 to 121, wherein said total culturing time of said separated NKT cells and said engineered NKT cells is less than 20 days.

123. The method of maintaining a central memory characteristic in NKT cells of any one of embodiments 117 to 122, wherein said NKT cells are separated from said PBMCs using anti-NKT microbeads.

While the present disclosure has been described with reference to certain embodiments, it will be understood by those skilled in the art that various changes may be made and equivalents may be substituted for elements thereof to adapt to particular situations without departing from the scope of the present disclosure. Therefore, it is intended that the present disclosure not be limited to the particular embodiments disclosed as the best mode contemplated for carrying out the present disclosure, but that the present disclosure will include all embodiments falling within the scope and spirit of the appended claims.

Any references cited herein are incorporated by reference in their entireties.

### EXAMPLES

### Example 1: Materials and Methods

### Cell Lines

K562, Daudi, Raji, Ramos, and 293T cells are purchased from the American Type Culture Collection (Manassas, VA). Cell lines CDld+ J32 leukemia cells are purchased from the American Type Culture Collection (Manassas, VA). All lines are cultured in RPMI (HyClone, Logan, UT). Media are supplemented with 10% FBS (Life Technologies, Carlsbad, CA) and 2 mM GlutaMAX™-I (Gibco, Waltham, MA). Discarded cord blood NKT cells are obtained from the MD Anderson Cancer Center Cord Blood Bank and used according to the protocols approved by the Institutional Review Boards at MD Anderson Cancer Center and Baylor College of Medicine.

The B-8-2 clone of the K562 cell line was derived previously in our laboratory (Tian et al., “CD62L+ NKT cells have prolonged persistence and antitumor activity in vivo,” J Clin Invest 126, 2341-2355 (2016).). CHLA-255 and Jurkat J32 cell lines are as previously described (Seeger et al., “Morphology, growth, chromosomal pattern and fibrinolytic activity of two new human neuroblastoma cell lines,” Cancer Res 37, 1364-1371 (1977) and Makni et al., “Reconstitution of an active surface CD2 by DNA transfer in CD2-CD3+ Jurkat cells facilitates CD3-T cell receptor-mediated IL-2 production,” J Immunol 146, 2522-2529 (1991)). CHLA-255 and 293T were cultured in IMDM (HyClone) and all other lines were maintained in RPMI 1640 (HyClone). Cell culture medium was supplemented with 2 mM GlutaMAX-I (Life Technologies) and 10% FBS (HyClone) with the exception of CHLA-255 medium, which was cultured with 20% FBS.

### Retroviral Constructs and Retrovirus Production.

CAR.CD19 and CAR.GD2 constructs are made as previously described (Heczey et al., (2014); Pule et al., 2005; and Xu et al., “NKT Cells Coexpressing a GD2-Specific Chimeric Antigen Receptor and IL15 Show Enhanced In Vivo Persistence and Antitumor Activity against Neuroblastoma,” Clin Cancer Res 25, 7126-7138 (2019)) and contained a scFv from the CD19-specific antibody FMC-63 or the GD2-specific antibody 14G2a connected via a short spacer derived from the IgG1 hinge region to the transmembrane domain derived from CD8a, followed by signaling endodomain sequences of 4-1BB fused with the CD3(signaling chain. Additional constructs are shown in FIG. 27 that further include the indicated genes linked together to a sequence encoding the human LEF1 long isoform using 2A sequence peptides derived from foot-and-mouth disease virus. FIG. 27 a-d present CAR.GD2 LEF1 constructs.

Retroviral supernatants are produced by transfection of 293T cells with a combination of chimeric antigen containing plasmids, RDF plasmid encoding the RD 114 envelope and PegPam3 plasmid encoding the MoMLV gag-pol as previously described (Vera et al., “T lymphocytes redirected against the kappa light chain of human immunoglobulin efficiently kill mature B lymphocyte-derived malignant cells,” Blood 108(12):3890-3897 (2006)).

As indicated, a CAR.GD2 construct includes LEF1 downstream of CD3ζ separated by a p2A sequence. Non-CAR retroviral constructs include GFP.firefly luciferase (GFP.FFLuc) and GFP.LEF1, which consists of GFP as a transduction marker separated from the over-expressed target protein by a p2A sequence. The lentiviral construct encoding the 7-TGC Wnt reporter construct is a generous gift from Dr. J. Rosen and Dr. K. Roarty (Baylor College of Medicine [BCM]) and the envelope plasmid pMD2.G, and packaging plasmid 68.2 are generous gifts from Dr. K. Scott and Dr. Y.-H. Tsang (BCM). Lentiviral supernatants are generated from 293T cells transfected with the relevant lentiviral construct, pMD2.G, and 68.2. To enhance transduction efficiency, lentiviral supernatants are concentrated using Retro-X Concentrator (Takara Bio) according to the manufacturer's protocol.

### NKT-Cell Isolation, Transduction, Expansion and Sorting.

PBMC of healthy donors (at least 18 years old) are isolated by gradient centrifugation from buffy coats purchased from Gulf Coast Regional Blood Center. NKTs are purified by anti-iNKT microbeads (Miltenyi Biotec). The negative PBMC fraction is irradiated (40 Gy) and aliquoted. NKTs are stimulated with an aliquot of autologous PBMCs pulsed with 100 ng/niL αGalCer (Kyowa Hakko Kirin). Recombinant IL-2 (200 U/ml, National Cancer Institute Frederick) is added every other day in complete RPMI (HyClone RPMI 1640, 10% heat inactivated fetal bovine serum and 2 mM Glutamax). NKTs are expanded for 10 days and then re-stimulated with autologous PBMC (irradiated with 40 Gy) or Ramos cells as aAPC (irradiated with 100 Gy) when indicated. On day 3 after re-stimulation, 24 well, non-tissue culture plates are coated with retronectin (Takara Bio) and after washing inoculated with 1 ml of retroviral supernatant containing the constructs shown in FIG. 27 and described above and spun for 60 min at 4600 G. The viral supernatant is then removed and stimulated NKTs are added to the wells in complete media and 200 U/ml rhIL-2. Cells are removed from the plate after 48 h, washed, re-suspended at the concentration 10⁶ cell/ml in complete RPMI with 200 U/ml IL-2 and plated for continued expansion. NKT-cell number is determined by Trypan Blue (Life technologies) counting. When indicated, NKTs or CAR-NKTs are labeled with CD62L-PE mAb (GREG-56, BD Biosciences) and anti-PE microbeads (Miltenyi) followed by magnetic sorting into CD62L+ and CD62L− subsets according to the manufacturer's instructions. The phenotype of the sorted cells was determined by FACS.

### Proliferation and Apoptosis Assays

NKTs are labeled with CellTrace Violet (CTV; Thermo Fisher, Waltham, MA) and stimulated with uGalCer-pulsed B-8-2 cells. Cell proliferation is examined on day 6 by measuring CTV dilution using flow cytometry. Early and late apoptosis is measured on day 3 post-NKT stimulation by staining for annexin-V and 7-AAD (BD Biosciences, Franklin Lakes, NJ), respectively, followed by flow cytometry.

Multiplex cytokine quantification assay CD19-CAR-NKTs are stimulated for 24 hours by Daudi lymphoma cells or CHLA-255 neuroblastoma cells at a 1:1 effector to target (E:T) ratio. Supernatants are collected and analyzed using the MILLIPLEX MAP Human Cytokine/Chemokine Immunoassay panel (Millipore) for Luminex® analysis according to the manufacturer's protocol.

Flow cytometry.

Immunophenotyping is performed using the following mAbs to: HLA-C EMR8-5, CD Id CDld42, CD86 2331, 4-1BBL C65-485, OX40L ik-1, CD3 OKT, Va24-Jal8 6B11, CD4 SK3, CD62L DREG-56, CD134 ACT35, CD137 4B4-1, PD-1 EH12.1, GATA3 L50-823 (BD Biosciences), LAG-3 Polyclonal, TEVI-3 344823 (R&D System), and rabbit anti-LEF1 EP2030Y mAb (ABCAM). BD or R&D-suggested fluorochrome and isotype-matching Abs is used as negative controls. The expression of CAR.CD19 on NKTs is determined using anti-Id (clone 136.20.1) CD19-CAR specific mAb (Torikai H, et al. Toward eliminating HLA class I expression to generate universal cells from allogeneic donors. fi/oo<i.2013; 122(8):1341-1349) and goat anti-mouse IgG (BD Biosciences).

### NKT-Cell Phenotypic Analysis

NKT-cell phenotype is assessed using monoclonal antibodies (mAbs) for CD3 (UCHT1), Vα24-Jα18 (6B11), CD4 (RPA-T4), granzyme B (GB11), CD62L (DREG-56; BD Biosciences, San Jose, CA), VO 11 (C21; Beckman Coulter, Brea, CA), and IL-21R (17A12; BioLegend, San Diego, CA and BD Biosciences). CD19-CAR expression by transduced NKTs is detected using anti-Id mAb (14G2a anti-idiotype 1A7 mAb, clone 136.20.1) (25), a gift from Dr. B. Jena (MD Anderson Cancer Center, Houston, TX). Intracellular staining is performed using a fixation/permeabilization solution kit (BD Biosciences) with mAbs for Bc12 (N46-467; BD Biosciences) and BIM (Y36; Abcam, Cambridge, MA) followed by staining with a secondary goat anti-rabbit IgG-AF488 mAb (Abcam). Phosflow staining is performed using Cytofix buffer (BD Biosciences) and Perm buffer III (BD Biosciences) with mAb for Stat3 (pY705; Clone 4; BD Biosciences). Detection of Stat3 phosphorylation is performed after 15 minutes of treatment with IL-21. Fluorochrome- and isotype-matching antibodies suggested by BD Biosciences or R&D Systems is used as negative controls.

Mitochondrial mass is measured using 25 nM MitoTracker Deep Red or MitoTracker Green (ThermoFisher Scientific) according to the manufacturer's protocol. Transcription factor staining is performed using the eBioscience Foxp3/Transcription Factor Staining Buffer Set (ThermoFisher Scientific) with mAbs for long isoforms of LEF1 (C12A5) and TCF1 (C63D9; Cell Signaling Technology) and all isoforms of LEF1 (EP2030Y, Abcam). Fluorochrome- and isotype-matching mAbs suggested by BD Biosciences, Cell Signaling Technology, or R&D Systems are used as negative controls. Analysis is performed on an LSR II five-laser flow cytometer (BD Biosciences) using BD FACSDiva software version 6.0 and FlowJo 10.7 (Tree Star).

Analysis is performed on an LSR-II 5-laser flow cytometer (BD Biosciences) using BD FACSDiva software version 6.0 and FlowJo 10.1 (Tree Star, Ashland, OR).

### In Vitro Cytotoxicity Assay

Cytotoxicity of parental and CD19-CAR-NKTs against Ramos and Raji cells, respectively, is evaluated using a 4-hour luciferase assay as previously described (13). CAR-mediated cytotoxicity of CAR.GD2-NKTs against CHLA-255 cells is evaluated using a 4 h luciferase assay as previously described (Heczey et al., “Invariant NKT cells with chimeric antigen receptor provide a novel platform for safe and effective cancer immunotherapy,” Blood 124, 2824-2833 (2014)).

### In Vitro Repeated Killing Assay

J32 leukemia cells are pulsed with uGalCer (100 ng/mL) overnight followed by CellTrace Violet (ThermoFisher Scientific) staining. Labelled J32 cells are co-cultured with NKTs transduced with GFP.LEF1 or GFP.FFLuc at the indicated E:T ratio in the presence of IL-2 (50 U/mL). On day 3, tumor killing and NKT cell expansion are measured using CountBright Absolute Counting Beads (ThermoFisher Scientific) by flow cytometry. Fresh αGalCer-pulsed J32 cells are added to the culture according to NKT cell number to re-establish the E:T ratio. This step is repeated every 3 days of co-culture. NKT cell exhaustion/memory phenotype is assessed by flow cytometry after 6 days of resting from J32 stimulation using anti-PD-1, anti-TIM-3 and anti-CD62L mAbs.

### Seahorse Assays

The extracellular acidification rate (ECAR) and oxygen consumption rate (OCR) are measured using the Agilent Seahorse XFe96 Analyzer (Agilent) per the manufacturer's instructions. GFP.FFLuc-NKTs and GFP.LEF1-NKTs are assayed on day 12 post-secondary stimulation after normalization of transduction rate.

### Gene Expression Analysis

Total RNA is collected using the Direct-zol™ RNA MiniPrep Kit (Zymo Research, Irvine, CA). Gene expression analysis is performed using the Immunology Panel version 2 (NanoString, Seattle, WA) with the nCounter Analysis System by the BCM Genomic and RNA Profiling Core. Data is analyzed using nSolver 3.0 software (NanoString). Differences in gene expression levels between CD62L+ and CD62L− subsets in the two culture conditions are evaluated using the paired moderated t-statistic of the Linear Models for Microarray Data (Limma) analysis package (26).

### Real-Time Quantitative PCR

Wnt signaling components and downstream gene expression are measured in NKTs by qPCR with specific primers. Briefly, RNA is extracted from unsorted or CD62L+/−sorted NKTs using the RNeasy Plus Mini Kit (QIAGEN). Isolated RNA is then subjected to reverse transcription and qPCR reaction using the Power SYBR Green RNA-to-CT 1-Step Kit (ThermoFisher Scientific). Relative expression of Wnt target genes in CD62L+ versus CD62L-NKTs is determined using the ΔΔCt method with GAPDH as housekeeping gene. To detect expression of Wnt receptors, co-receptors, and ligands on NKTs and K562 artificial antigen presenting cells (aAPCs), Ct values of target gene expression are compared to the Ct values of positive (GAPDH) and negative (CD19) controls. Ct values within the range of GAPDH and CD19 are considered significantly expressed. Primers are listed in Table 2.

TABLE 2
List of primers used in real-time quantitative PCR

| SEQ ID NO | Genes | Sequences |
|---|---|---|
| SEQ ID NO: 1 | GAPDH F | 5′-GAAGGTGAAGGTCGGAGTC-3′ |
| SEQ ID NO: 2 | GAPDH R | 5′-GAAGATGGTGATGGGATTTC-3′ |
| SEQ ID NO: 3 | CD19 F | 5′-TGCCCCGTCTTATGGAAACC-3′ |
| SEQ ID NO: 4 | CD19 R | 5′-CTCTTCTTCTGGGCCCACTC-3′ |
| SEQ ID NO: 5 | AXIN 2 F | 5′-ACTGCCCACACGATAAGGAG-3′ |
| SEQ ID NO: 6 | AXIN 2 R | 5′-CTGGCTATGTCTTTGCACCA-3′ |
| SEQ ID NO: 7 | TCF1 F | 5′-TGCAGCTATACCCAGGCTGG-3′ |
| SEQ ID NO: 8 | TCF1 R | 5′-CCTCGACCGCCTCTTCTTC-3′ |
| SEQ ID NO: 9 | MYCF | 5′-TACCCTCTCAACGACAGCAG-3′ |
| SEQ ID NO: 10 | MYCR | 5′-TCTTGACATTCTCCTCGGTG-3′ |
| SEQ ID NO: 11 | WNT1 F | 5′-CGGCGTTTATCTTCGCTATC-3′ |
| SEQ ID NO: 12 | WNT1 R | 5′-TTCGATGGAACCTTCTGAGC-3′ |
| SEQ ID NO: 13 | WNT2 F | 5′-AAAGAAGATGGGAAGCGCCA-3′ |
| SEQ ID NO: 14 | WNT2 R | 5′-TTCATCAGGGCTCTGGCATC-3′ |
| SEQ ID NO: 15 | WNT3A F | 5′-TGTTGGGCCACAGTATTCCT-3′ |
| SEQ ID NO: 16 | WNT3A R | 5′-GGGCATGATCTCCACGTAGT-3′ |
| SEQ ID NO: 17 | WNT6 F | 5′-CAGCCCCTTGGTTATGGAC-3′ |
| SEQ ID NO: 18 | WNT6 R | 5′-AACTGGAACTGGCACTCTCG-3′ |
| SEQ ID NO: 19 | WNT8A F | 5′-AGGCTGAGAAGTGCTACCAGA-3′ |
| SEQ ID NO: 20 | WNT8A R | 5′-CCATTGTTTGACCCATCACA-3′ |
| SEQ ID NO: 21 | FZD1 F | 5′-CATCGTCATCGCCTGCTACT-3′ |
| SEQ ID NO: 22 | FZD1 R | 5′-TAGCGTAGCTCTTGCAGCTC-3′ |
| SEQ ID NO: 23 | FZD2 F | 5′-CTTCTCACAGGAGGAGACGC-3′ |
| SEQ ID NO: 24 | FZD2 R | 5′-AAATGATAGGCCGCTCTGGG-3′ |
| SEQ ID NO: 25 | FZD3 F | 5′-ACAGCAAAGTGAGCAGCTACC-3′ |
| SEQ ID NO: 26 | FZD3 R | 5′-CTGTAACTGCAGGGCGTGTA-3′ |
| SEQ ID NO: 27 | FZD4 F | 5′-TCAAGAGACGCTGTGAACCC-3′ |
| SEQ ID NO: 28 | FZD4 R | 5′-GGTCGTTCTGTGGTGGGAAT-3′ |
| SEQ ID NO: 29 | FZD5 F | 5′-GCACAACCACATCCACTACG-3′ |
| SEQ ID NO: 30 | FZD5 R | 5′-GCACAACCACATCCACTACG-3′ |
| SEQ ID NO: 31 | FZD6 F | 5′-AGGCTTGCACCGTTTTGTTC-3′ |
| SEQ ID NO: 32 | FZD6 R | 5′-TGCTCGATGGCTTCACAACT-3′ |
| SEQ ID NO: 33 | FZD7 F | 5′-CGCCTCTGTTCGTCTACCTC-3′ |
| SEQ ID NO: 34 | FZD7 R | 5′-TCATGATGGTGCGGATACGG-3′ |
| SEQ ID NO: 35 | FZD8 F | 5′-CGCCTCTGTTCGTCTACCTC-3′ |
| SEQ ID NO: 36 | FZD8 R | 5′-TCATGATGGTGCGGATACGG-3′ |
| SEQ ID NO: 37 | FZD9 F | 5′-TGCTCACCTTCTTGCTGGAG-3′ |
| SEQ ID NO: 38 | FZD9 R | 5′-GCCAGCGAGTAGACGTTGTA-3′ |
| SEQ ID NO: 39 | FZD10 F | 5′-AAGAAGAGCCGGAGAAAACC-3′ |
| SEQ ID NO: 40 | FZD10 R | 5′-GACTGGGCAGGGATCTCATA-3′ |
| SEQ ID NO: 41 | LRP5 F | 5′-CACCACCTTCTTGCTGTTCA-3′ |
| SEQ ID NO: 42 | LRP5 R | 5′-GCTTTGACGTTCCTCAGTCC-3′ |
| SEQ ID NO: 43 | LRP6 F | 5′-GACTGGGTTGCACGAAATCT-3′ |
| SEQ ID NO: 44 | LRP6 R | 5′-CGGGGTTCCTCTAAGTCCTC-3′ |

### In Vivo Experiments

NSG mice are obtained from the Jackson Laboratory and maintained at the BCM animal care facility. Mice are injected intravenously (IV) with 2×10⁵ luciferase-transduced Daudi lymphoma cells to initiate tumor growth. On day 3, mice are injected IV with 4×10×10⁶ CD19-CAR-NKTs followed by intraperitoneal (IP) injection of IL-2 (1,000 U/mouse) only or a combination of IL-2 (1,000 U/mouse) and IL-21 (50 ng/mouse) every other day for two weeks. To test the anti-neuroblastoma effect of CAR.GD2-NKTs, mice are injected i.v. with 1×10⁶ luciferase-transduced CHLA-255 neuroblastoma cells to initiate tumor growth. On day 7, mice are injected i.v. with 4×10⁶ CAR.GD2-NKTs followed by i.p. injection of IL-2 (2000 U/mouse) every other day for 2 wk. Tumor growth is assessed once per week by bioluminescent imaging (Small Animal Imaging core facility, Texas Children's Hospital).

### Statistics

The Shapiro-Wilk test is used to assess normality of continuous variables. Normality is rejected when the P value is less than 0.05. For non-normally distributed data, the Mann-Whitney U test is used to evaluate differences in continuous variables between two groups. To evaluate differences in continuous variables, a two-sided paired Student's t-test is used to compare two groups, one-way ANOVA with post-test Bonferroni correction is used to compare more than two groups, and two-way ANOVA with Sidak's post-hoc test is used to compare in a two-by-two setting. Survival is analyzed using the Kaplan-Meier method with the log-rank (Mantel-Cox) test to compare two groups. Statistics are computed using GraphPad Prism 7 (GraphPad Software, San Diego, CA). Differences are considered significant when the P value was less than 0.05.

### Example 2: WNT Signaling is Elevated in CD62L+ Cells and Activation of the Wnt Signaling Pathway Prevents Exhaustion

Gene-set enrichment analysis (GSEA) plot shows enrichment for a central memory CD4+ T cell signature (Abbas et al., 2009) in CD62L+ NKTs. See FIG. 1.

CD62L+ NKTs express elevated levels of Wnt transcriptional activator LEF1 compared to CD62L− NKTs. LEF1 expression in ex vivo-expanded primary NKTs is analyzed by intracellular flow cytometry on day 12 after antigenic stimulation with uGalCer-pulsed autologous PBMCs. NKTs are gated into CD62L+ and CD62L− populations and LEF1 expression of each group is measured. A representative histogram from one of seven donors (left) and mean±SEM of LEF1+ percentage for all donors (n=7, right) are shown. ***P<0.001, paired Student's t test. See FIG. 2. As shown in FIG. 2b, LEF1 is expressed at elevated levels in CD62L+ cells compared to CD62L− cells.

LEF1, but not TCF1 expression distinguishes CD62L+ from CD62L− NKTs. Co-expression of LEF1 and TCF1 in ex vivo-expanded NKTs is analyzed by intracellular flow cytometry. NKTs are gated into CD62L− (left) and CD62L+ (right) populations. Plots are from a representative of three donors (FIG. 2).

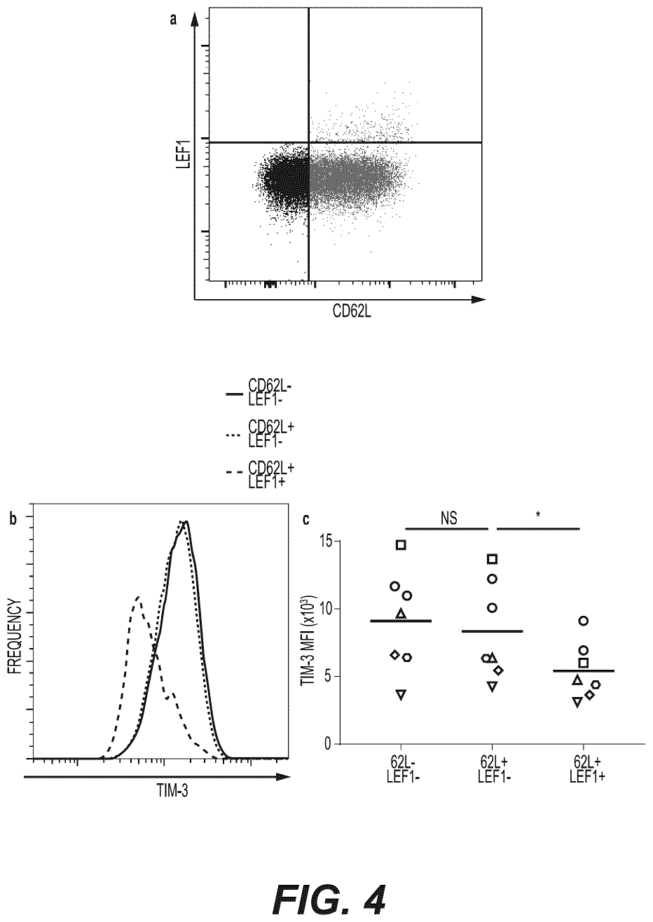

LEF1+ NKTs are phenotypically less exhausted than LEF1− NKTs. NKTs are gated into CD62L− LEF1−, CD62L+ LEF1− and CD62L+ LEF1+ populations (FIG. 4a), and TIM-3 expression is measured in each group. Representative histogram from one of seven donors (FIG. 4b) and mean±SEM of TIM-3 MFI for all donors (n=7, FIG. 4c) are shown. *P<0.05, ns: not significant, one-way ANOVA with Sidak's post-test. As shown, TIM-3 express, a marker for exhaustion is reduced in LEF1 expression cells.

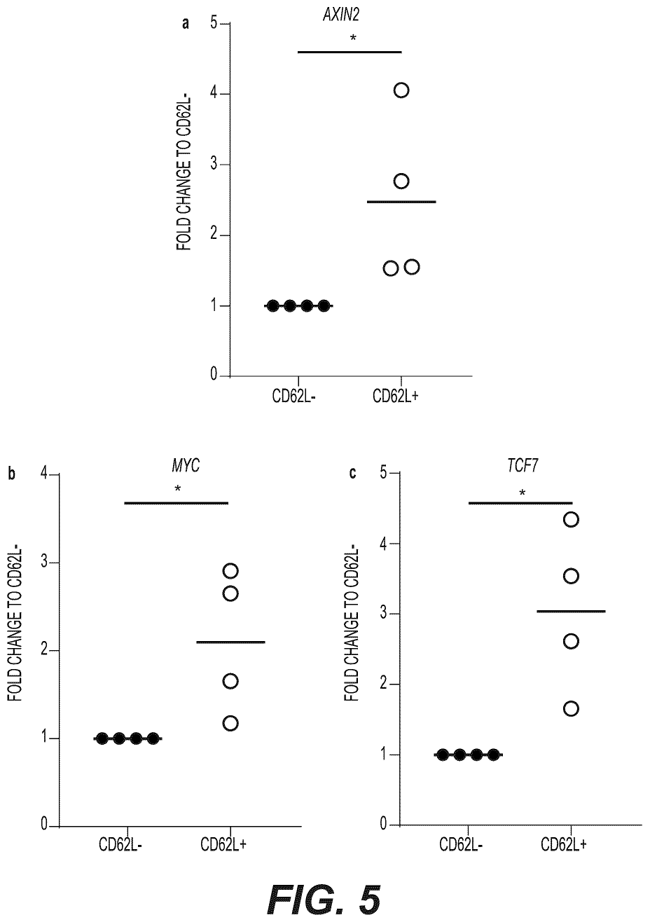

Basal Wnt/β-catenin signaling is elevated in CD62L+ compared to CD62L− NKTs. NKTs are magnetically sorted into CD62L+ and CD62L− subsets followed by RNA isolation. Expression of Wnt target genes is measured by quantitative PCR and calculated by the ddCt method. A summary of four donors is shown (FIG. 5). *P<0.05, Student's t test.

FIG. 5 is a summary of the results of four donors for the expression of Wnt target genes measured by quantitative PCR and calculated by the ddCt method of magnetically sorted CD62L+ and CD62L− subsets. Axin2 (a), Myc (b) and TCF7 (c).

Basal Wnt/β-catenin signaling is elevated in CD62L+ compared to CD62L− NKTs. NKTs are magnetically sorted into CD62L+ and CD62L− subsets followed by RNA isolation. Expression of Wnt target genes are measured by quantitative PCR and calculated by the ddCt method. A summary of four donors is shown (FIG. 5: Axin2 (a), Myc (b) and TCF7 (c)). *P<0.05, Student's t test.

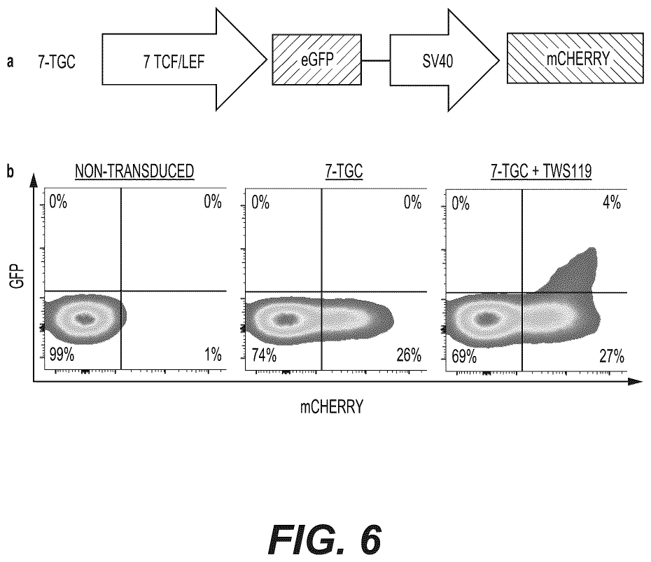

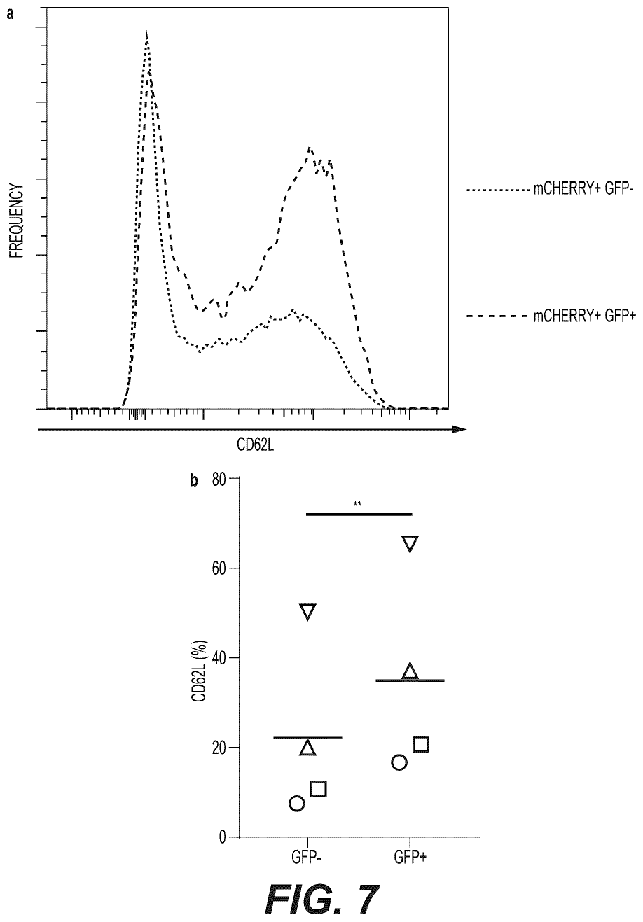

Wnt/β-catenin signaling activity can be detected in NKTs transduced with the 7-TGC reporter. NKTs are transduced with the 7-TGC reporter (FIG. 6) on day 12 after ex vivo stimulation to monitor Wnt signaling activity. Transduction and Wnt activity is measured by detecting mCherry and GFP, respectively. Dot-plots of non-transduced (FIG. 6a), 7-TGC-transduced (FIG. 6b) and 7-TGC-transduced NKTs treated with 10 μM Wnt activator TWS119 overnight (FIG. 6c) are shown.

CD62L+ NKTs are enriched in the Wnt-active subset. mCherry+ NKTs are gated into GFP− and GFP+ subsets, and CD62L expression is measured in each group (FIG. 7). A representative histogram from one of four donors (FIG. 7a) and mean CD62L+ percentage for all donors (n=4, FIG. 7b) are shown. Each symbol denotes an individual donor. **P<0.01, Student's t test.

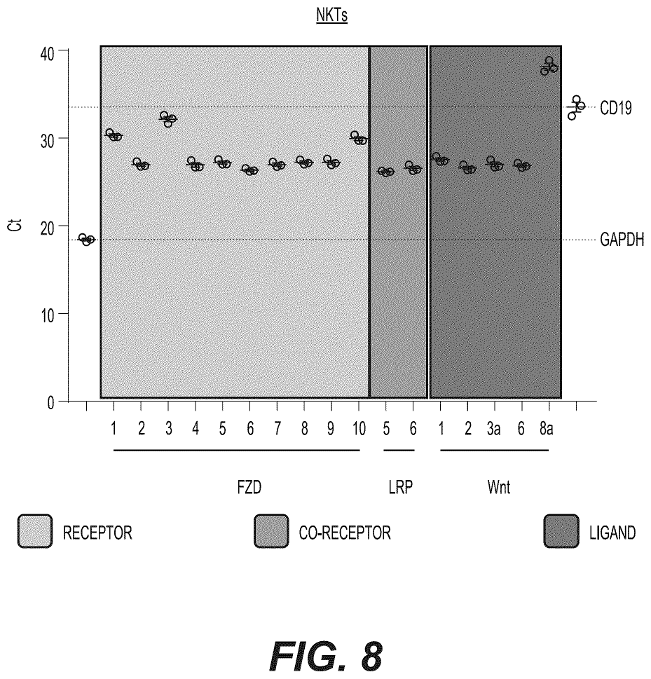

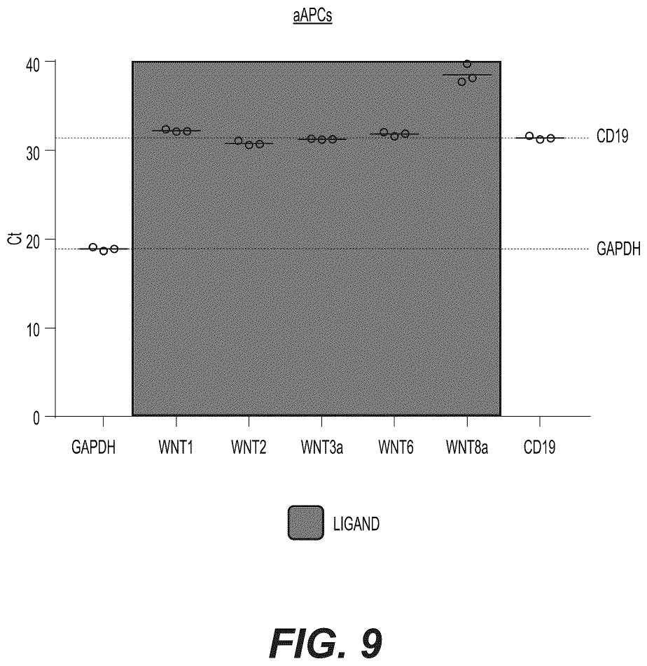

NKTs express components necessary to drive Wnt/β-catenin signaling. NKTs are expanded ex vivo for 10 days followed by RNA isolation. Wnt receptor, co-receptor, and ligand mRNA expression are measured by quantitative PCR. GAPDH and CD19 are used to detect positive and negative expression, respectively. Genes with Ct values that fall within the range of GAPDH and CD19 are considered significantly expressed. A summary of three independent experiments, each with an individual donor, is shown in FIG. 8.

K562-based artificial antigen presenting cells (aAPCs) do not express Wnt ligands to drive Wnt/β-catenin signaling. RNA is isolated from K562-based aAPCs followed by quantitative PCR to detect Wnt ligand expression as described in above and shown in FIG. 8. A summary of three independent experiments is shown.

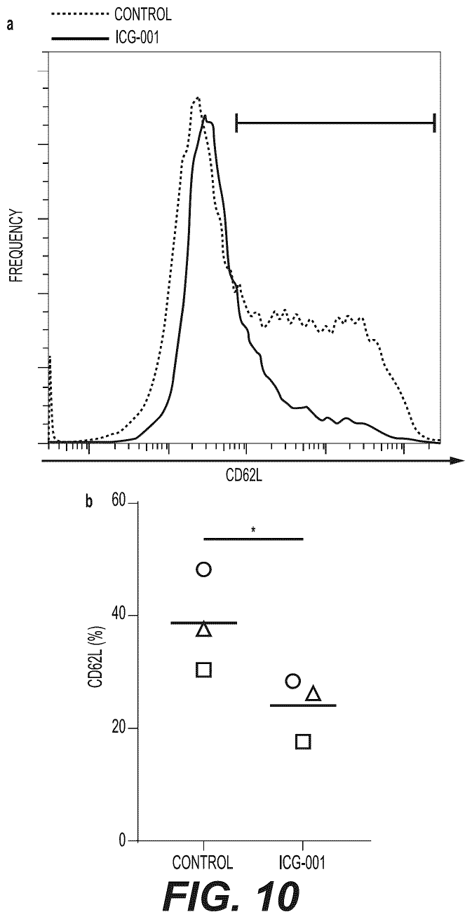

Basal Wnt signaling is important for development of CD62L+ NKTs ex vivo. NKTs are treated with 3 μM of Wnt inhibitor ICG-001 after stimulation with uGalCer-pulsed autologous PBMCs starting at day 0 for three days. CD62L expression is examined on day 12 after antigenic stimulation. A representative histogram from one of three donors (left) and mean of CD62L+ percentage for all donors (n=3, right) is shown in FIG. 10. Each symbol denotes an individual donor. *P<0.05, Student's t test.

### Example 3: Activation of WNT Signaling Promotes Development of CD62L+ Nkt Cells

Activation of Wnt signaling with recombinant Wnt3a promotes development of CD62L+ NKTs. NKTs are stimulated as described in Example 2 but treated with three separate doses of PBS or 500 ng/mL Wnt3a given on day 0, 3 and 7 after antigenic stimulation. CD62L expression is examined by flow cytometry on day 12 after antigenic stimulation. A representative histogram from one of three donors (left) and mean of CD62L+ percentage for all donors (n=3, right) are shown. Each symbol denotes an individual donor. *P<0.05, Student's t test. FIG. 11 shows that activation of Wnt signaling via Wnt ligand increases the frequency of cells that are CD62L+.

Activation of Wnt signaling via GSK30 inhibition promotes development of CD62L+ NKTs. NKTs are stimulated as described in Example 2 but treated with one dose of DMSO or 5 μM of the GSK30 inhibitor TWS119 on day 7 after antigenic stimulation. CD62L expression is examined by flow cytometry on day 12 after antigenic stimulation. A representative histogram from one of six donors (left) and mean of CD62L+ percentage for all donors (n=6, right) are shown in FIG. 12. Each symbol denotes an individual donor. **P<0.01, Student's t test. As shown in FIG. 12, inhibition of the Wnt signaling pathway inhibitor GSK3b leads to an increase in CD62L+ NKT cells. See Wu and Pan (2010). That is, inhibition of inhibition of the Wnt pathway increases the frequency of CD62L+ NKT cells.

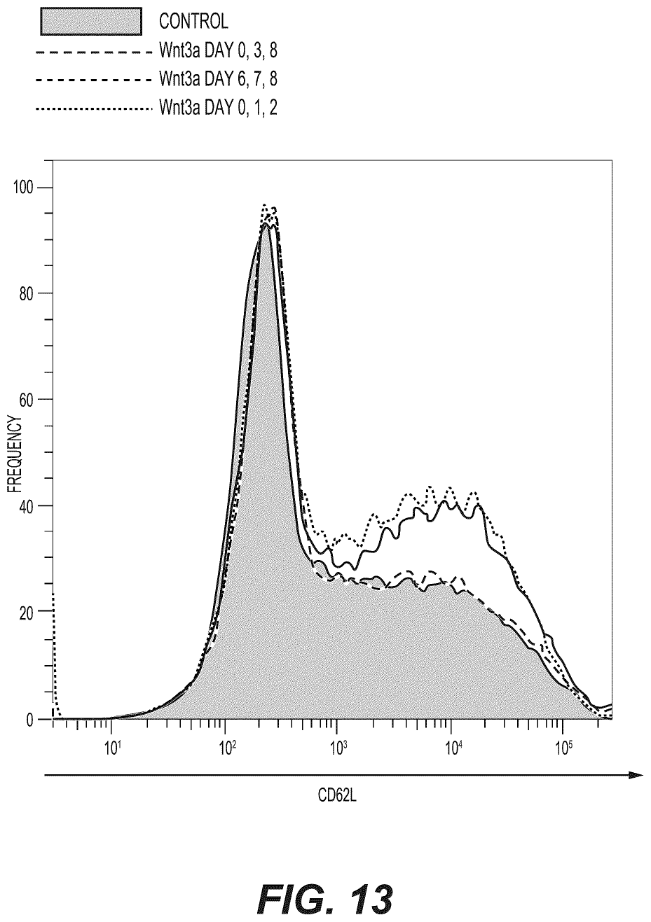

Wnt signaling acts during early stages of TCR stimulation to promote development of CD62L+ NKTs. NKTs are stimulated with uGalCer-pulsed autologous PBMCs and treated with three doses of PBS or 500 ng/ml Wnt3a in early (days 0,1,2), late (days 6,7,8) or throughout (days 0,3,8) expansion. CD62L expression is examined by flow cytometry on day 12 after antigenic stimulation and the results shown in FIG. 13.

### Example 4: Wnt Signaling and Growth Factor Signaling Act Additively to Promote Development of CD62L+ NKT Cells

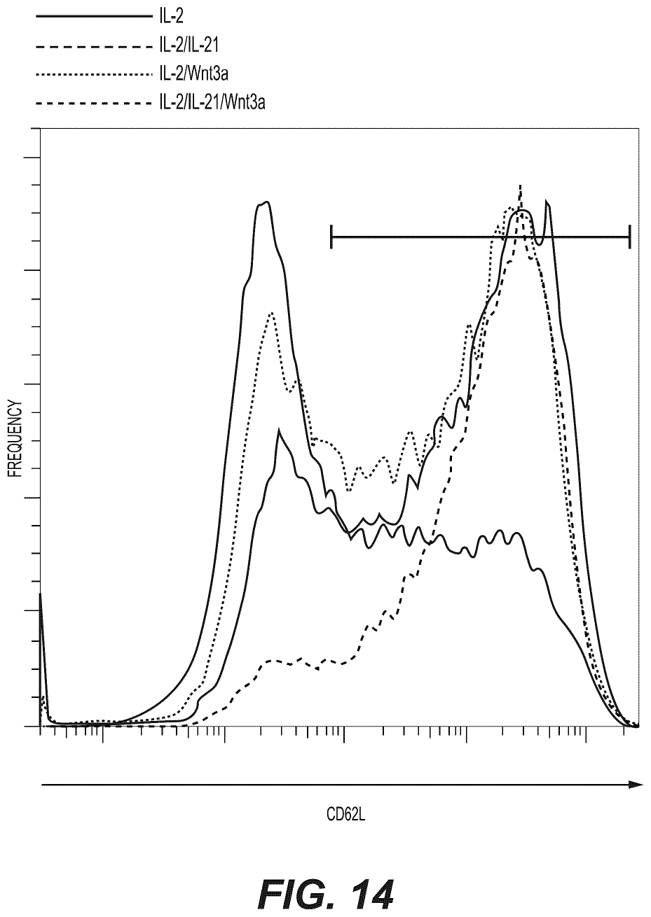

Wnt3a works additively with IL-21 to increase CD62L frequency. Following primary stimulation, NKTs are cultured with IL-2 or IL-2/IL-21 for 12 days. In each group, PBS or 500 ng/ml Wnt3a treatment is administered on days 0, 1, and 2 after antigenic stimulation. CD62L expression is analyzed by flow cytometry at the end of expansion. A representative histogram from one of three donors is shown in FIG. 14.

### Example 5: Loss of Wnt Signaling Decreases the Frequency of CD62L+ NKT Cells

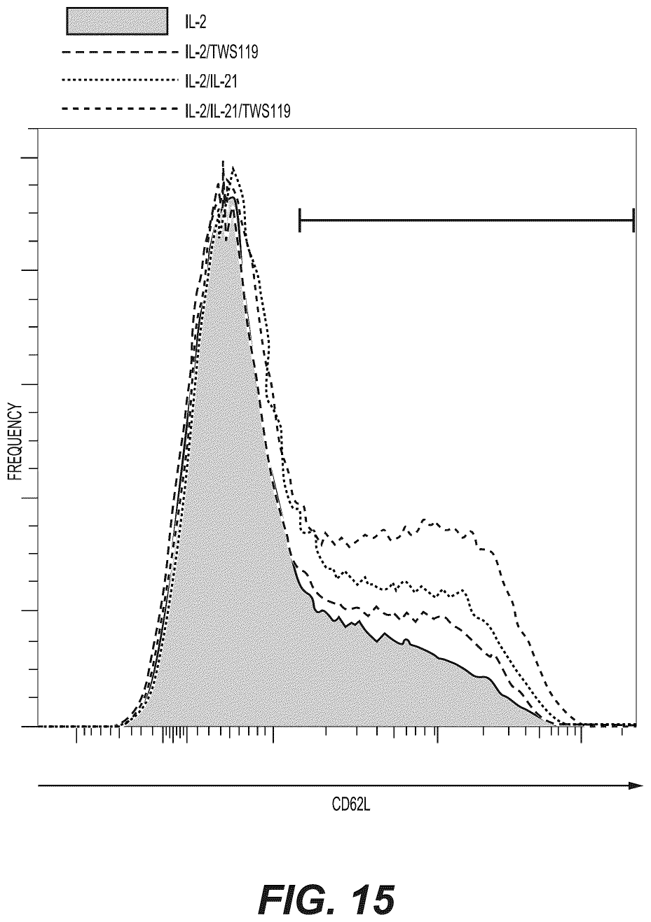

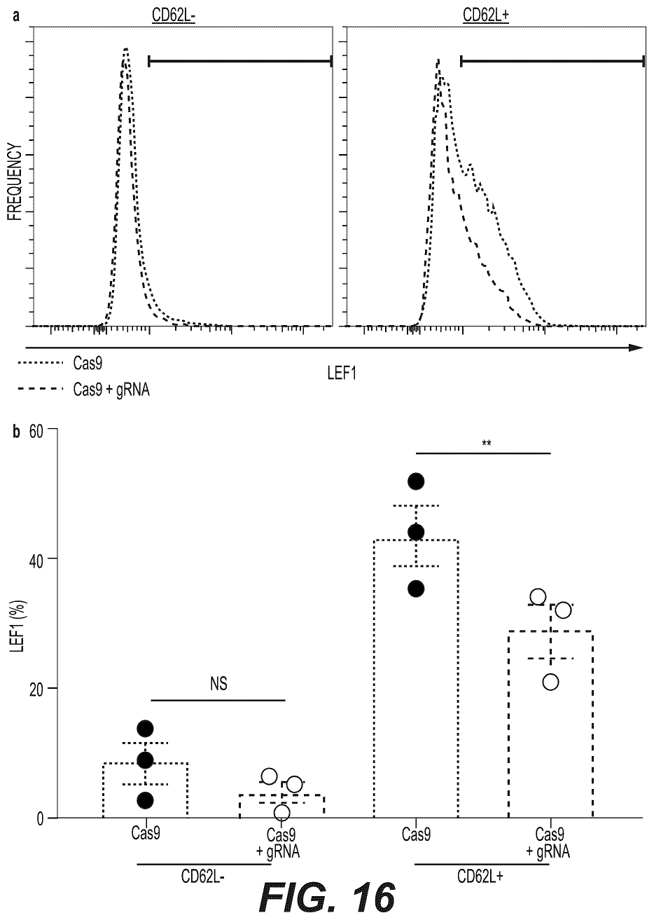

CRISPR/Cas9-mediated LEF1 knockout (KO) decreases the frequency of CD62L+ NKTs. NKTs are electroporated with Cas9 with or without guide RNA targeting LEF1 on day of ex vivo expansion. CRISPR/Cas9-mediated LEF1 KO is evaluated by intracellular flow cytometry on day 3 after electroporation. NKTs are gated into CD62L− and CD62L+ populations and LEF1 expression is analyzed. A representative histogram from one of three donors (FIG. 15a) and mean±SEM of CD62L+ percentage for all donors (n=3, FIG. 15b) are shown. **P<0.01, ns: not significant, Student's t test. The decrease in frequency of CD62L+ cells by knockdown of LEF1 indicates that Wnt signaling is required for production of CD62L+ NKT cells.

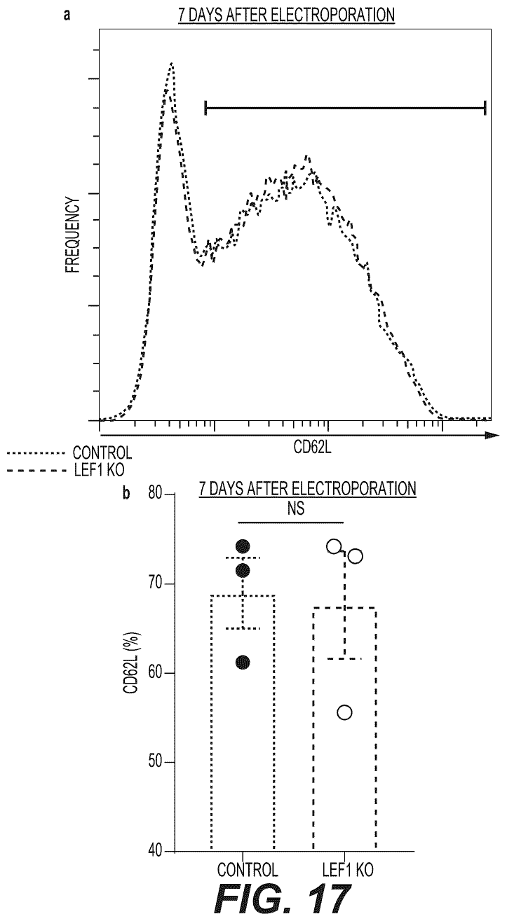

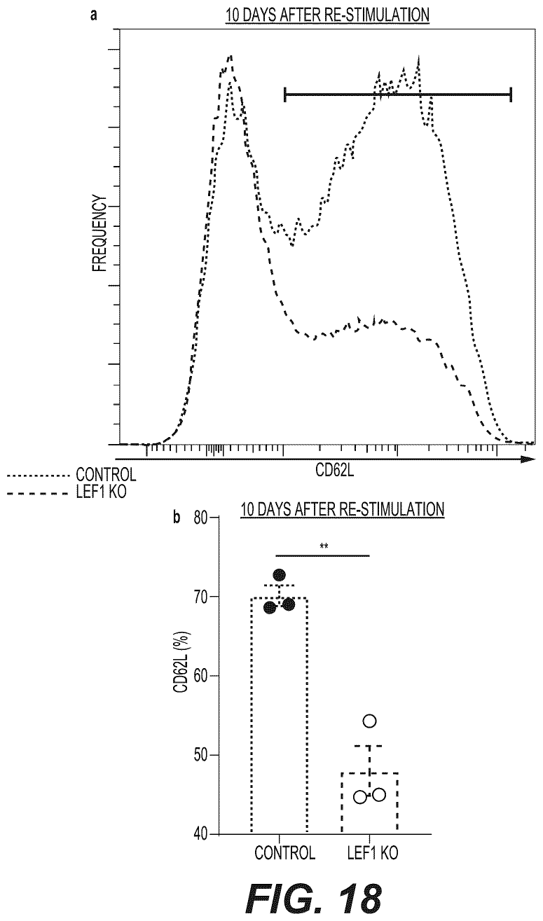

LEF1 KO only impacts CD62L+ subset development after antigenic stimulation. CD62L expression is analyzed by flow cytometry in electroporated NKTs before (Day 7 after electroporation, FIG. 17a). As shown, reduction of LEF1 expression does not have a significant effect on CD62L+ NKT cell frequency prior to stimulation. In contrast, 10 days after antigenic re-stimulation (Day 10 after secondary stimulation, FIG. 18), loss of LEF1 results in significant decreases in CD62L+ NKT cells. Representative histograms from one of three donors (FIG. 17a, FIG. 18a) and mean±SEM of CD62L+ percentage for all donors (n=3, FIG. 17b and FIG. 18b) are shown. **P<0.01; ns: not significant, paired Student's t test. These results show that activation of Wnt signaling is required for the CD62L+ subset after antigenic stimulation.

### Example 6: Activation of WNT Signaling Maintains NKT Expansion Capacity and Alleviates NKT Cell Exhaustion

LEF1 overexpression in NKTs can be achieved using a gammaretroviral approach. A gammaretroviral construct that overexpresses the long isoform of LEF1 is prepared for expression in NKTs (FIG. 19, top, GFP.LEF1). NKTs are transduced with GFP.FFLuc or the GFP.LEF1 construct two days after secondary stimulation using uGalCer-pulsed aAPCs. On day 12 of expansion, GFP expression is evaluated by flow cytometry (FIG. 19a) and LEF1 expression is analyzed separately by intracellular flow cytometry (FIG. 19b). Representative dot-plots from one of three donors are shown.

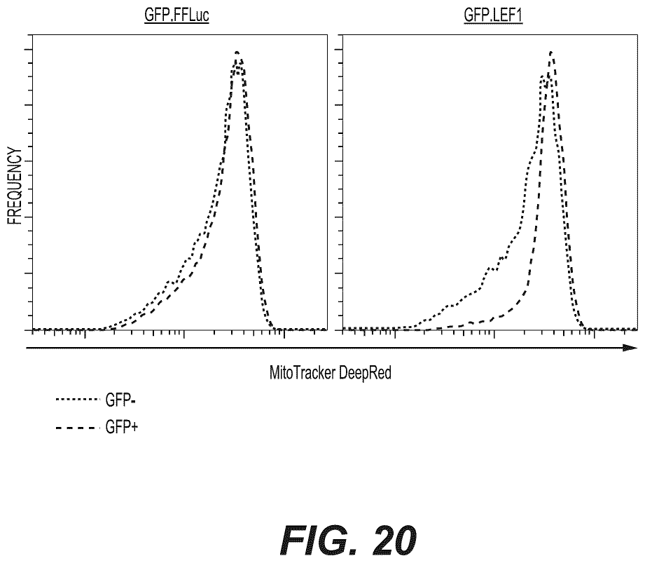

LEF1 overexpression increases mitochondrial mass in NKTs. Mitochondrial mass of GFP.FFLuc- and GFP.LEF1-transduced NKTs as prepared above is analyzed by MitoTracker staining and flow cytometry. NKTs are gated into non-transduced (GFP−) and transduced (GFP+) populations and MitoTracker DeepRed staining is compared. Representative histograms from one of two donors are shown in FIG. 20.

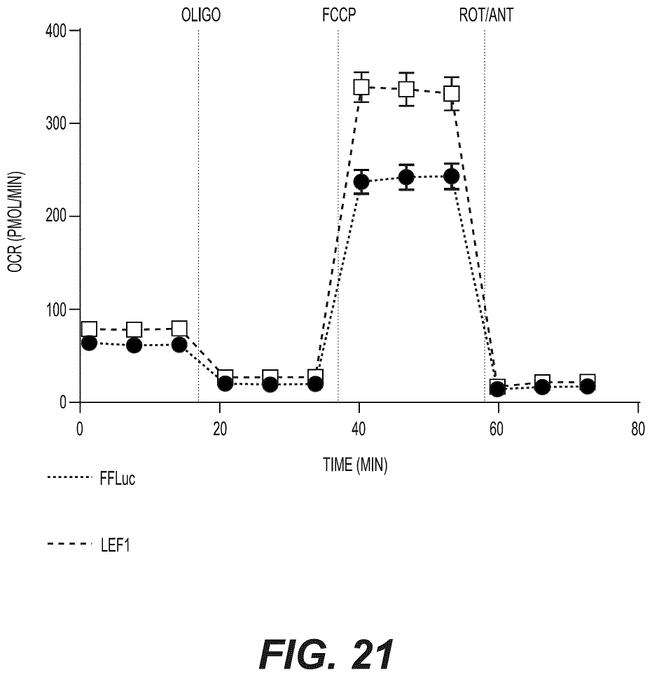

LEF1 overexpression increases mitochondrial capacity in NKTs. Oxygen consumption rate (OCR) of GFP.FFLuc- and GFP.LEF1-transduced NKTs as prepared above is measured by Seahorse assay under basal conditions and in response to oligomycin, FCCP, and rotenone and antimycin A (Rot/Ant). See FIG. 21. Results are representative from one of two donors tested.

LEF1 overexpression maintains NKT cell expansion capacity after repeated antigen challenge. NKTs transduced with GFP.FFLuc and GFP.LEF1 are repeatedly challenged with CDld+ J32 leukemia cells at a 1:1 ratio every three days, shown diagrammatically in FIG. 22, top. NKTs are enumerated at each cycle using counting beads and flow cytometry. Fold change in expansion for each cycle is shown in FIG. 22, bottom.

LEF1 overexpression promotes effective control of tumor burden by NKTs after repeated antigen challenge. After the seventh cycle of repeated killing assay as shown in above and in FIG. 22, NKTs are challenged with J32 cells at a 1:50 ratio for three days. Tumor cell number is determined using counting beads and flow cytometry. Tumor reduction percentage is shown in FIG. 23.

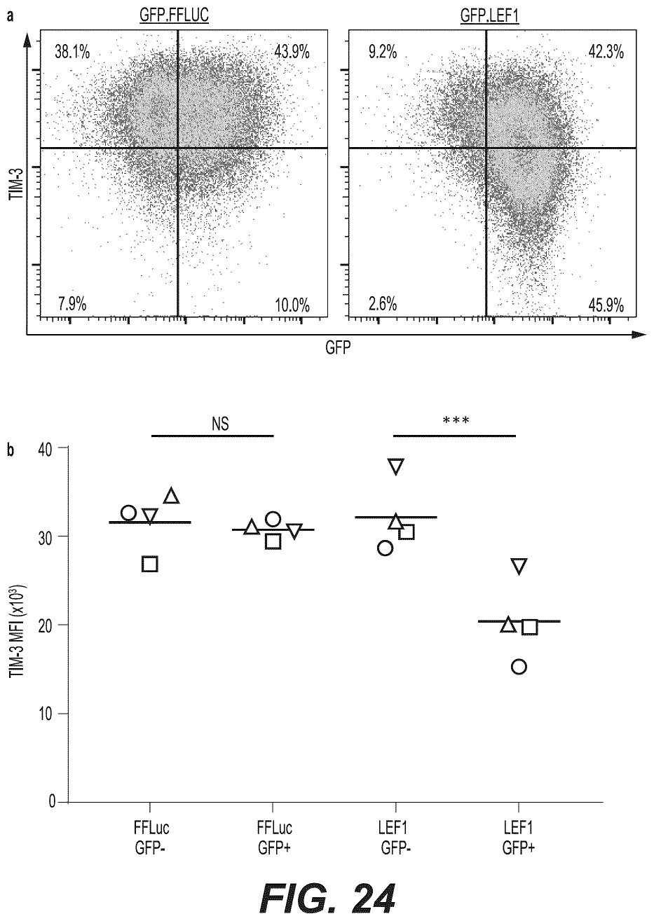

LEF1 overexpression alleviates NKT cell exhaustion after repeated antigen challenge. After the fifth cycle of repeated killing assay as in FIG. 22, NKTs are isolated from antigen stimulation and rested for a total of six days. TIM-3 expression in relation to GFP is then examined by flow cytometry. Representative dot plots from one of four donors (FIG. 24a) and mean TIM-3 MFI for all donors (n=4, FIG. 24b) are shown. Each symbol denotes an individual donor. ***P<0.001, ns: not significant, one-way ANOVA and Sidak's post-test. The results are presented in Table 3.

TABLE 3
TIM-3 MFI

| | BN32 | BN97 | BN125 | BN134 |
|---|---|---|---|---|
| FFLuc GFP− | 32637 | 26852 | 34583 | 32256 |
| FFLuc GFP+ | 31943 | 29208 | 31902 | 30555 |
| LEF1 GFP− | 28640 | 30498 | 31699 | 37802 |
| LEF1 GFP+ | 15279 | 19742 | 20041 | 26582 |

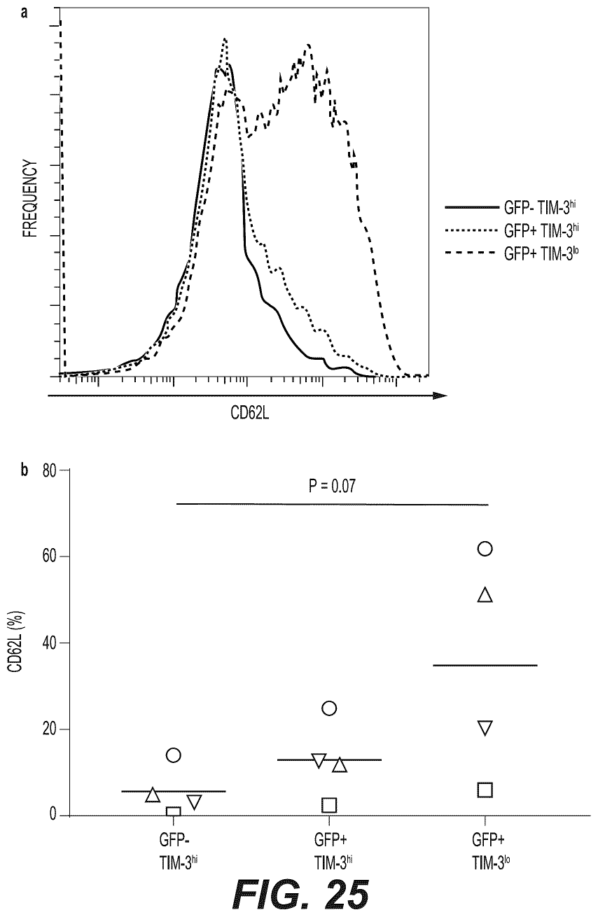

LEF1 overexpression maintains CD62L+ central memory NKTs after repeated antigen challenge. NKTs are rested as described in above and shown in FIG. 24 and then gated into GFP-TIM-3^{hi}, GFP+ TIM-3^{hi}, and GFP+ TIM-3^{lo} populations. CD62L expression is evaluated in each group by flow cytometry. A representative histogram from one of four donors (FIG. 25a) and mean CD62L percentage for all donors (n=4, FIG. 25b) are shown. Each symbol denotes an individual donor. One-way ANOVA and Sidak's post-test.

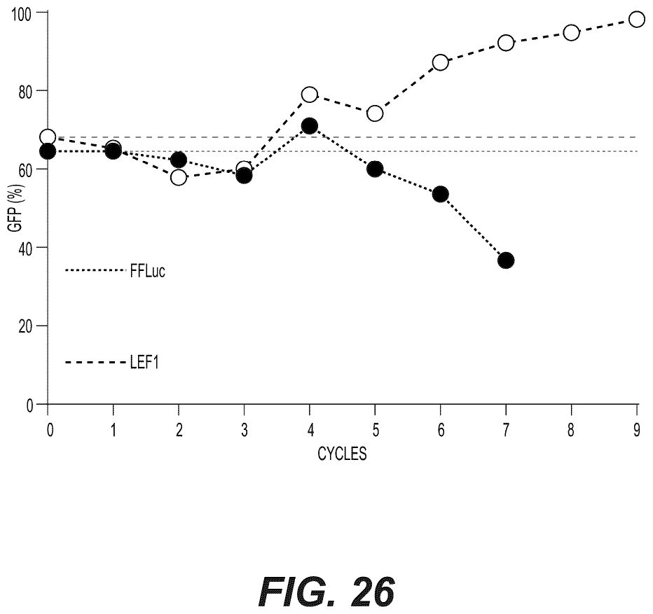

(y) NKTs overexpressing LEF1 show selective advantage over GFP.FFluc control cells over the course of repeated antigen challenge. NKTs are repeatedly challenged as described above and shown in FIG. 22. Enrichment of transduced cells is monitored by GFP expression using flow cytometry, FIG. 26. Shown is representative result from one of four donors.

### Example 7: CAR Constructs Encoding for Co-Expression of LEF1

CAR.GD2 constructs encoding co-expression of LEF1 are shown diagrammatically in FIG. 27. The LEF1 long isoform (NM_016269.5) was inserted after a 2A sequence downstream of the CD28 or 4-1BB endodomain, or IL-15 if present.

NKTs transduced with various LEF1-containing constructs express both LEF1 and CAR.GD2. NKTs are transduced with parental or LEF1-containing CAR.GD2 constructs two days after secondary stimulation with uGalCer-pulsed aAPCs. On day 12 of expansion, surface expression of CAR.GD2 and intracellular expression of LEF1 are determined by flow cytometry. Representative dot plots show LEF1 expression in relation to CAR expression from one of two donors.

### Example 8: LEF1 Overexpression Promotes Late-Stage NKT Expansion after Tumor Cell Killing

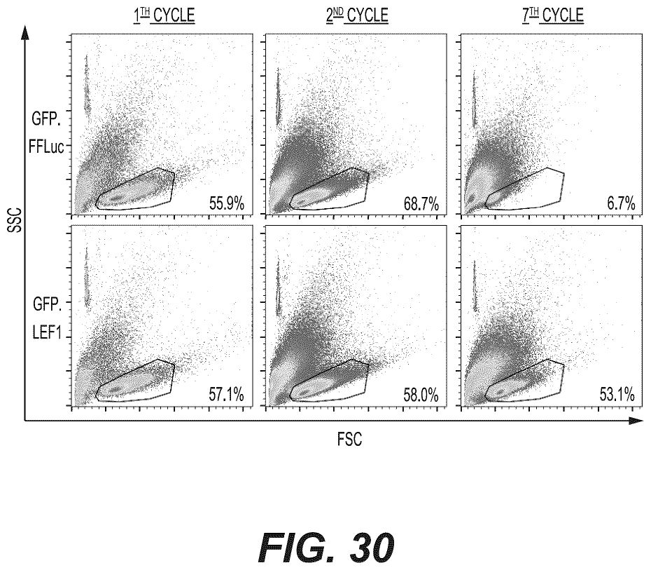

CD1D NKT cells are isolated as described above and 1×10⁶ cells are challenged with J32 tumor cells at a 1:1 ratio every three days (e.g., cycle). After three days, the expanded NKT cells are isolated, and a challenge with J32 tumor cells re-established. The results are FACS sorted and the plots presented. As shown, at the seventh cycle the LEF1 expressing NKT cells retain the ability to expand. See FIG. 30.

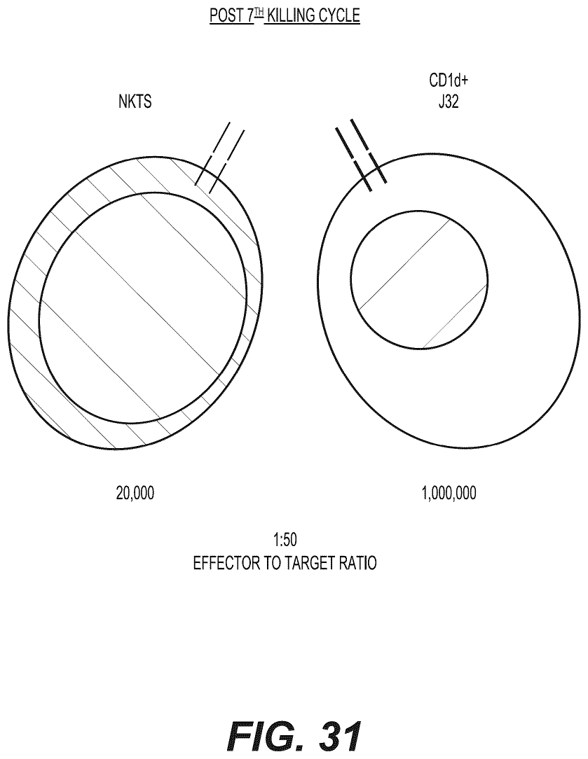

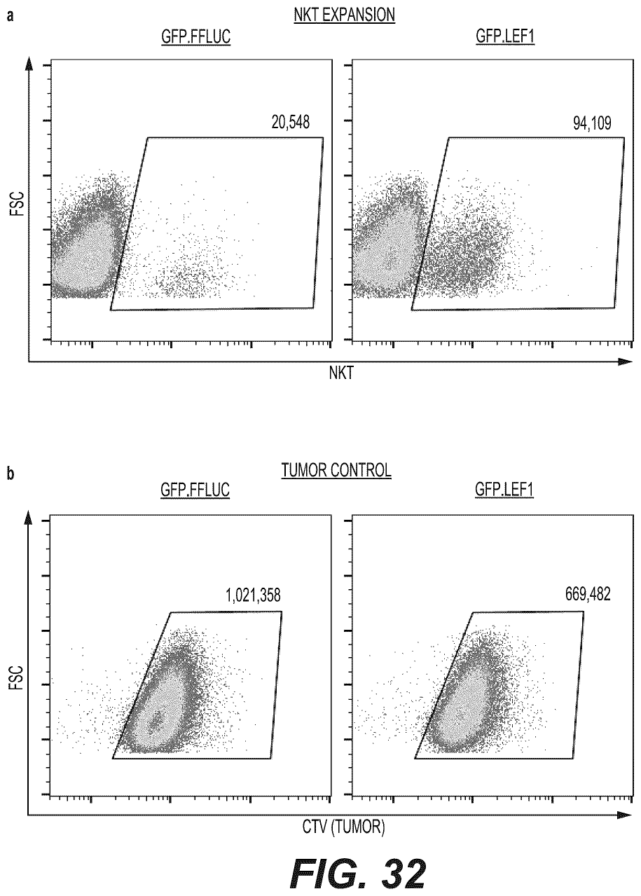

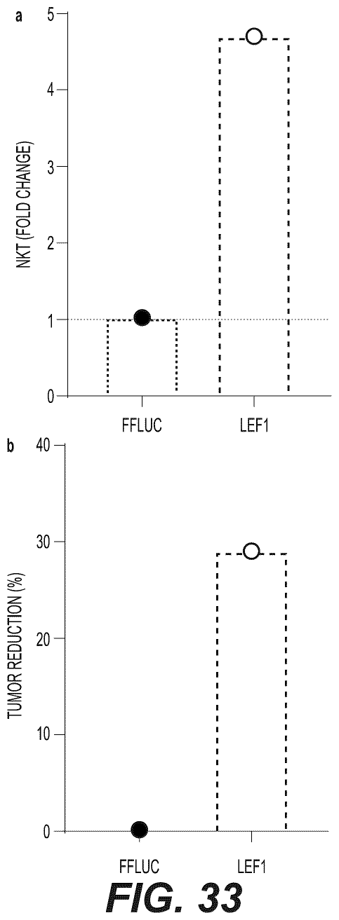

The expanded NKT cells retain effective tumor cell killing after seven cycles. After seven cycles, the expanded NKT cells are isolated and challenged with tumor cells at a 1:50 ratio as illustrated in FIG. 31. FIG. 32 presents dot plots of NKTs transduced with the GFP.FFLUC or GFP.LEF1. As shown in FIG. 32, panel a, LEF1 expressing cells retain the ability to expand. As shown in FIG. 32, panel b, the LEF1 expressing cells retain tumor killing ability while control NKT cells do not. FIG. 33 is a graphical representation of the results of FIG. 32.

### Example 9: LEF1 Crispr-CAS9 Gene Editing

Two guide RNAs (gRNAs) for the LEF1 gene (SEQ ID NO: 45, CCCGGAATAACTCGAGTAGG and SEQ ID NO:46 GTCACTGTAAGTGATGAGGG) are designed using CRISPRscan and COSMID algorithms (Moreno-Mateos et al., “CRISPRscan: designing highly efficient sgRNAs for CRISPR-Cas9 targeting in vivo,” Nat Methods 12, 982-988 (2015) and Cradick et al., “COSMID: A Web-based Tool for Identifying and Validating CRISPR/Cas Off-target Sites, “Mol Ther Nucleic Acids 3, e214 (2014). The LEF1 gene is genomically disrupted in NKTs according to the published protocol (Gundry et al., “Highly Efficient Genome Editing of Murine and Human Hematopoietic Progenitor Cells by CRISPR/Cas9,” Cell Rep 17, 1453-1461 (2016). Briefly, the 20-nt sequence complementary to the specific gene locus is incorporated into an oligonucleotide primer and used to amplify the gRNA scaffold from the PX458 plasmid (a gift from Feng Zhang; Addgene 48138). gRNAs are generated through in vitro transcription using the High-Yield RNA Synthesis Kit (NEB Bio Labs) from the DNA template following the manufacturer's instructions, and purified using the RNA Clean & Concentrator-25 kit (Zymo Research). The Neon Transfection System (ThermoFisher Scientific) is used to electroporate 5 μg of gRNA and 10 μg of Cas9 protein (PNA Bio) into 2×10⁶ of activated NKTs in 100 μL of buffer R using 3 1600-V 10-ms pulses. Following electroporation, NKTs are incubated in complete RPMI supplemented with 10% fetal bovine serum overnight. NKTs are then expanded in media supplemented with 200 U/mL IL-2.

### Example 10: LEF1 Overexpression Induces Central Memory Genes and Represses Genes Related to Exhaustion and Effector Phenotype

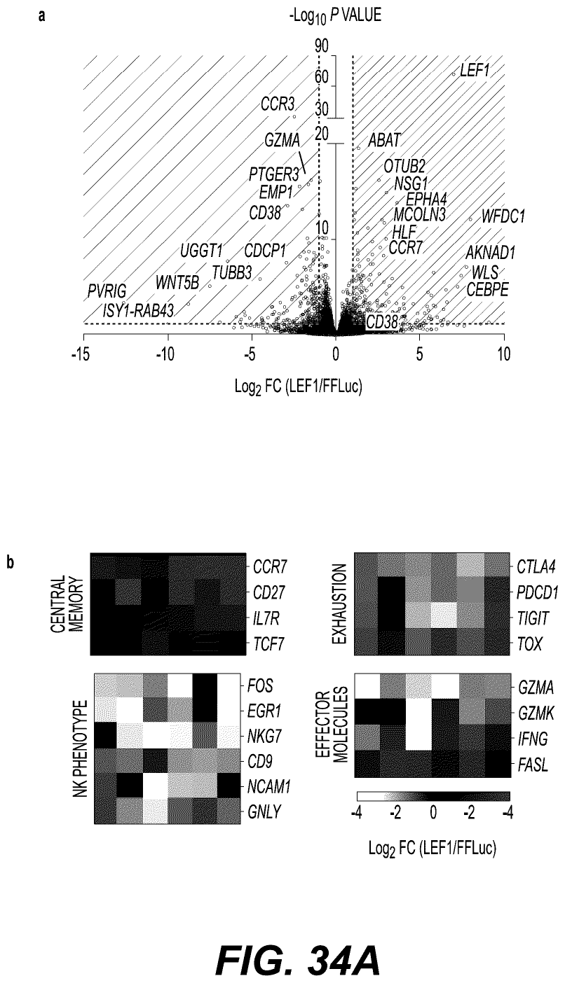

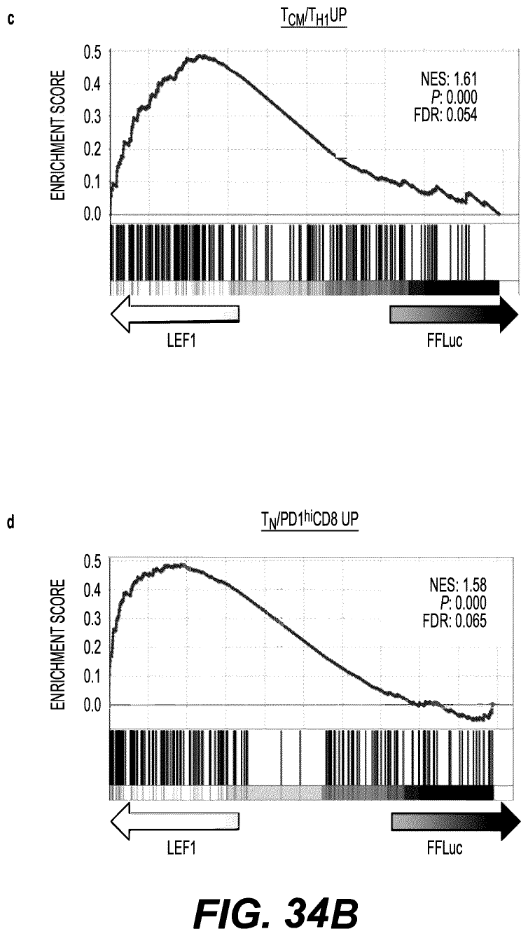

GFP.FFluc and GFP.LEF1 NKTs cells are collected 10 days after secondary stimulation and GFP positive cells are FACS sorted. RNA isolated from the sorted cells is processed for bulk RNA sequencing analysis. Differentially expressed genes (DEGs) of interest are grouped by shared phenotype/function. The results are plotted in FIG. 34A, panel a and heat maps show fold change in expression (LEF1/FFLuc) having a false discovery rate (FDR)<0.05 (FDR<0.20 for PDCD1) in LEF1-GFP+ and FFLuc-GFP+ cells (FIG. 34A, panel b). The results grouped by shared phenotype and function are presented for six donor are presented in Table 3.

As shown in FIG. 34B, panel c, LEF1 overexpression induces a central memory-like transcriptome signature and a less exhausted transcriptome signature as shown in FIG. 34B, panel d. FIG. 34B, panel c, presents a GSEA plot showing enrichment for a central memory T cell signature (Jeffrey et al., 2006) in LEF1-overexpressing NKTs. GSEA plots showing enrichment for a CD8 T cell signature with lower exhaustion levels (Duraiswamy et al., 2011) in LEF1-overexpressing NKTs is shown in FIG. 34B, panel d.

TABLE 4
Relative gene expression changes in LEF/FFLuc NKT cells

| | Donor 1 | Donor 2 | Donor 3 | Donor 4 | Donor 5 | Donor 6 |
|---|---|---|---|---|---|---|
| Central memory | | | | | | |
| CCR7 | 2.315614 | 1.843841 | 1.396828 | 3.232262 | 3.411612 | 3.38257 |
| CD27 | −0.173424 | 3.639276 | 0.60115 | 3.161936 | 1.978275 | 3.322008 |
| IL7R | 1.085254 | 0.547104 | 1.328313 | 1.470893 | 2.090878 | 2.185651 |
| TCF7 | 1.188626 | 0.482694 | 1.851472 | 1.197209 | 1.277515 | 0.340437 |
| NK-like | | | | | | |
| FOS | −2.227943 | −2.130403 | −1.538118 | −3.334606 | 0.39927 | −3.949277 |
| EGR1 | −2.43222 | −3.048757 | −1.062261 | −1.85922 | −0.232733 | −3.992186 |
| NKG7 | −0.184992 | −2.459956 | −3.359662 | −2.60333 | −1.167963 | −2.583877 |
| CD9 | −1.137423 | −1.369996 | −0.48822 | −1.714581 | −1.82673 | −1.657087 |
| NCAM1 | −0.869682 | −0.190734 | −2.782898 | −2.171811 | −2.10295 | 0.286216 |
| GNLY | −0.840321 | −1.631419 | −2.485367 | −1.16301 | −0.770509 | −1.284853 |
| Effector molecules | | | | | | |
| GZMA | −1.143286 | −1.445065 | −1.633473 | −1.338396 | −2.048814 | −1.434911 |
| GZMK | −1.138193 | 0.386415 | −1.727912 | −1.32027 | −1.568738 | −0.645463 |
| IFNG | −1.087194 | 0.111923 | −2.023556 | −2.470298 | −1.648857 | −0.736864 |
| FASL | −0.934671 | −0.351611 | −1.169181 | −0.703096 | −1.079037 | −0.664849 |
| Exhaustion | | | | | | |
| CTLA4 | −3.014733 | −1.519203 | −2.27583 | −3.095671 | −1.504323 | −1.599353 |
| PDCD1 | 0.237912 | 0.019296 | −3.082796 | −0.487679 | −1.597223 | −1.020446 |
| TIGIT | −1.442005 | −0.584228 | −3.382726 | −0.520653 | −0.83664 | −0.360597 |
| TOX | −0.375119 | −0.679339 | −0.654632 | −0.298288 | −0.651321 | −0.165938 |

### Example 11: Design and Expression of CAR-LEF1 Constructs

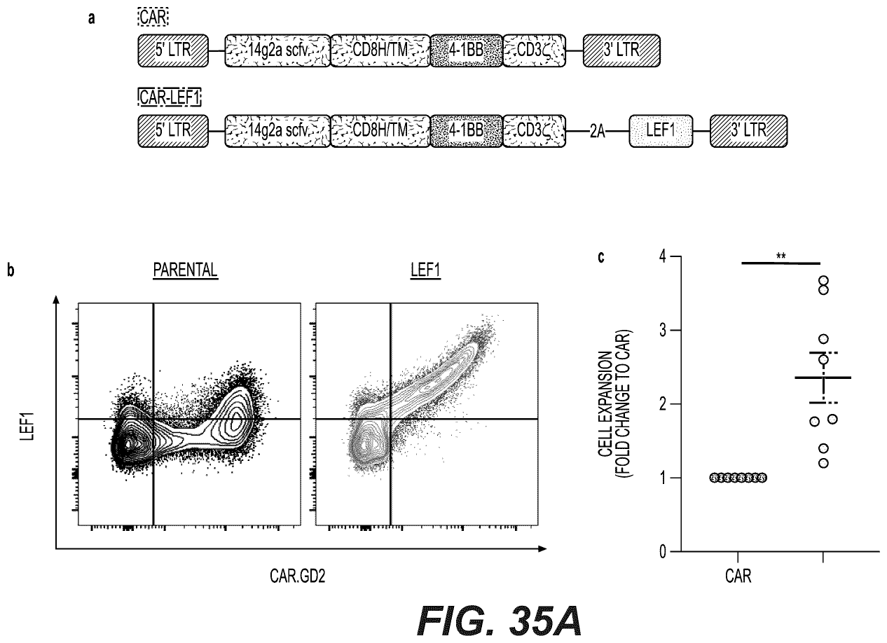

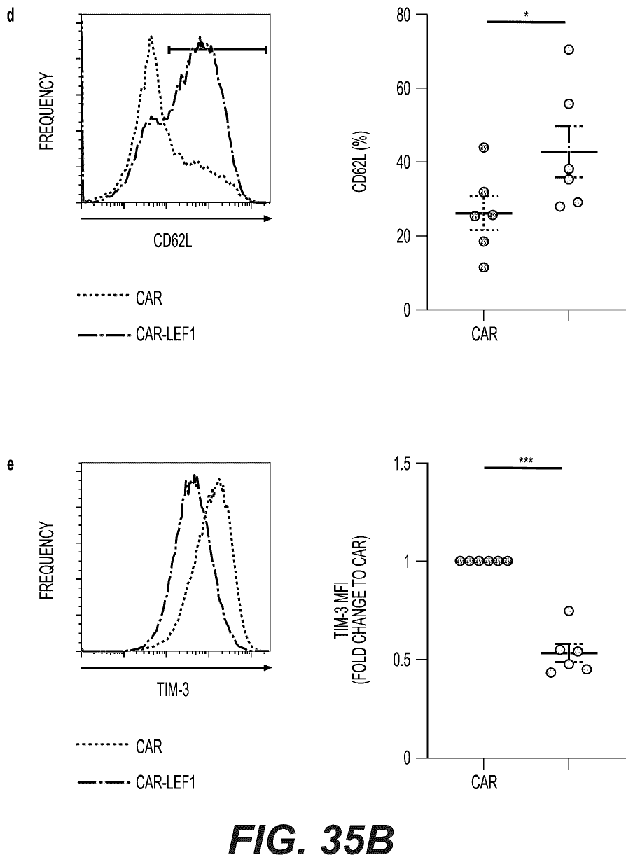

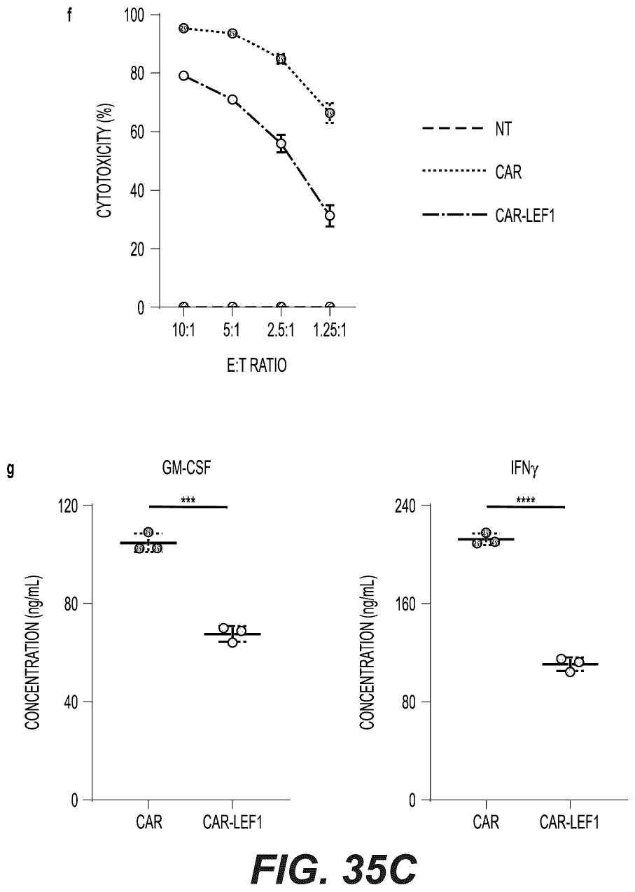

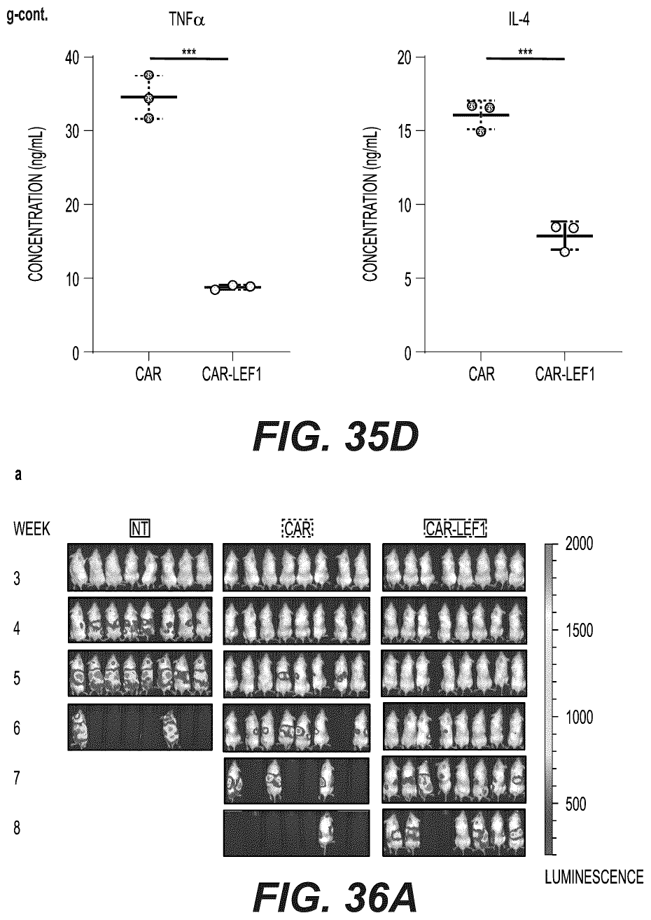

A schematic of an exemplary design of a CAR LEF1 construct is presented in FIG. 35A, panel a. The CAR constructs contain the anti-GD2 14g2a scFv, CD8 hinge and transmembrane domains, 4-1BB co-stimulatory domain, and CD3 zeta domain, with LEF1 following a 2A sequence (CAR-LEF1) or without (CAR).

The co-expression of CAR and LEF1 is determined. Two days after secondary stimulation with uGalCer-pulsed aAPCs, NKTs are transduced with parental or LEF1− containing CAR.GD2 constructs. After 10 days of expansion, surface CAR and intracellular LEF1 expression are determined by flow cytometry. Representative dot plots show LEF1 expression relative to CAR expression from one of two donors in FIG. 35A, panel b.

LEF1 incorporation improves numeric expansion of CAR-NKTs. NKT cell number is determined by trypan blue exclusion assay. Cell count fold-change is calculated between days 2 and 10 post-stimulation for both groups. Mean fold change to the CAR group±SEM (n=8 donors) is shown. **P<0.01, paired Student's t test. The results are presented in FIG. 35A, panel c.

LEF1 incorporation improves CD62L expression of CAR-NKTs. After 10 days of expansion, CD62L expression is assessed by flow cytometry in CAR+-gated NKTs. Representative histograms and mean±SEM of CD62L percentage (n=6 donors) are shown (FIG. 35B, panel d). *P<0.05, paired Student's t test.

LEF1 incorporation reduces TIM-3 expression of CAR-NKTs. After 10 days of expansion, TIM-3 expression is assessed by flow cytometry in CAR+-gated NKTs. Representative histograms and mean fold change to the CAR group±SEM (n=6 donors) is shown (FIG. 35B, panel e). ***P<0.001, paired Student's t test.

LEF1 incorporation reduces short-term cytotoxicity of CAR-NKTs. Luciferase-transduced GD2+ CHLA-255 cells are co-cultured with CAR or CAR-LEF1 NKTs for four hours. Cytotoxicity is calculated from luminescence intensity using non-transduced (NT) NKTs as control. Results from one donor tested is presented in FIG. 35C, panel f.

LEF1 incorporation reduces production of effector cytokines from CAR-NKTs. CAR and CAR-LEF1 NKTs are stimulated with CHLA-255 cells, and supernatants are collected at 24 hours. GM-CSF, IFNγ, TNFα, and IL-4 levels are measured by Luminex assay. Results from one of two donors tested with similar results (FIG. 35C and FIG. 35D, panel g). ***P<0.001, ****P<0.0001, unpaired Student's t test.

### Example 12: CAR-LEF1 NKTs Mediate Superior Tumor Control In Vivo

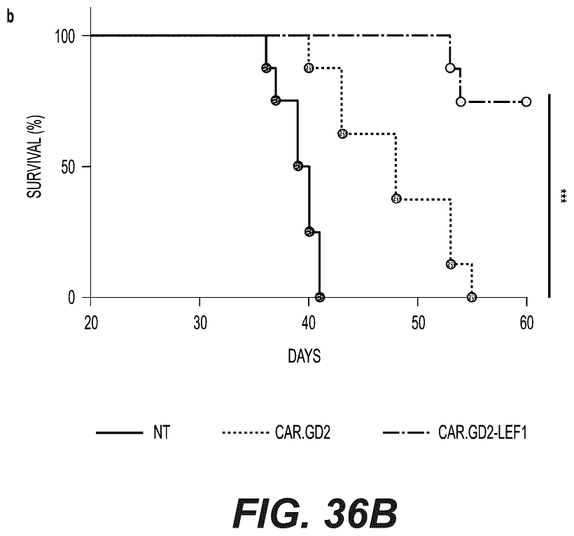

NKTs are expanded with IL-2 and transduced with either CAR.GD2 (CAR) or CAR.GD2-LEF1 (CAR-LEF1), with a non-transduced (NT) control group. NSG mice (n=8 mice per group) are i.v. injected with 1×10⁶ luciferase-transduced CHLA-255 cells on day 0. On day 7, mice receive an i.v. injection of NT, CAR, or CAR-LEF1 NKT preparations (4×10⁶ CAR+ cells per mouse). IL-2 is injected i.p. three times a week for two weeks after NKT injection. Tumor growth is monitored using bioluminescence imaging once per week. The results are shown in FIG. 36A.

The survival of the test mice is generated using the Kaplan-Meier method. Differences in survival probability are compared using the log-rank test. ***P<0.001. The results are presented in FIG. 36B, and in Table 5, below.

Table 6 presents the average and statistical analysis by T-test.

TABLE 5
Liminescence count at week six

| | | | | | | | |
|---|---|---|---|---|---|---|---|
| CAR | 1.20E+05 | 1.98E+05 | 1.90E+05 | 6.07E+05 | 5.97E+05 | 1.28E+05 | 2.24E+05 |
| CAR- | 4.16E+04 | 4.44E+04 | 1.38E+05 | 1.31E+05 | 1.46E+04 | 4.66E+04 | 3.83E+04 |
| LEF1 | | | | | | | |

TABLE 6
Average of Luminescence and T-test

| Average | T-test |
|---|---|
| 2.95E+05 | 0.009867 |
| 5.03E+04 | 6.30E+04 |

1. A genetically engineered natural killer T (NKT) cell comprising an expression construct encoding a protein sequence for a transcriptional activator in the Wnt signaling pathway.

2. The genetically engineered NKT cell of claim 1, wherein said transcriptional activator is selected from the group consisting of lymphoid enhancer binding factor 1 (LEF1, Gene ID 51176), beta-catenin ((CTNNB1, Gene ID 1499)), Smad3 (Gene ID 4088), HNF1 homeobox A (HNF1A, Gene ID: 6927 (alt. TCF1), transcription factor 7 (TCF7, Gene ID:6932 (alt. TCF1) and TLE family member 1, transcriptional corepressor (TLE 1, Gene ID 7088).

3. The genetically engineered NKT cell of claim 2, wherein said LEF1 selected from the group consisting of Reference Sequence (RefSeq) ID NOs: NP_057353.1, NP_001124185.1, and NP_001124186.1.

4. The genetically engineered NKT cell of claim 1, wherein said genetically engineered NKT cell further comprises an expression construct encoding a protein sequence for a chimeric antigen receptor (CAR).

5. The genetically engineered NKT cell of claim 4, wherein said chimeric antigen receptor (CAR) comprises an antigen recognition domain recognizing a cancer antigen.

6. The genetically engineered NKT cell of claim 5, wherein said cancer antigen is selected from the group consisting of CD19, GD2, and GPC3.

7. A population of cells comprising a plurality of genetically engineered NKT cells comprising expression construct encoding a protein sequence for a transcriptional activator in the Wnt signaling pathway.

8. The population of cells of claim 7, wherein said population further comprises cells including Type I NKT cells, Type II NKT cells, irradiated PBMC cells, non-NKT cells, or non-engineered cells.

9. The population of cells of claim 7, wherein said plurality of genetically engineered NKT cells is greater than 10% of the total cell population.

10. The population of cells of claim 7, wherein said plurality of genetically engineered NKT cells comprises at least 50% CD62L(+) NKT cells.

11. The population of cells of claim 7, wherein at least 30 percent of said plurality of genetically engineered NKT cells express LEF1.

12. A therapeutically effective amount of the population of cells of claim 7.

13. A chimeric antigen receptor expression construct comprising (a) a chimeric antigen receptor (CAR) coding sequence comprising an ectodomain sequence, a transmembrane domain sequence, and an endodomain sequence, and (b) a sequence encoding a protein sequence for a transcriptional activator in the Wnt signaling pathway.

14. The chimeric antigen receptor expression construct of claim 13, wherein said transcriptional activator in the Wnt signaling pathway comprises lymphoid enhancer binding factor 1 (LEF1, Gene ID 51176), beta-catenin ((CTNNB1, Gene ID 1499)), Smad3 (Gene ID 4088), HNF1 homeobox A (HNF1A, Gene ID: 6927 (alt. TCF1), transcription factor 7 (TCF7, Gene ID:6932 (alt. TCF1) or TLE family member 1, transcriptional corepressor (TLE 1, Gene ID 7088).

15. The chimeric antigen receptor expression construct of claim 13, wherein said transcriptional activator in the Wnt signaling pathway is LEF1.

16. The chimeric antigen receptor expression construct of claim 13, wherein said antigen recognition domain binds to an antigen selected from CD19, GD2, or GPC3.

17. The chimeric antigen receptor expression construct of claim 13, further comprising an in-frame coding sequence for an additional protein separated by a foot-and-mouth disease virus (FMDV) 2A sequence or a FMDV 2A related cis acting hydrolase element (CHYSEL) sequence.

18. The chimeric antigen receptor expression construct of claim 16, wherein said antigen recognition domain comprises a single-chain variable fragment (scFv) from the CD19-specific antibody FMC-63.

19. The chimeric antigen receptor expression construct of claim 16, wherein antigen recognition domain comprises a single-chain variable fragment (scFv) from the GD2-specific antibody 14G2a.

20. The chimeric antigen receptor expression construct of claim 13, wherein said endodomain comprises the signal sequence of 4-1BB fused in-frame to a CD3-zeta chain.

21. A genetically engineered NKT cell comprising a chimeric antigen receptor expression construct of claim 13.

22. A method of maintaining NKT cell expansion potential comprising the steps of
engineering NKT cells to express at least a protein coding sequence comprising a transcriptional activator in the Wnt signaling pathway; and
culturing said engineered NKT cells to prepare a population of genetically engineered NKT cells with persistent expansion potential.

23. A method of maintaining NKT cell expansion potential of claim 22, wherein said engineering comprises transfecting or transducing said NKT cells with an expression construct comprising (a) a chimeric antigen receptor (CAR) coding sequence comprising an ectodomain sequence, a transmembrane domain sequence, and an endodomain sequence and (b) a sequence encoding a protein sequence for a transcriptional activator in the Wnt signaling pathway.

24. The method of claim 22, wherein said CAR binds to an antigen selected from CD19, GD2, or GPC3.

25. The method of claim 22, wherein said transcriptional activator in the Wnt signaling pathway comprises lymphoid enhancer binding factor 1 (LEFT, Gene ID 51176), beta-catenin ((CTNNB1, Gene ID 1499)), Smad3 (Gene ID 4088), HNF1 homeobox A (HNF1A, Gene ID: 6927 (alt. TCF1), transcription factor 7 (TCF7, Gene ID:6932 (alt. TCF1) or TLE family member 1, transcriptional corepressor (TLE 1, Gene ID 7088).

26. The method of claim 25, wherein said transcriptional activator of the Wnt signaling pathway is LEF1 selected from the group consisting of Reference Sequence (RefSeq) ID NOs: NP_057353.1, NP_001124185.1, and NP_001124186.1.

27. A method of reducing NKT cell exhaustion in an NKT cell population comprising the steps of
engineering NKT cells to express at least a protein coding sequence comprising a transcriptional activator in the Wnt signaling pathway; and
culturing said engineered NKT cells to prepare a population of genetically engineered NKT cells with reduced NKT cell exhaustion.

28. The method of reducing NKT cell exhaustion in an NKT cell population of claim 27, wherein said engineering comprises transfecting or transducing said NKT cells with an expression construct comprising (a) a chimeric antigen receptor (CAR) coding sequence comprising an ectodomain sequence, a transmembrane domain sequence, and an endodomain sequence and (b) a sequence encoding a protein sequence for a transcriptional activator in the Wnt signaling pathway.

29. The method of reducing NKT cell exhaustion in an NKT cell population of claim 27, wherein said transcriptional activator of the Wnt signaling pathway is LEF1 selected from the group consisting of Reference Sequence (RefSeq) ID NOs: NP_057353.1, NP_001124185.1, and NP_001124186.1.
